(19) United States
(12) Patent Application Publication
(10) Pub. No.: US 2014/0249076 A1
(43) Pub. Date: Sep. 4, 2014

(54) AMYLIN-CALCITONIN CHIMERIC PEPTIDES CONJUGATED TO DURATION ENHANCING MOIETIES

(71) Applicants: AMYLIN PHARMACEUTICALS, LLC, SAN DIEGO, CA (US); ASTRAZENECA PHARMACEUTICALS LP, SAN DIEGO, CA (US)

(72) Inventors: Manoj P. Samant, San Diego, CA (US); Ved Srivastava, San Diego, CA (US); Odile Esther Levy, San Diego, CA (US); Christine M. Mack, San Diego, CA (US); William E. Rote, San Diego, CA (US)

(73) Assignees: ASTRAZENECA PHARMACEUTICALS LP; AMYLIN PHARMACEUTICALS, LLC

(21) Appl. No.: 14/352,087

(22) PCT Filed: Oct. 17, 2012
(86) PCT No.: PCT/US2012/060637

§ 371 Date: Apr. 16, 2014

Related U.S. Application Data
(60) Provisional application No. 61/548,404, filed on Oct. 18, 2011.

(51) Int. Cl. A61K 47/48

(52) U.S. Cl. CPC A61K 47/48215
USPC 514/4.8; 530/307; 514/11.9; 514/7.4; 514/6.9

(57) ABSTRACT

Provided herein are amylin-calcitonin peptide conjugates having enhanced duration of biological activity, and methods of use thereof. The amylin-calcitonin peptide conjugates include duration enhancing moieties, such as water soluble polymers and long chain aliphatic groups, bound to the amylin-calcitonin peptide. Methods of use are provided for treatment of an eating disorder, insulin resistance, obesity, overweight, abnormal postprandial hyperglycemia, Type I diabetes, Type II diabetes, gestational diabetes, metabolic syndrome, dumping syndrome, hypertension, dyslipidemia, cardiovascular disease, hyperlipidemia, sleep apnea, cancer, pulmonary hypertension, cholescystitis or osteoarthritis.

### FIELD

The disclosure provides amylin-calcitonin chimeric peptides conjugated to duration enhancing moieties for treating a variety of diseases.

### BACKGROUND

It is believed that certain metabolic pathologies, such as diabetes and obesity, may be associated with psychiatric disorders, such as depression and schizophrenia. Such metabolic pathologies are generally believed to be co-morbid. However, there is now evidence that behavioral and metabolic alterations are physiologically linked in many cases. See e.g., Laugero et al., 2001, Endocrinology 142:2796-2804; Laugero et al., 2002, Endocrinology 143:4552-4562; Dallman et al., 2003, Proc. Natl. Acad. Sci. USA 100:11696-11701; Laugero, 2004, VITAMINS AND HORMONES, Volume 68, Litwack (ed.).

Exemplary of a relationship between metabolic and behavioral functions, it has been found that amylin, amylin agonists and amylin derivatives are useful in treating psychiatric disorders including, but not limited to mood disorders, anxiety disorders, schizophrenia, binge eating, and cognitive impairments. See, e.g., U.S. Published Appl. Nos. 2008/0287355, 2009/0062193, and 2009/0181890. Amylin has a metabolic function in that is a peptide hormone synthesized by pancreatic β-cells that is co-secreted with insulin in response to nutrient intake. The sequence of amylin is highly preserved across mammalian species and has structural similarities to calcitonin gene-related peptide (CGRP), the calcitonins, the intermedins, and adrenomedullin.

Amylin and structurally related peptides can transit the blood brain barrier, thus providing a physiological basis for the psychiatric activity of amylin, amylin analogs, related peptides, and derivatives thereof. For example, it is known that the actions of amylin are mediated, at least in part, by activation of amylin binding sites in the area postrema (AP). Lesioning of this site abolishes the food intake reduction activity of amylin. See Lutz et al., 1998, Peptides 19:309-317; Riediger et al., 2001, Am J Physiol Regul Integr Comp Physiol 281:R1833-R1843; Lutz et al., 2001, Int J Obes Relat Metab Disord 25:1005-1011. Rowland et al., Regul Pept 71:171-174. Endogenous amylin may contribute to the physiological control of food intake as amylin receptor antagonism stimulates feeding in normal, untreated animals. See e.g., Rushing et al., 2001, Endocrinology 142:5035-5038; Reidelberger et al., 2004, Am J Physiol Regul Integr Comp Physiol 287:R568-R574.

It is believed that a common link between metabolic and behavior disease states may be chronic stress and the associated changes in brain corticotropin releasing factor (CRF) and the adrenocortical steroid hormones (GC). Specifically, CRF and GC molecules play critical roles in modulating behavioral, neuroendocrine, autonomic, and metabolic function under both normal and stressful conditions. Chronic stress and the induction of expression and activity of these molecules are highly associated with behavioral diseases like anxiety and depression, and also with some obesities and diabetes. For example, evidence has been put forth that links CRF and adrenocortical abnormalities to the metabolic syndrome, autoimmune inflammatory disorders, acute and chronic neurodegeneration, sleep disorders, chronic pain, eating disorders, chronic anxiety disorder, and major depression. See e.g., Wong et al., 2000, Proc. Natl. Acad. Sci. USA 97:325-330; Sarnyai et al., 2001, Pharmacol. Rev. 53:209-243; Heinrichs et al., 1999, Baillieres Best Pract. Res. Clin. Endocrinol. Metab. 13:541-554; Chrousos, 2000, Int. J. Obes. Relat. Metab. Disord. 24:S50-S55; Peek et al., 1995, Ann. N.Y. Acad. Sci. 771:665-676; Grammatopoulos et al., 1999, Lancet 354:1546-1549; Dallman et al., 2003, Proc. Natl. Acad. Sci. USA 100:11696-11701.

There is a need in the art for new compounds that can treat metabolic and psychiatric conditions with long last effects. The disclosure provides amylin-calcitonin chimeric peptides conjugated with duration enhancing moieties to meet this need.

### SUMMARY

The disclosure provides amylin-calcitonin chimeric peptide conjugates having enhanced duration of biological activity. The peptides included within the peptide conjugates are amylin, calcitonin, and chimera thereof. The peptide conjugates include duration enhancing moieties, such as water soluble polymers and long chain aliphatic groups, bound to the peptides, optionally through linkers.

The disclosure provides amylin-calcitonin peptide conjugates which include a peptide and a duration enhancing moiety covalently linked thereto. The peptide includes an amino acid sequence of residues 1-32 of Formula (I), wherein up to 25% of the amino acids set forth in Formula (I) may be deleted or substituted with a different amino acid:

| | |
|---|---|
| X′-Xaa¹-Cys²-Asn³-Thr⁴-Ala⁵-Thr⁶-Cys⁷-Val⁸-Leu⁹- | (I) |
| Gly10-Arg¹¹-Leu¹²-Ser¹³-Gln¹⁴-Glu¹⁵-Leu¹⁶-His¹⁷- | |
| Arg¹⁸-Leu¹⁹-Gln²⁰-Thr²¹-Tyr²²-Pro²³-Arg²⁴-Thr²⁵- | |
| Asn²⁶-Xaa²⁷-Gly²⁸-Ser²⁹-Asn³⁰-Thr³¹-Xaa³²-X. | |

wherein X′ is hydrogen, an N-terminal capping group, a bond to a duration enhancing moiety, or a linker to a duration enhancing moiety, Xaa¹ is Lys or a bond, Xaa²⁷ is Thr or Val, Xaa³² is Tyr or a bond, and X is substituted or unsubstituted amino, substituted or unsubstituted alkylamino, substituted or unsubstituted dialkylamino, substituted or unsubstituted cycloalkylamino, substituted or unsubstituted arylamino, substituted or unsubstituted aralkylamino, substituted or unsubstituted alkyloxy, substituted or unsubstituted aryloxy, substituted or unsubstituted aralkyloxy, hydroxyl, a bond to a duration enhancing moiety, or a linker to a duration enhancing moiety. The duration enhancing moiety can be covalently linked, optionally through a linker, to a side chain of a linking amino acid residue, X′ or X. The duration enhancing moiety can be covalently linked, optionally through a linker, to a backbone atom of the peptide. In one embodiment, up to 20% of the amino acids set forth in Formula (I) may be deleted or substituted with a different amino acid. In one embodiment, up to 10% of the amino acids set forth in Formula (I) may be deleted or substituted with a different amino acid.

The disclosure provides pharmaceutical compositions which include the amylin-calcitonin peptide conjugates described herein in combination with a pharmaceutically acceptable excipient.

The disclosure provides for the use of the amylin-calcitonin peptide conjugates and pharmaceutical compositions described herein to treat patients having psychiatric diseases (e.g., anxiety disorders, mood disorders, schizophrenia), eating disorders (e.g., anorexia, bulimia, binge-eating disorder), insulin resistance, obesity, overweight, abnormal postprandial hyperglycemia, diabetes (e.g., Type 1, Type 2, gestational), metabolic syndrome, postprandial dumping syndrome, hypertension, dyslipidemia, cardiovascular disease, hyperlipidemia, sleep apnea, cancer, pulmonary hypertension, cholescystitis, and osteoarthritis.

### BRIEF DESCRIPTION OF THE DRAWINGS

FIG. 1A depicts the daily food intake results from the home cage model described herein for mPEG reagents, Cmpd R1, Cmpd R2 and vehicle. FIG. 1B depicts the daily cumulative body weight gain as described herein. Legend: Cmpd R1 (box); Cmpd R2 (triangle); vehicle (filled circle).

FIG. 2 depicts the daily food intake results from the home cage model described herein for Cmpds 2, 152, 153 and 151. Legend: Vehicle (filled circle); Cmpd 2 (open circle); Cmpd 152 (triangle); Cmpd 153 (box); Cmpd 151 (diamond).

FIG. 3 depicts the daily food intake results from the home cage model described herein for Cmpds 151, 160, 161, 162 and 167. Legend: vehicle (filled circle); Cmpd 151 (diamond); Cmpd 160 (triangle tip up); Cmpd 161 (triangle tip down); Cmpd 162 (box); Cmpd 167 (open circle).

FIG. 4 depicts the daily food intake results from the feeding patterns model described herein for Cmpds 151, 154, 155, 157, and R1. Legend: vehicle (filled circle); Cmpd 151 (diamond); Cmpd 154 (triangle tip up); Cmpd 155 (triangle tip down); Cmpd 157 (open circle); Cmpd R1 (box).

FIG. 5 depicts the daily food intake from the feeding patterns model described herein for Cmpds 151, 156, 158 and 159. Legend: vehicle (filled circle); Cmpd 151 (diamond); Cmpd 156 (triangle); Cmpd 158 (open circle); Cmpd 159 (open box).

FIG. 6 depicts the daily food intake from the home cage model described herein for Cmpds 151, 157, 156 and 169. Legend: vehicle (filled circle); Cmpd 151 (diamond); Cmpd 157 (open circle); Cmpd 156 (triangle); Cmpd 169 (box).

FIG. 7 depicts a histogram of the results for the forced swim test described herein for vehicle, Cmpd 1 and Cmpd 151.

FIG. 8 depicts a histogram of the results for the marble burying assay described herein for vehicle (water), Cmpd 1, Cmpd 151, and Cmpd R2. Doses of Cmpds 1, 151 and R2 are indicated in the abscissa of the histogram and were 0.3 mg/kg, 1.0 mg/kg, and 3.0 mg/kg for each test compound.

FIGS. 9A-B depict the results of the stress induced hyperthermia assay described herein for PEG reagents, Cmpd R2 and R1, compared with Cmpd 169. The figures depict histograms of the stress induced hyperthermia (SIH) response as described herein. Doses and pretreatment times are described in the examples below. Legend: FIG. 9A: vehicle (gray histogram block); Cmpd 169 (checked histogram block); Cmpd R2 (horizontal striped histogram block); FIG. 9B: vehicle (gray histogram block); Cmpd 169 (checked histogram block); Cmpd R1 (horizontal striped histogram block).

FIGS. 10A-D depict the results of the SIH assay described herein comparing Cmpd 169 with Cmpd 1. The figures depict histograms of the stress induced hyperthermia (SIH) response as described herein. Doses and pretreatment times are described in the examples below. Legend: FIG. 10A: vehicle (gray histogram block); Cmpd 1 (checked histogram block); Cmpd 169 (horizontal striped histogram block); FIG. 10B: vehicle (gray histogram block); Cmpd 1 (checked histogram block); Cmpd 169 (horizontal striped histogram block); FIG. 10C: vehicle (gray histogram block); Cmpd 169 (checked histogram block); Cmpd 1 (horizontal striped histogram block); FIG. 10D: vehicle (gray histogram block); Cmpd 169 (checked histogram block); Cmpd 1 (horizontal striped histogram block).

FIG. 11 depicts the results of the SIH assay described herein comparing Cmpd 169 and Cmpd 195. The figure depicts a histogram of the stress induced hyperthermia (SIH) response as described herein. Dose and pretreatment time are described in the example below. Legend: vehicle (gray histogram block); Cmpd 185 (checked histogram block); Cmpd 169 (horizontal striped histogram block).

FIG. 12 depicts the results of the SIH assay described herein comparing Cmpd 176 and Cmpd 1. The figure depicts a histogram of the stress induced hyperthermia (SIH) response as described herein. Dose and pretreatment time are described in the examples below. Legend: vehicle (gray histogram block); Cmpd 176 (checked histogram block); Cmpd 1 (horizontal striped histogram block).

FIG. 13 depicts the results of the SIH assay described herein comparing Cmpd 157 and Cmpd 1. The figure depicts a histogram of the stress induced hyperthermia (SIH) response as described herein. Dose and pretreatment time are described in the examples below. Legend: vehicle (gray histogram block); Cmpd 157 (checked histogram block); Cmpd 1 (horizontal striped histogram block).

FIG. 14 depicts the results of the SIH assay described herein comparing Cmpd 170 and Cmpd 1. The figure depicts a histogram of the stress induced hyperthermia (SIH) response as described herein. Dose and pretreatment time are described in the examples below. Legend: vehicle (gray histogram block); Cmpd 170 (checked histogram block); Cmpd 1 (horizontal striped histogram block).

FIGS. 15A-C depict the results of the SIH assay described herein comparing Cmpd 156 and Cmpd 1. The figures depict histograms of the stress induced hyperthermia (SIH) response as described herein. Doses and pretreatment times are described in the examples below. Legend: FIG. 15A: vehicle (gray histogram block); Cmpd 156 (checked histogram block); Cmpd 1 (horizontal striped histogram block); FIG. 15B: vehicle (gray histogram block); Cmpd 1 (checked histogram block); Cmpd 156 (horizontal striped histogram block); FIG. 15C: vehicle (gray histogram block); Cmpd 1 (checked histogram block); Cmpd 156 (horizontal striped histogram block).

FIGS. 16A-B depict the results of the SIH assay described herein comparing Cmpd 171 and Cmpd 1. The figures depict a histogram of the stress induced hyperthermia (SIH) response as described herein. Doses and pretreatment times are described in the examples below. Legend: FIG. 16A: vehicle (gray histogram block); Cmpd 171 (checked histogram block); Cmpd 1 (horizontal striped histogram block); FIG. 16B: vehicle (gray histogram block); Cmpd 171 (checked histogram block); Cmpd 1 (horizontal striped histogram block).

FIGS. 17A-C depict the results of the SIH assay described herein comparing Cmpd 151 and Cmpd 1. The figures depict histograms of the stress induced hyperthermia (SIH) response as described herein. Doses and pretreatment times are described in the examples below. Legend:

FIG. 17A: vehicle (box or gray histogram block); Cmpd 1 (checked histogram block); Cmpd 151 (horizontal striped histogram block); FIG. 17B: vehicle (gray histogram block); Cmpd 1 (checked histogram block); Cmpd 151 (horizontal striped histogram block); FIG. 17C: vehicle (gray histogram block); Cmpd 1 (checked histogram block); Cmpd 151 (horizontal striped histogram block).

FIG. 18 depicts the results of the SIH assay described herein comparing Cmpd 152 and Cmpd 1. The figure depicts a histogram of the stress induced hyperthermia (SIH) response as described herein. Dose and pretreatment time are described in the examples below. Legend: vehicle (gray histogram block); Cmpd 152 (checked histogram block); Cmpd 1 (horizontal striped histogram block).

FIG. 19 depicts the results of a cumulative mouse food intake assay described herein. Concentration conditions are provided in the figure. Legend: vehicle (filled box); Cmpd 18 (filled triangle); Cmpd 1 (open box); Cmpd 189 (cross); Cmpd 187 (circle); Cmpd 193 (open triangle).

FIG. 20 depicts the results of the SIH assay described herein comparing Cmpd 189 and Cmpd 1. The figure depicts a histogram of the stress induced hyperthermia (SIH) response as described herein. Dose and pretreatment time are described in the examples below. Legend: vehicle (gray histogram block); Cmpd 189 (checked histogram block); Cmpd 1 (horizontal striped histogram block).

FIGS. 21A-B depict the time course for mean (+/−standard deviation SD) plasma concentration of Cmpd 151 as described herein. Legend: FIG. 21A: linear concentration ordinate in ng/mL of Cmpd 151; IV administration (filled circle); SC administration (open circle); FIG. 21B: the data of FIG. 21A plotted as a semi-logarithmic plot.

FIG. 22 depicts the results of the SIH assay described herein comparing Cmpd 169 and Cmpd 171. The figure depicts a histogram of the stress induced hyperthermia (SIH) response as described herein. Dose and pretreatment time are described in the examples below. Legend: vehicle (gray histogram block); Cmpd 169 (checked histogram block); Cmpd 171 (horizontal striped histogram block).

FIG. 23 depicts the results of the SIH assay described herein comparing Cmpds 171, 183 and 181. The figure depicts a histogram of the stress induced hyperthermia (SIH) response as described herein. Dose and pretreatment time are described in the examples below. Legend: vehicle (gray histogram block); Cmpd 171 (checked histogram block); Cmpd 183 (horizontal striped histogram block); Cmpd 181 (vertically striped histogram block).

FIG. 24 depicts the results of the SIH assay described herein comparing Cmpds 169, 180 and 179. The figure depicts a histogram of the stress induced hyperthermia (SIH) response as described herein. Dose and pretreatment time are described in the examples below. Legend: vehicle (gray histogram block); Cmpd 169 (checked histogram block); Cmpd 180 (horizontal striped histogram block); Cmpd 179 (vertically striped histogram block).

FIG. 25 depicts the results of the SIH assay described herein comparing Cmpd 169 and Cmpd 182. The figure depicts a histogram of the stress induced hyperthermia (SIH) response as described herein. Dose and pretreatment time are described in the examples below. Legend: vehicle (gray histogram block); Cmpd 169 (checked histogram block); Cmpd 182 (horizontal striped histogram block).

### DETAILED DESCRIPTION

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo,” as used herein, means an oxygen that is double bonded to a carbon atom.

The term “alkylsulfonyl,” as used herein, means a moiety having the formula —S(O₂)—R′, where R′ is an alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, and —NO₂ in a number ranging from zero to (2 m′+1), where m′ is the total number of carbon atoms in such radical. R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a peptide conjugate of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR′″, —NR—C(NR′R″)═NR′″, —S(O)R′, —S(O)₂R′, —S(O)₂NR′R″, —NRSO₂R′, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a peptide conjugate of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR^{†}—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties: (A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and (B) alkyl, heteroalkyl, cycloalkyl, heterocycloalkyl, aryl, and heteroaryl, substituted with at least one substituent selected from: (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (ii) alkyl, heteroalkyl, cycloalkyl, heterocycloalkyl, aryl, and heteroaryl, substituted with at least one substituent selected from: (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (b) alkyl, heteroalkyl, cycloalkyl, heterocycloalkyl, aryl, or heteroaryl, substituted with at least one substituent selected from: oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

The term “pharmaceutically acceptable salts” is meant to include salts of the active peptide conjugates that are prepared with relatively nontoxic acids or bases, depending on the particular substituents found on the peptide conjugates described herein. When peptide conjugates contain relatively acidic functionalities, base addition salts can be obtained by contacting the neutral form of such peptide conjugates with a sufficient amount of the desired base, either neat or in a suitable inert solvent. Examples of pharmaceutically acceptable base addition salts include sodium, potassium, calcium, ammonium, organic amino, or magnesium salt, or a similar salt. When peptide conjugates described herein contain relatively basic functionalities, acid addition salts can be obtained by contacting the neutral form of such peptide conjugates with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., 1977, “Pharmaceutical Salts”, Journal of Pharmaceutical Science 66:1-19.

Thus, the peptide conjugates described herein may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the peptide conjugates are preferably regenerated by contacting the salt with a base or acid and isolating the parent peptide in the conventional manner. The parent form of the peptide conjugates differs from the various salt forms in certain physical properties, such as solubility in polar solvents.

The peptide conjugates described herein may also contain unnatural proportions of atomic isotopes at one or more of the atoms that constitute such peptides. For example, the peptides may be radiolabeled with radioactive isotopes, such as for example tritium (³H), iodine-125 (¹²⁵I), or carbon-14 (¹⁴C). All isotopic variations of the peptides of the present invention, whether radioactive or not, are encompassed within the scope of the present invention.

The symbol “ [symbol] ” denotes the point of attachment of a chemical moiety to the remainder of a molecule or chemical formula.

“Analog” as used herein in the context of peptides refers to a peptide that has insertions, deletions and/or substitutions of amino acids relative to a parent peptide. An analog may have superior stability, solubility, efficacy, half-life, and the like. In some embodiments, an analog is a peptide having at least 50%, for example 50%, 55%, 60%, 65%, 70%, 75%, 80%, 85%, 90%, 95%, 98%, or even higher, sequence identity to the parent peptide. In one embodiment, the parent peptide described herein is davalintide and the analog is a davalintide analog that has at least 50% sequence identity to davalintide; or at least 60% sequence identity to davalintide; or at least 75% sequence identity to davalintide; or at least 80% sequence identity to davalintide; or at least 85% sequence identity to davalintide; or at least 90% sequence identity to davalintide; or at least 92% sequence identity to davalintide; or at least 95% sequence identity to davalintide; or at least 98% sequence identity to davalintide. In one embodiment, the parent peptide described herein is Cmpd 2 and the analog is a Cmpd 2 analog that has at least 50% sequence identity to Cmpd 2; or at least 60% sequence identity to Cmpd 2; or at least 75% sequence identity to Cmpd 2; or at least 80% sequence identity to Cmpd 2; or at least 85% sequence identity to Cmpd 2; or at least 90% sequence identity to Cmpd 2; or at least 92% sequence identity to Cmpd 2; or at least 95% sequence identity to Cmpd 2; or at least 98% sequence identity to Cmpd 2.

The terms “identity,” “sequence identity” and the like in the context of comparing two or more nucleic acids or peptide sequences, refer to two or more sequences or subsequences that are the same or have a specified percentage of amino acid residues or nucleotides that are the same (i.e., about 50% identity, preferably 50%, 55%, 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a sequence comparison algorithms as known in the art, for example BLAST or BLAST 2.0. This definition includes sequences that have deletions and/or additions, as well as those that have substitutions, as well as naturally occurring, e.g., polymorphic or allelic variants, and man-made variants. In preferred algorithms, account is made for gaps and the like, as known in the art. For sequence comparison, typically one sequence acts as a reference sequence, to which test sequences are compared. When using a sequence comparison algorithm, test and reference sequences are entered into a computer, subsequence coordinates are designated if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters. Optimal alignment of sequences for comparison can be conducted, e.g., by the local homology algorithm of Smith & Waterman, 1981, Adv. Appl. Math. 2:482, by the homology alignment algorithm of Needleman & Wunsch, 1970, J. Mol. Biol. 48:443, by the search for similarity method of Pearson & Lipman, 1988, Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection. See, e.g., Current Protocols in Molecular Biology, Ausubel et al., eds., 1995 supplement. Preferred examples of algorithms that are suitable for determining percent sequence identity and sequence similarity include the BLAST and BLAST 2.0 algorithms, which are described in Altschul et al., 1977, Nucl. Acids Res. 25:3389-3402 and Altschul et al., 1990, J. Mol. Biol. 215:403-410. BLAST and BLAST 2.0 are used, as known in the art, to determine percent sequence identity for the nucleic acids and proteins of the invention. Software for performing BLAST analyses is publicly available through the web site of the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., id.). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, 1989, Proc. Natl. Acad. Sci. USA 89:10915) alignments (B) of 50, expectation (E) of 10, M=5, N=−4, and a comparison of both strands.

To determine the percent identity or similarity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same or similar amino acid residue or nucleotide as the corresponding position in the second sequence, then the molecules are identical or similar at that position. The percent identity or similarity between the two sequences is a function of the number of identical or similar positions shared by the sequences (i.e., % identity=# of identical positions/total # of positions (e.g., overlapping positions)×100). The similarity of two amino acids can be assessed by a variety of methods known in the art. For example, nonpolar neutral residues (e.g., Ala, Cys, Gly, Ile, Leu, Met, Phe, Pro, Trp, Val) can be considered similar, as can in turn acidic charged polar (e.g., Glu, Asp), basic charged polar (e.g., Arg, H is, Lys) and neutral polar (e.g., Asn, Gln, Ser, Thr, Tyr) residues.

Both identity and similarity may be readily calculated. For example, in calculating percent identity, only exact matches may be counted, and global alignments may be performed as opposed to local alignments. Methods commonly employed to determine identity or similarity between sequences include, e.g., those disclosed in Carillo et al., 1988, SIAM J. Applied Math. 48:1073. Exemplary methods to determine identity are designed to give the largest match between the sequences tested. Exemplary methods to determine identity and similarity are also provided in commercial computer programs. A particular example of a mathematical algorithm utilized for the comparison of two sequences is the algorithm of Karlin et al., 1990, Proc. Natl. Acad. Sci. USA 87:2264-2268, and as modified e.g., as in Karlin et al., 1993, Proc. Natl. Acad. Sci. USA 90:5873-5877. Such an algorithm is incorporated into the NBLAST and XBLAST programs of Altschul et al., 1990, J. Mol. Biol. 215:403-410. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search, which detects distant relationships between molecules. When utilizing BLAST, Gapped BLAST, and PSI-Blast programs, the default parameters of the respective programs (e.g., XBLAST and NBLAST) can be used, as known in the art. Additionally, the FASTA method (Atschul et al., 1990, id.) can be used. Another particular example of a mathematical algorithm useful for the comparison of sequences is the algorithm of Myers et al., 1988, CABIOS 4:11-17. Such an algorithm is incorporated into the ALIGN program (version 2.0), which is part of the GCG sequence alignment software package (Devereux et al., 1984, Nucleic Acids Res. 12(1):387). Percent identity can be determined by analysis with the AlignX® module in Vector NTI® (Invitrogen; Carlsbad Calif.).

“Patient” refers to mammals, i.e., warm-blooded animals. Patients include humans; companion animals (e.g., dogs, cats); farm animals; wild animals; and the like. In one embodiment, the patient is a human. In one embodiment, the patient is a cat or dog.

“Amylin agonist compounds” include native amylin peptides, amylin analog peptides, and other compounds (e.g., small molecules) that have amylin agonist activity. “Amylin agonist compounds” include amylin-calcitonin chimeric peptides. The “amylin agonist compounds” can be derived from natural sources, can be synthetic, or can be derived from recombinant DNA techniques. Amylin agonist compounds have amylin agonist receptor binding activity and may comprise amino acids (e.g., natural, unnatural, or a combination thereof), peptide mimetics, chemical moieties, and the like. The skilled artisan will recognize amylin agonist compounds using amylin receptor binding assays or by measuring amylin agonist activity in soleus muscle assays. Amylin agonist compounds can have an IC₅₀ of about 200 nM or less, about 100 nM or less, or about 50 nM or less, in an amylin receptor binding assay, such as that described herein, in U.S. Pat. No. 5,686,411, and US Publication No. 2008/0176804, the disclosures of which are incorporated by reference herein. The term “IC₅₀” refers to the half maximal inhibitory concentration of a compound inhibiting a biological or biochemical function. Accordingly, in the context of receptor binding studies, IC₅₀ refers to the concentration of a test compound which competes half of a known ligand from a specified receptor. Amylin agonist compounds can have an EC₅₀ of about 20 nM or less, about nM 15 or less, about nM 10 or less, or about nM 5 or less in a soleus muscle assay, such as that described herein and in U.S. Pat. No. 5,686,411. The term “EC₅₀” refers to the effective concentration of a compound which induces a response halfway between a baseline response and maximum response, as known in the art. Amylin agonist compound can have at least 90% or 100% sequence identity to [25,28,29Pro]human-amylin (pramlintide). The amylin agonist compound can be a peptide chimera of amylin (e.g., human amylin, rat amylin, and the like) and calcitonin (e.g., human calcitonin, salmon calcitonin, and the like). Suitable and exemplary amylin agonist compounds are described herein and are also described in US Publication No. 2008/0274952, the disclosure of which is incorporated by reference herein. Unless indicated differently, the term “about” in the context of a numeric value refers to +/−10% of the numeric value.

The term “parent” in the context of peptides refers to a peptide which serves as a reference structure prior to modification, e.g., insertion, deletion and/or substitution. The terms “conjugate” and “peptide conjugate” and the like in the context of compounds useful in the methods described herein refer to peptides which are bound to one or more duration enhancing moieties, optionally through a linker.

The term “peptide” refers to a polymer of amino acids connected by amide bonds. The terms “des-amino acid,” “des-AA,” “desLys” and the like refer to the absence of the indicated amino acid. An amino acid (or functionality) being “absent” means that the residue (or functionality) formerly attached at the N-terminal and C-terminal side of the absent amino acid (or functionality) have become bonded together.

“Derivative” in the context of a peptide refers to a molecule having the amino acid sequence of a parent or analog thereof, but additionally having a chemical modification of one or more of its amino acid side groups, α-carbon atoms, backbone nitrogen atoms, terminal amino group, or terminal carboxylic acid group. A chemical modification includes, but is not limited to, adding chemical moieties, creating new bonds, and removing chemical moieties. Modifications at amino acid side groups include, but are not limited to, acylation of lysine ε-amino groups, N-alkylation of arginine, histidine, or lysine, alkylation of glutamic or aspartic carboxylic acid groups, and deamidation of glutamine or asparagine. Modifications of the terminal amino include, but are not limited to, the desamino, N-lower alkyl, N-di-lower alkyl, constrained alkyls (e.g. branched, cyclic, fused, adamantyl) and N-acyl modifications. Modifications of the terminal carboxy group include, but are not limited to, the amide, lower alkyl amide, constrained alkyls (e.g. branched, cyclic, fused, adamantyl) alkyl, dialkyl amide, and lower alkyl ester modifications. Furthermore, one or more side groups, or terminal groups, may be protected by protective groups known to the ordinarily-skilled synthetic chemist. The alpha-carbon of an amino acid may be mono- or dimethylated. Derivatives of the peptides described herein are also contemplated wherein the stereochemistry of individual amino acids may be inverted from (L)/S to (D)/R at one or more specific sites. Also contemplated are peptides modified by glycosylation, at e.g., Asn, Ser and/or Thr residues.

Throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

The disclosure provides a peptide conjugate which includes a peptide to which one or more duration enhancing moieties are linked, optionally through a linker. Linkage of the duration enhancing moiety to the peptide can be through a linker as described herein. Alternatively, linkage of the duration enhancing moiety to the peptide can be via a direct covalent bond. The duration enhancing moiety can be a water soluble polymer, or a long chain aliphatic group, as described herein. In some embodiments, a plurality of duration enhancing moieties are attached to the peptide, in which case each linker to each duration enhancing moiety is independently selected from the linkers described herein.

In some embodiments, amylin-calcitonin peptide conjugates include an amino acid sequence of residues 1-32 of Formula (I) following, wherein up to 25% of the amino acids set forth in Formula (I) may be deleted or substituted with a different amino acid:

| | |
|---|---|
| X′-Xaa¹-Cys²-Asn³-Thr⁴-Ala⁵-Thr⁶-Cys⁷-Val⁸-Leu⁹- | (I) |
| Gly¹⁰-Arg¹¹-Leu¹²-Ser¹³-Gln¹⁴-Glu¹⁵-Leu¹⁶-His¹⁷-Arg¹⁸- | |
| Leu¹⁹-Gln²⁰-Thr²¹-Tyr²²-Pro²³-Arg²⁴-Thr²⁵-Asn²⁶-Xaa²⁷- | |
| Gly²⁸-Ser²⁹-Asn³⁰-Thr³¹-Xaa³²-X. | |

In Formula (I), X′ is hydrogen, an N-terminal capping group, a bond to a duration enhancing moiety, or a linker to a duration enhancing moiety. Xaa¹ is Lys or a bond, Xaa²⁷ is Thr or Val, and Xaa³² is Tyr or a bond. In one embodiment, up to 20% of the amino acids set forth in Formula (I) may be deleted or substituted with a different amino acid. In one embodiment, up to 10% of the amino acids set forth in Formula (I) may be deleted or substituted with a different amino acid. A person having ordinary skill in the art will immediately recognize that the peptide of Formula (I), and other formulae disclosed herein, has an appropriate valency in order to attach to one or more duration enhancing moieties. For example, where a single duration enhancing moiety is present, the peptide of Formula (I) is a monovalent peptide, which valency attaches to the duration enhancing moiety, optionally through a linker. Accordingly, where two duration enhancing moietites are present, the peptide of Formula (I) is a divalent peptide, and so forth.

Further regarding Formula (I), the variable X represents a C-terminal functionality (e.g., a C-terminal cap). X is substituted or unsubstituted amino, substituted or unsubstituted alkylamino, substituted or unsubstituted dialkylamino, substituted or unsubstituted cycloalkylamino, substituted or unsubstituted arylamino, substituted or unsubstituted aralkylamino, substituted or unsubstituted alkyloxy, substituted or unsubstituted aryloxy, substituted or unsubstituted aralkyloxy, hydroxyl, a bond to a duration enhancing moiety, or a linker to a duration enhancing moiety. In some embodiments, the duration enhancing moiety is covalently linked, optionally through a linker, to a side chain of a linking amino acid residue, X′ or X. In some embodiments, the duration enhancing moiety is covalently linked, optionally through a linker, to a backbone atom of the peptide. If the C-terminal of the peptide with the sequence of residues 1-32 of any of Formulae (I)-(II) is capped with a functionality X, then X is preferably amine thereby forming a C-terminal amide. The N-terminal of peptides described herein, including the peptides according to Formulae (I)-(II), can be covalently linked to a variety of functionalities including, but not limited to, the acetyl group. The term “N-terminal capping group” refers to a moiety covalently bonded to the N-terminal nitrogen of a peptide, e.g., substituted or unsubstituted acyl, substituted or unsubstituted acyloxy, Schiff's bases, and the like, as known in the art. In some embodiments, the N-terminal functionality X′ is an amine-protecting group as known in the art, preferably Fmoc.

In some embodiments, up to 5%, 10%, 15%, 20%, 25%, 30%, 35%, 40%, 45% or even 50% of the amino acids of residues 1-32 of Formula (I) are deleted or substituted in a peptide according to Formula (I). In some embodiments, the peptide has 0, 1, 2, 3, 4, 5, 6, 7, 8, 9, 10, 11, 12, 13, 14, 15 or even 16 amino acid substitutions relative to the amino acid sequence set forth in Formula (I).

In some embodiments, the peptide of the peptide conjugate has a sequence which has a defined sequence identity with respect to the residues 1-32 of the amino acid sequence according to Formula (I).

In some embodiments, the sequence identity between a peptide described herein and residues 1-32 Formula (I) is 50%, 55%, 60%, 65%, 70%, 75%, 80%, 85%, 90%, 95% or even higher. In some embodiments, up to 50%, 45%, 40%, 35%, 30%, 25%, 20%, 15%, 10%, 5% or even less of the amino acids set forth in residues 1-32 of Formulae (I)-(II) may be deleted or substituted with a different amino acid. In some embodiments, the sequence identity is within the range 75%-100%. In some embodiments, the sequence identity is within the range 75%-90%. In some embodiments, the sequence identity is within the range 80%-90%. In some embodiments, the sequence identity is at least 75%. In some embodiments, the peptide of the conjugate has the sequence of residues 1-32 of Formula (I).

In some embodiments, the peptide has the sequence of Cmpd 1. In some embodiments, the peptide has the sequence of Cmpd 18. In some embodiments, the peptide has one or more conservative amino acid substitutions with respect to the sequence of Formula (I). “Conservative amino acid substitution” refers to substitution of amino acids having similar biochemical properties at the side chain (e.g., hydrophilicity, hydrophobocity, charge type, van der Waals radius, and the like). “Non-conservative amino acid substitution” refers to substitution of amino acids having dissimilar biochemical properties at the side chain.

It is understood that in the calculation of sequence identity with respect to any of the peptides set forth herein (e.g., as found in residues 1-32 of Formulae (I)-(II), the sequence to be compared is taken over the amino acids disclosed therein, irrespective of any N-terminal (i.e., X′) or C-terminal (i.e., X) functionality present. It is further understood that the presence of a duration enhancing moiety covalently linked to the side chain of an amino acid is immaterial to the calculation of sequence identity. For example, a lysine substituted at any position of Formulae (I)-(II) and additionally bonded, optionally through a linker, with a duration enhancing moiety is a lysine for purposes of sequence identity calculation.

In another aspect, there is provided a peptide which includes an amino acid sequence of residues 1-32 of Formula (II) following, wherein up to 25% of the amino acids set forth in Formula (II) may be deleted or substituted with a different amino acid:

| | |
|---|---|
| X′-Xaa¹-Xaa²-Asn³-Thr⁴-Ala⁵-Thr⁶-Xaa⁷-Val⁸-Leu⁹- | (II) |
| Gly¹⁰-Arg¹¹-Leu¹²-Ser¹³-Gln¹⁴-Glu¹⁵-Leu¹⁶-His¹⁷- | |
| Arg¹⁸-Leu¹⁹-Gln²⁰-Thr²¹-Tyr²²-Pro²³-Arg²⁴-Thr²⁵- | |
| Asn²⁶-Xaa²⁷-Gly²⁸-Ser²⁹-Asn³⁰-Thr³¹-Xaa³²-X | |

Regarding Formula (II), in some embodiments, Xaa¹ is a bond, Lys, or a thiol containing residue capable of forming an intramolecular disulfide bond with the side chain of residue Xaa⁷, Xaa² is any amino acid or a thiol containing moiety capable of forming an intramolecular disulfide bond with the side chain of residue Xaa⁷, Xaa⁷ is Cys or a thiol containing residue capable of forming an intramolecular disulfide bond with the side chain of either of residue Xaa¹ or Xaa², Xaa²⁷ is Thr or Val, and Xaa³² is Tyr or a bond, provided that if Xaa¹ is a thiol containing residue capable of forming an intramolecular disulfide bond with the side chain of residue Xaa⁷, then Xaa² is not a thiol containing residue capable of forming an intramolecular disulfide bond with the side chain of residue Xaa⁷. Exemplary thiol containing moieties suitable for Xaa¹ or Xaa² include, but are not limited to, 3-mercaptopropionic acid and higher order homologs thereof (e.g., C₄-C₆), acetyl penicillamine, desamino penicillamine, acetyl-alpha-methyl cysteine, 2-methyl-3-mercaptopropionic acid, acetyl-norcysteine, and the like. X′ and X in Formula (II) are as defined for Formula (I).

In some embodiments, Xaa¹ is Cys, or a thiol containing residue capable of forming an intramolecular disulfide bond with the side chain of residue Xaa⁷, Xaa² is any amino acid, Xaa⁷ is Cys or a thiol containing residue capable of forming an intramolecular disulfide bond with the side chain of residue Xaa¹, Xaa²⁷ is Thr or Val, and Xaa³² is Tyr or a bond. Exemplary thiol containing moieties suitable for Xaa¹ include, but are not limited to, the moieties set forth above. In certain embodiments, Xaa² is not Cys.

In some embodiments, Xaa¹ is a moiety capable of forming an intramolecular lanthionine type bond with the side chain of residue Xaa⁷, Xaa² is any amino acid, Xaa⁷ is a moiety capable of forming an intramolecular lanthionine type bond with the side chain of residue Xaa¹, Xaa²⁷ is Thr or Val, and Xaa³² is Tyr or a bond. Preferably, Xaa² is not Cys. X′ and X in Formula (II) are as defined for Formula (I).

In some embodiments, the peptide has 0, 1, 2, 3, 4, 5, 6, 7, 8, 9, 10, 11, 12, 13, 14, 15 or even 16 amino acid deletions or substitutions relative to the amino acid sequence set forth in Formula (II).

In some embodiments, up to 20% of the amino acids set forth in Formula (II) may be deleted or substituted with a different amino acid. In some embodiments, up to 15% of the amino acids set forth in Formula (II) may be deleted or substituted with a different amino acid. In some embodiments, up to 10% of the amino acids set forth in Formula (II) may be deleted or substituted with a different amino acid. In some embodiments, up to 5% of the amino acids set forth in Formula (II) may be deleted or substituted with a different amino acid.

In some embodiments, there is provided a peptide conjugate which includes a peptide to which one or more duration enhancing moieties are linked, optionally through a linker. The peptide of the peptide conjugate includes a sequence having a defined sequence identity with respect to the amino acid sequence of residues 1-32 according to Formula (II). In some embodiments, the sequence identity between a compound described herein and residues 1-32 of Formula (II) is 50%, 55%, 60%, 65%, 70%, 75%, 80%, 85%, 90%, or 95%. In some embodiments, the sequence identity is at least 75%. In some embodiments, the sequence identity is at least 80%. In some embodiments, the sequence identity is at least 90%.

Peptides including the sequence of residues 1-32 of Formulae (I)-(II) can be considered to be chimeric combinations of amylin and calcitonin, or analogs thereof. Amylin is a peptide hormone synthesized by pancreatic β-cells that is co-secreted with insulin in response to nutrient intake. The sequence of amylin is highly preserved across mammalian species, with structural similarities to calcitonin gene-related peptide (CGRP), the calcitonins, the intermedins, and adrenomedullin. The glucoregulatory actions of amylin complement those of insulin by regulating the rate of glucose appearance in the circulation via suppression of nutrient-stimulated glucagon secretion and slowing gastric emptying. In insulin-treated patients with diabetes, pramlintide, a synthetic and equipotent analogue of human amylin, reduces postprandial glucose excursions by suppressing inappropriately elevated postprandial glucagon secretion and slowing gastric emptying. The sequences of rat amylin, human amylin and pramlintide follow, respectively:

(SEQ ID NO: 1)
KCNTATCATQRLANFLVRSSNNLGPVLPPTNVGSNTY;
(SEQ ID NO: 2)
KCNTATCATQRLANFLVHSSNNFGAILSSTNVGSNTY;
(SEQ ID NO: 3)
KCNTATCATQRLANFLVHSSNNFGPILPPTNVGSNTY.

Davalintide (Cmpd 18) is a potent amylin agonist useful in the treatment of a variety of disease indications. See WO 2006/083254 and WO 2007/114838, each of which is incorporated by reference herein in its entirety and for all purposes. Davalintide is a chimeric peptide, having an N-terminal loop region of amylin or calcitonin and analogs thereof, an alpha-helical region of at least a portion of an alpha-helical region of calcitonin or analogs thereof or an alpha-helical region having a portion of an amylin alpha-helical region and a calcitonin alpha-helical region or analog thereof, and a C-terminal tail region of amylin or calcitonin. The sequences of human calcitonin, salmon calcitonin and davalintide follow, respectively:

(SEQ ID NO: 4)
CGNLSTCMLGTYTQDFNKFHTFPQTAIGVGAP;
(SEQ ID NO: 5)
CSNLSTCVLGKLSQELHKLQTYPRTNTGSGTP;
(SEQ ID NO: 6)
KCNTATCVLGRLSQELHRLQTYPRTNTGSNTY.

The terms “linker” and the like, in the context of attachment of duration enhancing moieties to a peptide in a peptide conjugate described herein, means a divalent species (-L-) covalently bonded in turn to a peptide having a valency available for bonding and to a duration enhancing moiety having a valency available for bonding. The available bonding site on the peptide is conveniently a side chain residue (e.g., lysine, cysteine, aspartic acid, and homologs thereof). In some embodiments, the available bonding site on the peptide is the side chain of a lysine or a cysteine residue. In some embodiments, the available bonding site on the peptide is the N-terminal amine. In some embodiments, the available bonding site on the peptide is the C-terminal carboxyl. In some embodiments, the available bonding site on the peptide is a backbone atom thereof. As used herein, the term “linking amino acid residue” means an amino acid within residues 1-32 of Formulae (I)-(II) to which a duration enhancing moiety is attached, optionally through a linker.

In some embodiments, compounds are provided having a linker covalently linking a peptide with a duration enhancing moiety. The linker is optional; i.e., any linker may simply be a bond. In some embodiments, the linker is attached at a side chain of the peptide. In some embodiments, the linker is attached to a backbone atom of the peptide.

In one embodiment, the linker is a polyfunctional amino acid, for example but not limited to, lysine and homologs thereof, aspartic acid and homologs thereof, and the like. The term “polyfunctional” in the context of an amino acid refers to a side chain functionality which can react to form a bond, in addition to the alpha amine and carboxyl functionalities of the amino acid. Exemplary functionalities of polyfunctional amino acids include, but are not limited to, amine, carboxyl and sulfhydryl functionalities.

In some embodiments, the linker comprises from 1 to 30 amino acids (“peptide linker”) linked by peptide bonds. The amino acids can be selected from the 20 naturally occurring amino acids. Alternatively, non-natural amino acids can be incorporated either by chemical synthesis, post-translational chemical modification or by in vivo incorporation by recombinant expression in a host cell. Some of these linker amino acids may be glycosylated. In another embodiment the 1 to 30 amino acids are selected from glycine, alanine, proline, asparagine, glutamine, and lysine. In some embodiments, the linker is made up of a majority of amino acids that are sterically unhindered, such as glycine, alanine and/or serine. Polyglycines are particularly useful, e.g. (Gly)₃, (Gly)₄, (Gly)₅, as are polyalanines, poly(Gly-Ala) and poly(Gly-Ser). Other specific examples of linkers are (Gly)₃Lys(Gly)₄; (Gly)₃AsnGlySer(Gly)₂; (Gly)₃Cys(Gly)₄; and GlyProAsnGlyGly. Combinations of Gly and Ala are particularly useful as are combination of Gly and Ser. Thus in a further embodiment the peptide linker is selected from the group consisting of a glycine rich peptide, e.g. Gly-Gly-Gly; the sequences [Gly-Ser]ₙ, [Gly-Gly-Ser]ₙ, [Gly-Gly-Gly-Ser]ₙ and [Gly-Gly-Gly-Gly-Ser]ₙ, where n is 1, 2, 3, 4, 5 or 6, for example [Gly-Gly-Gly-Gly Ser]₃.

In some embodiments, the linker includes a divalent heteroatom. In some embodiments, the linker is, or includes, —O—, —S—, —S—S—, —OCO—, —OCONH—, and —NHCONH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. Representative linkers include —O—, —S—, —S—S—, —OCO—, —OCONH—, and —NHCONH—, amide and/or urethane attached to the duration enhancing moiety and the peptide.

In some embodiments, the linker results from direct chemical conjugation between an amino acid side chain of a backbone functionality (moiety) of the peptide and a functionality on the duration enhancing moiety. Exemplary of this type of bonding is the formation of an amide bond achieved by standard solid-phase synthetic methods, as well known in the art. The linkers described herein are exemplary, and linkers within the scope of this invention may be much longer and may include other residues.

In some embodiments, the linker includes two or more of substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In some embodiments, the linker has the structure -L¹-L²-, wherein L¹ and L² are each independently a divalent heteroatom, —O—, —S—, —S—S—, —OCO—, —OCONH—, and —NHCONH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In some embodiments, L¹ and L² are each independently —OCO—(CH₂)ₙ—CO—, —O—(CH₂)ₙ—NHCO—, —O—(CH₂)ₙ—, —O—(CH₂)ₙ—CONH—(CH₂)ₙ—, —O—(CH₂)ₙ—, —SO₂—(CH₂)ₙ—, —SO₂—(CH₂)ₙ—, S—, wherein “n” is independently 1-5 at each occurrence.

In some embodiments, the linker has the structure —OCO—(CH₂)ₙ—CO—, —O—(CH₂)ₙ—NHCO—, —O—(CH₂)ₙ—, —O—(CH₂)ₙ—CONH—(CH₂)ₙ—, —O—(CH₂)ₙ—, —SO₂—(CH₂)ₙ—, —SO₂—(CH₂)ₙ—, S—, wherein “n” is independently 1-5 at each occurrence.

In some embodiments, a substituted group within a linker or a substituted linker group described herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted or unsubstituted cycloalkylene, substituted or unsubstituted heterocycloalkylene, substituted or unsubstituted arylene, or substituted or unsubstituted heteroarylene within a linker described herein is substituted with at least one substituent group. In other embodiments, at least one or all of these groups are substituted with at least one size-limited substituent group. Alternatively, at least one or all of these groups are substituted with at least one lower substituent group.

In other embodiments of the linkers described herein, each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, each substituted or unsubstituted heterocycloalkyl is a substituted or unsubstituted 4 to 8 membered heterocycloalkyl, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene substituted or unsubstituted C₄-C₈ cycloalkylene, and each substituted or unsubstituted heterocycloalkylene is a substituted or unsubstituted 4 to 8 membered heterocycloalkylene.

Alternatively, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, each substituted or unsubstituted heterocycloalkyl is a substituted or unsubstituted 5 to 7 membered heterocycloalkyl, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene substituted or unsubstituted C₅-C₆ cycloalkylene, and each substituted or unsubstituted heterocycloalkylene is a substituted or unsubstituted 5 to 7 membered heterocycloalkylene.

In some embodiments, a duration enhancing moiety is attached to a compound described herein via linkers known in the art, for example but not limited to, the cysteine linked PEG as shown in Formula (III) following. In the formula, “n” determines the size of the PEG conjugated to the peptide.

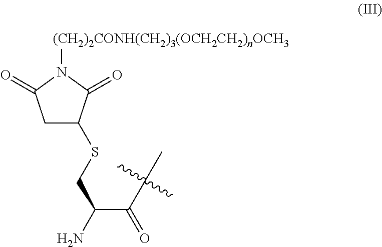

Peptides useful in the compounds and methods described herein include, but are not limited to, the peptides set forth in residues 1-32 of Formulae (I)-(II) provided in Tables 1-2 following. Unless indicated to the contrary, all peptides described herein, including peptides having an expressly provided sequence, are contemplated in both free carboxylate and amidated forms. Unless indicated to the contrary, the terms “octyl,” “decanoyl,” “lauryl,” “palmytoyl” and the like forming part of a peptide sequence name described herein (e.g., Table 1) refer to the product of acylation at the N-terminal, providing a substituted N-terminal amide. The term “Ac” refers to acetylation, typically at the N-terminal. The term “For” in the context of derivatization of a side chain amine (e.g., Lys) refers to formylation. The terms “L-Ocg” and “D-Ocg” refer to the L- and D-stereoisomers of 2-aminodecanoic acid (also known as octylglycine), respectively. The term “2NaI” refers to 2-naphtylalanine. The term “Dap” refers to diaminopropionic acid. The term “Agy” refers to allylglycine. The term “Aib” refers to aminoisobutyric acid. The term “beta-A” refers to beta-alanine. The term “homo” prepended to an amino acid name or abbreviation refers to the corresponding homolog having one less carbon atom in the side chain, e.g., homoarginine (homoR). “Hor” refers to hydroorotic acid. “Isocap” refers to isocaproyl. “Cit” refers to citrulline.

TABLE 1
peptides useful in the peptide conjugates
described herein.

| Cmpd | Description (sequence) |
|---|---|
| 1 | KCNTATCVLGRLSQELHRLQTYPRTNVGSNTY-NH₂ |
| 2 | CNTATCVLGRLSQELHRLQTYPRTNVGSNTY-NH₂ |
| | ([desLys¹]-Cmpd 1) |
| 3 | KCNTATCVLGRLSQELHRLQKYPRTNVGSNTY-NH₂ |
| 4 | Ac-CNTATCVLGKLSQELHRLQTYPRTNVGSNTY-NH₂ |
| 5 | Ac-CNTATCVLGRLSQELHKLQTYPRTNVGSNTY-NH₂ |
| 6 | Ac-CNTATCVLGRLSQELHRLQTKPRTNVGSNTY-NH₂ |
| 7 | Ac-CNTATCVLGRLSQELHRLQTYKRTNVGSNTY-NH₂ |
| 8 | Ac-CNTATCVLGRLSQELHRLQTYPKTNVGSNTY-NH₂ |
| 9 | Ac-CNTATCVLGRLSQELHRLQTYPRTNVGSNTYK-NH₂ |
| 10 | GGGCNTATCVLGRLSQELHRLQTYPRTNVGSNTY-NH₂ |
| 11 | Ac-CNTATCVLGRLSQELHRLQK(GGG)YPRTNVGSNTY-NH₂ |
| 12 | Ac-CNTATCVLGRLSQELHRLQKYPRTNVGSNTY-NH₂ |
| 13 | KCNTATCVLGRLADFLHRFHTFPRTNTGSNTY-NH₂ |
| 14 | CNTATCVLGRLADFLHRFHTFPRTNTGSNTY-NH₂ |
| 15 | Ac-CNTATCVLGRLSQELHRLQTYPRTKVGSNTY-NH₂ |
| 16 | SCNTATCVLGRLADFLHRLQTYPRTNTGSNTY-NH₂ |
| 17 | KCNTATCALQRLAQELHRLQTYPRTNVGSNTY-NH₂ |
| 18 | KCNTATCVLGRLSQELHRLQTYPRTNTGSNTY-NH₂ |
| 19 | [desamino-Cys]- |
| | NTATCVLGRLSQELHRLQKYPRTNVGSNTY-NH₂ |
| 20 | Fmoc-KCNTATCVLGRLSQELHRLQKYPRTNVGSNTY-NH₂ |
| 21 | Fmoc-KCNTATCVLGRLSQELHRLQTYPRTKVGSNTY-NH₂ |

Additional peptides contemplated for the compounds and methods described herein are provided in Table 2 following.

TABLE 2
peptides useful in the peptide conjugates
described herein.

| | |
|---|---|
| 22 | CSNLSTCVLGKLSQELHKLQTYPRTNTGSGTP-NH₂ |
| 23 | CGNLSTCMLGTYTQDFNKFHTFPQTAIGVGAP-NH₂ |
| 24 | KCNTATCVLGRLSQELHRLQTYPRTNVSEAF-NH₂ |
| 25 | KCNTATCVLGRLTEFLHRLQTYPRTNTGSNTY-NH₂ |
| 26 | KCNTATCVLGRLAAALHRLQTYPRTNTGSNTY-NH₂ |
| 27 | KCNTATCVLGRLNDLLHRLQTYPRTNTGSNTY-NH₂ |
| 28 | KCNTATCVLGRLAAFLHRLQTYPRTNTGSNTY-NH₂ |
| 29 | KCNTATCATQRLANELVRLQTYPRTNVGSNTY-NH₂ |
| 30 | KCNTATCVLGRLYDYLHRLQTYPRTNTGSNTY-NH₂ |
| 31 | KCNTATCVLGRLFDFLHRLQTYPRTNTGSNTY-NH₂ |
| 32 | KCNTATCVLGRLSQELH-Cit-LQTYPRTNTGSNTY-NH₂ |
| 33 | KDNTATKVLGRLSQELHRLQTYPRTNTGSNTY-NH₂ |
| 34 | KCDTATCVTHRLSQELHRLQTYPRTNTGSNTY-NH₂ |
| 35 | KCNTATCVLGRLADFLHRFQTFPRTNTGSGTP-NH₂ |
| 36 | SCNTATCVLGRLSQELHRLQTYPRTNTGSNTY-NH₂ |
| 37 | KCNTATCVLGRLSQELHRLQTYPRTNTGSKAF-NH₂ |
| 38 | GCNTATCQVQNLSHRLWQLRQDSAPVDPSSPHSY-NH₂ |
| 39 | CSNLSTCVLGRLSQELHRLQTYPRTNTGSNTY-NH₂ |
| 40 | KCNTATCVLGKLSQELHRLQTYPRTNTGSNTY-NH₂ |
| 41 | KCNTAACVLGRLSQELHRLQTYPRTNTGSNTY-NH₂ |
| 42 | KCNTATCVLGRLSQELHKLQTYPRTNTGSNTY-NH₂ |
| 43 | KCNTATCVLGRLSQELHRLQTYPRTNTGSGTP-NH₂ |
| 44 | CSALSTCVLGRLSQELHRLQTYPRTNTGSNTY-NH₂ |
| 45 | Isocap- |
| | KCNTATCVLGRLSQELHRLQTYPRTNTGSNTY-NH₂ |
| 46 | KCNTATCVLGRLSQELHRLQTYPRTNTGSNTY-NH₂ |
| 47 | KCNTATCVLG-Cit-LSQELHRLQTYPRTNTGSNTY-NH₂ |
| 48 | Isocap- |
| | KCNTATCVLGRLSQELHRLQTYPRTNTGSNTY4Abu-NH₂ |
| 49 | ACDTATCVLGRLSQELHRLQTYPRTNTGSNTY-NH₂ |
| 50 | KCNTATCVLGRLADALHRLQTYPRTNTGSNTY-NH₂ |
| 51 | KCNTATCVLGRLAQFLHRLQTYPRTNTGSNTY-NH₂ |
| 52 | CNTATCVLGRLADFLHRLQTYPRTNTGSNTY-NH₂ |
| 53 | SCNTATCVLGRLADALHRLQTMPRTNTGSNTY-NH₂ |
| 54 | KCNTATCVLGRLTDTLHRLQTYPRTNTGSNTY-NH₂ |
| 55 | KCNTATCVLGRLEEELHRLQTYPRTNTGSNTY-NH₂ |
| 56 | HCNTATCVLGRLEEELHRLQTYPRTNTGSNTY-NH₂ |
| 57 | FCNTATCVLGRLADFLHRLQTYPRTNTGSNTY-NH₂ |
| 58 | HGECNTATCVLGRLSQELHRLQTYPRTNTGSNT-NH₂ |
| 59 | KCNTATCLLQRLQKELQRLKQYPRTNTGSNTY-NH₂ |
| 60 | HEGCNTATCVLGRLSQELHRLQTYPRTNTGSNT-NH₂ |
| 61 | CSNLSTCATQRLANELVRLQTYPRTNVGSNTY-NH₂ |
| 62 | KCNTASCVLGRLSQELHRLQTYPRTNTGSNTY-NH₂ |
| 63 | KCNTAVCVLGRLSQELHRLQTYPRTNTGSNTY-NH₂ |
| 64 | KCNTATCVLGRLSQELHRYPRTNTGSNTY-NH₂ |
| 65 | KCNTATCVLGRLSQELYPRTNTGSNTY-NH₂ |
| 66 | KCNTATCVLGRLSQELHRLQTLQTYPRTNTGSNTY-NH₂ |
| 67 | KCNTATCVLGKLSQELHKLQTYPRTNTGSNTY-NH₂ |
| 68 | KCNTAHseCVLGRLSQELHRLQTYPRTNTGSNTY-NH₂ |
| 69 | KCNTAAhbCVLGRLSQELHRLQTYPRTNTGSNTY-NH₂ |
| 70 | STAVLGRLSQELHRLQTYPRTNTGSNTY-NH₂ |
| 71 | KCNTATCVLG-Orn-LSQELH-Orn-LQTYPRTNTGSNTY-NH₂ |
| 72 | KCNTATCVLG-Cit-LSQELH-Cit-LQTYPRTNTGSNTY-NH₂ |
| 73 | GCNTATCQVQNLSHRLWQLRQDSAPVEPSSPHSY-NH₂ |
| 74 | CNTATCVLGRLSQELHRLQTYPRTNTGSNTY-NH₂ |
| 75 | KCNTATCVLG-homoR-LSQELH-homoR-LQTYPRTNTGSNTY- |
| | NH₂ |
| 76 | FD-(beta-A)-(beta- |
| | A)CNTATCVLGRLSQELHRLQTYPRTNTGSNTY-NH₂ |
| 77 | CSNLSTCVLGKLSQELHKLQTYPRTNTGSGTP-NH₂ |
| 78 | SSNLSTSATQRLANELVRLQTYPRTNVGSNTY-NH₂ |
| 79 | LSTCVLGRLSQELHRLQTYPRTNTGSNTY-NH₂ |
| 80 | AcLSTSVLGRLSQELHRLQTYPRTNTGSNTY-NH₂ |
| 81 | AcVLGKLSQELNKFHTFPQTAIGVGAP-NH₂ |
| 82 | AcATQRLANFLVRSSNNLGPVLPPTNVGSNTY-NH₂ |
| 83 | LSTSVLGRLSQELHRLQTYPRTNTGSNTY-NH₂ |
| 84 | Ac-LSTAVLGRLSQELHRLQTYPRTNTGSNTY-NH₂ |
| 85 | KCNTATCATQRLANFLVHSSNNGY-NH₂ |
| 86 | KCNTATCALQRLAQELHRLQALPRTNVGSNTY-NH₂ |
| 87 | KCNTATCALQRLSQELHRLQALPRTNVGSNTY-NH₂ |
| 88 | KCNTATCVLGRLAQELHRLQALPRTNVGSNTY-NH₂ |
| 89 | Ocg-KCNTATCVLGRLSQELHRLQTYPRTNTGSNTY-NH₂ |
| 90 | KCNTATCVLGRLSQELHRLQALPRTNVGSNTY-NH₂ |
| 91 | KCNTATCVLGRLAQELHRLQTYPRTNVGSNTY-NH₂ |
| 92 | KCNTATCALQRLSQELHRLQTYPRTNVGSNTY-NH₂ |
| 93 | K(L-Hor)CNTATCVLGRLSQELHRLQTYPRTNVGSNTY-NH₂ |
| 94 | K(D-Hor)CNTATCVLGRLSQELHRLQTYPRTNVGSNTY-NH₂ |
| 95 | KCNTATCVLGRLSQELHK(L-Hor)LQTYPRTNVGSNTY-NH₂ |
| 96 | KCNTATCVLGRLSQELHK(D-Hor)LQTYPRTNVGSNTY-NH₂ |
| 97 | KCNTATC-Aib-LQRLSQELHRLQTYPRTNVGSNTY-NH₂ |
| 98 | KCNTATCVLGRL-Aib-QELHRLQTYPRTNVGSNTY-NH₂ |
| 99 | KCNTATCAibLQRL-Aib-QELHRLQTYPRTNVGSNTY-NH₂ |
| 100 | KCNTATCVLERLKQELHRLQTYPRTNVGSNTY-NH₂ |
| 101 | KCNTATCVLGRLSQELHKLQTYPRTNVGSNTY-NH₂ |
| 102 | KCNTATCVLGKLSQELHRLQTYPRTNVGSNTY-NH₂ |
| 103 | KCNTATCVLGRLSQELERLQKYPRTNVGSNTY-NH₂ |
| 104 | KCNTATCVLERLKQELHRLQTYPRTNVGSNTY-NH₂ |
| 105 | KCNTATCVLGRLSQELERLKTYPRTNVGSNTY-NH₂ |
| 106 | KCNTATCVLERLSKELHRLQTYPRTNVGSNTY-NH₂ |
| 107 | KCNTATCVLGKLSQELHRLQTYPRTNVGSNTY-NH₂ |
| 108 | KCNTATCVLGRLSQELERLQKYPRTNVGSNTY-NH₂ |
| 109 | KCNTATCVLERLSKELHRLQTYPRTNVGSNTY-NH₂ |
| 110 | GAPPPSKCNTATCVLGRLSQELHRLQTYPRTNVGSNTY-NH₂ |
| 111 | KCNTATCVLGRLSQELERLKTYPRTNVGSNTY-NH₂ |
| 112 | KCNTATCVLGRLSQELHKLQTYPRTNVGSNTY-NH₂ |
| 113 | KCNTATCALQRLADFLHRLQTYPRTNVGSNTY-NH₂ |
| 114 | Ocg-KCNTATCALQRLAQELHRLQTYPRTNVGSNTY-NH₂ |
| 115 | KCNTATCALQRLAQELHK(L-Hor)LQTYPRTNVGSNTY-NH₂ |
| 116 | KCNTATCVLGRLSQELHRLQHYPRTNVGSNTY-NH₂ |
| 117 | KCNTATCVLGRLHQELHRLQTYPRTNVGSNTY-NH₂ |
| 118 | KCNTATCVLGRLSQELHRLQTYARTNVGSNTY-NH₂ |
| 119 | KCNTATCVLGRLSQELHRLQTYPATNVGSNTY-NH₂ |
| 120 | KCNTATCVLGRLSQELHRLQTYPRANVGSNTY-NH₂ |
| 121 | KCNTATCVLGRLSQELHRLQTYPRTAVGSNTY-NH₂ |
| 122 | KCNTATCVLGRLSQELHRLQTYPRTNAGSNTY-NH₂ |
| 123 | KCNTATCVLGRLSQELHRLQTYPRTNVASNTY-NH₂ |
| 124 | KCNTATCVLGRLSQELHRLQTYPRTNVGANTY-NH₂ |
| 125 | KCNTATCVLGRLSQELHRLQTYPRTNVGSATY-NH₂ |
| 126 | KCNTATCVLGRLSQELHRLQTYPRTNVGSNAY-NH₂ |
| 127 | KCNTATCVLGRLSQELHRLQTYPRTNVGSNTA-NH₂ |
| 128 | KCNTATCVLGRLSQELHALQTYPRTNVGSNTY-NH₂ |
| 129 | KCNTATCVLGRLSQELHRAQTYPRTNVGSNTY-NH₂ |
| 130 | KCNTATCVLGRLSQELHRLATYPRTNVGSNTY-NH₂ |
| 131 | KCNTATCVLGRLSQELHRLQAYPRTNVGSNTY-NH₂ |
| 132 | KCNTATCVLGRLSQELHRLQTAPRTNVGSNTY-NH₂ |
| 133 | KCNTATCVLGRLSQALHRLQTYPRTNVGSNTY-NH₂ |
| 134 | KCNTATCVLGRLSQEAHRLQTYPRTNVGSNTY-NH₂ |
| 135 | KCNTATCVLGRLSQELARLQTYPRTNVGSNTY-NH₂ |
| 136 | SCNTATCVLGRLADALHRLQTLPRTNTGSNTY-NH₂ |
| 137 | SCNTATCVLGRLAEALHRLQTLPRTNTGSNTY-NH₂ |
| 138 | SCNTATCVLGRLEEALHRLQTLPRTNTGSNTY-NH₂ |
| 139 | SCNTATCALQRLADALHRLQTLPRTNTGSNTY-NH₂ |
| 140 | SCNTATCALQRLAEALHRLQTLPRTNTGSNTY-NH₂ |
| 141 | KCNTATCVLGRLSQELHRAQTLQTYPRTNTGSNTY-NH₂ |
| 142 | KCNTATCVLGRLSQELHRLQTLQTYPRTNVGSNTY-NH₂ |
| 143 | KCNTATCVLGRLSQELHRAQTLQTYPRTNVGSNTY-NH₂ |
| 144 | KCNTATCVLGRLADALHRLQTLQTYPRTNTGSNTY-NH₂ |
| 145 | KCNTATCIDLTFHLLRTLLELAPRTNTGSNTY-NH₂ |
| 146 | KCNTATCIDLTFHLLRTLLELARTQSQPRTNTGSNTY-NH₂ |
| 147 | DNPSLSVLGRLSQELHRLQTYAEQNRIIFDSV-NH₂ |
| 148 | KCNTATCVLGRLSQELHRLQTYRTQSQRERAEQNRIIFDSV-NH₂ |
| 149 | DNPSLSIDLTFHLLRTLLELAPRTNTGSNTY-NH₂ |
| 150 | Ac-SCNTATCVLGRLAEALHRLQKLPRTNTGSNTY-NH₂ |

In some embodiments, the duration enhancing moiety is included within a “linked duration enhancing moiety” with formula -L-R, wherein R is a duration enhancing moiety as described herein, and L is a linker or a bond. Where L is a linker, L can be —C(O)—, —NH—, —O—, —S—, —S—S—, —OCO—, —OCONH—, —NHCONH—, substituted or unsubstituted alkylene, substituted or unsubstituted alkenylene, substituted or unsubstituted urethane, substituted or unsubstituted alkylamide, substituted or unsubstituted alkylsulfone, substituted or unsubstituted heteroalkylene, substituted or unsubstituted cycloalkylene, substituted or unsubstituted heterocycloalkylene, substituted or unsubstituted arylene, or substituted or unsubstituted heteroarylene, and the like, as known in the art.

In some embodiments, L is R¹-substituted or unsubstituted alkylene, R¹-substituted or unsubstituted alkenylene, R¹-substituted or unsubstituted urethane, R¹-substituted or unsubstituted alkylamide, R¹-substituted or unsubstituted alkylsulfone, R¹-substituted or unsubstituted heteroalkylene, R¹-substituted or unsubstituted cycloalkylene, R¹-substituted or unsubstituted heterocycloalkylene, substituted or unsubstituted arylene, or substituted or unsubstituted heteroarylene. R¹ is R²-substituted or unsubstituted alkyl, R²-substituted or unsubstituted heteroalkyl, R²-substituted or unsubstituted cycloalkyl, R²-substituted or unsubstituted heterocycloalkyl, R²-substituted or unsubstituted aryl, or R²-substituted or unsubstituted heteroaryl. R² is R³-substituted or unsubstituted alkyl, R³-substituted or unsubstituted heteroalkyl, R³-substituted or unsubstituted cycloalkyl, R³-substituted or unsubstituted heterocycloalkyl, R³-substituted or unsubstituted aryl, or R³-substituted or unsubstituted heteroaryl. R³ is unsubstituted alkyl, unsubstituted heteroalkyl, unsubstituted cycloalkyl, unsubstituted heterocycloalkyl, unsubstituted aryl or unsubstituted heteroaryl.

In some embodiments, the linked duration enhancing moiety -L-R is covalently bonded to an amino acid side chain of the peptide, or to a backbone atom or moiety thereof. Exemplary backbone moieties include a free amine at the N-terminal, and a free carboxyl or carboxylate at the C-terminal. In some embodiments, an amino acid side chain or a backbone atom or moiety is covalently bonded to a polyethylene glycol, a long chain aliphatic group, or a derivative thereof.

In some embodiments, the duration enhancing moiety R is a water-soluble polymer. A “water soluble polymer” means a polymer which is sufficiently soluble in water under physiologic conditions of e.g., temperature, ionic concentration and the like, as known in the art, to be useful for the methods described herein. A water soluble polymer can increase the solubility of a peptide or other biomolecule to which such water soluble polymer is attached. Indeed, such attachment has been proposed as a means for improving the circulating life, water solubility and/or antigenicity of administered proteins, in vivo. See, e.g., U.S. Pat. No. 4,179,337; U.S. Published Appl. No. 2008/0032408. Many different water-soluble polymers and attachment chemistries have been used towards this goal, such as polyethylene glycol, copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and the like.

In some embodiments, the linked duration enhancing moiety -L-R includes a polyethylene glycol. Polyethylene glycol (“PEG”) has been used in efforts to obtain therapeutically usable peptides. See, e.g., Zalipsky, S., 1995, Bioconjugate Chemistry 6:150-165; Mehvar, R., 2000, J. Pharm. Pharmaceut. Sci. 3:125-136. As appreciated by one of skill in the art, the PEG backbone [(CH₂CH₂—O—)ₙ, n: number of repeating monomers] is flexible and amphiphilic. Without wishing to be bound by any theory or mechanism of action, the long, chain-like PEG molecule or moiety is believed to be heavily hydrated and in rapid motion when in an aqueous medium. This rapid motion is believed to cause the PEG to sweep out a large volume and prevents the approach and interference of other molecules. As a result, when attached to another chemical entity (such as a peptide), PEG polymer chains can protect such chemical entity from immune response and other clearance mechanisms. As a result, pegylation can lead to improved drug efficacy and safety by optimizing pharmacokinetics, increasing bioavailability, and decreasing immunogenicity and dosing frequency. “Pegylation” refers to conjugation of a PEG moiety with another compound. For example, attachment of PEG has been shown to protect proteins against proteolysis. See, e.g., Blomhoff, H. K. et al., 1983, Biochim Biophys Acta 757:202-208. Unless expressly indicated to the contrary, the terms “PEG,” “polyethylene glycol polymer” and the like refer to polyethylene glycol polymer and derivatives thereof, including methoxy-PEG (mPEG).

Methods for attaching polymer moieties, such as PEG and related polymers, to reactive groups found on a peptides and proteins are well known in the art. Typical attachment sites in proteins include primary amino groups, such as those on lysine residues or at the N-terminus, thiol groups, such as those on cysteine side-chains, and carboxyl groups, such as those on glutamate or aspartate residues or at the C-terminus. Common sites of attachment are to the sugar residues of glycoproteins, cysteines or to the N-terminus and lysines of the target peptide. The terms “pegylated” and the like refer to covalent attachment of polyethylene glycol to a peptide or other biomolecule, optionally through a linker as described herein and/or as known in the art.

In some embodiments, a PEG moiety in a peptide conjugate described herein has a nominal molecular weight within a specified range. The size of a PEG moiety is indicated by reference to the nominal molecular weight, typically provided in kilodaltons (kD). The molecular weight is calculated in a variety of ways known in the art, including number, weight, viscosity and “Z” average molecular weight. It is understood that polymers, such as PEG and the like, exist as a distribution of molecule weights about a nominal average value.

Exemplary of the terminology for molecular weight for PEGs, the term “mPEG40KD” refers to a methoxy polyethylene glycol polymer having a nominal molecular weight of 40 kilodaltons. Reference to PEGs of other molecular weights follows this convention. In some embodiments, the PEG moiety has a nominal molecular weight in the range 10-100 KD, 20-80 KD, 20-60 KD, or 20-40 KD. In some embodiments, the PEG moiety has a nominal molecular weight of 10, 15, 20, 25, 30, 35, 40, 45, 50, 55, 60, 65, 70, 75, 80, 85, 90, 95 or even 100 KD. Preferably, the PEG moiety has a molecular weight of 20, 25, 30, 40, 60 or 80 KD.

PEG molecules useful for derivatization of peptides are typically classified into linear, branched and Warwick (i.e., PolyPEG®) classes of PEGs, as known in the art. Unless expressly indicated to the contrary, the PEG moieties described herein are linear PEGs. Furthermore, the terms “two arm branched,” “Y-shaped” and the like refer to branched PEG moieties, as known in the art. The term “Warwick” in the context of PEGs, also known as “comb” or “comb-type” PEGs, refers to a variety of multi-arm PEGs attached to a backbone, typically poly(methacrylate), as known in the art. Regarding nomenclature including conventions employed in the table provided herein, absent indication to the contrary a PEG moiety is attached to the backbone of the peptide. For example, Cmpd 151 is the result of the conjugation of mPEG40 KD to the N-terminal nitrogen of Cmpd 2. Similarly, Cmpd 160 is the result of conjugation of a two arm branched mPEG40 KD with Cmpd 2. N-terminal acetylation of the peptide is indicated with the term “Acetyl” or “Ac.” Substitutions at specific residues are indicated within square brackets. Standard single letter abbreviations for amino acids can be used, as can standard three-letter abbreviations. For example, Cmpd 169 is an N-terminal acetylated analogs of Cmpd 2 wherein the residue at position 26 of Cmpd 2 is substituted for lysine, and the pendant amine functionality of lysine 26 (i.e., K²⁶) is conjugated with a PEG40 KD moiety. Exemplary compounds are provided in Table 3 following.

TABLE 3
Pegylated compounds

| Cmpd | Description |
|---|---|
| 151 | mPEG40KD-Cmpd 2 |
| 152 | mPEG20KD-Cmpd 2 |
| 153 | mPEG30KD-Cmpd 2 |
| 154 | mPEG40KD-CH₂NH-Cmpd 2 |
| 155 | mPEG40KD-CH₂NH-Cmpd 1 |
| 156 | Acetyl-[K²¹(mPEG40KD)]-Cmpd 2 |
| 157 | mPEG40KD-GGG-Cmpd 2 |
| 158 | mPEG4K0D-Cmpd 16 |
| 159 | mPEG40KD-desLys¹-Cmpd 13 |
| 160 | Two arm Branched mPEG40KD-Cmpd 2 |
| 161 | Two arm Branched mPEG60KD-Cmpd 2 |
| 162 | Two arm Branched mPEG80KD-Cmpd 2 |
| 163 | Warwick PEG25KD-CH₂NH-Cmpd 2 |
| 164 | Warwick PEG40KD-CH2NH-Cmpd 2 |
| 165 | Warwick PEG60KD-CH2NH-Cmpd 2 |
| 166 | Warwick PEG25KD-Cmpd 2 |
| 167 | Warwick PEG40KD-Cmpd 2 |
| 168 | Warwick PEG60KD-Cmpd 2 |
| 169 | Acetyl-[K²⁶(mPEG40KD)]-Cmpd 2 |
| 170 | Acetyl-[K²²(mPEG40KD)]-Cmpd 2 |
| 171 | Acetyl-[K²³(mPEG40KD)]-Cmpd 2 |
| 172 | Acetyl-[K¹¹(mPEG40KD)]-Cmpd 2 |
| 173 | Acetyl-[K¹⁸(mPEG40KD)]-Cmpd 2 |
| 174 | Acetyl-[K24(mPEG40KD)]-Cmpd 2 |
| 175 | [K²¹(mPEG40KD)]-desLys1-desaminoCys2-Cmpd 1 |
| 176 | [K²¹(mPEG40KD)]-Cmpd 2 |
| 177 | Acetyl-[K²¹(GGG-mPEG40KD)]-Cmpd 2 |
| 178 | Acetyl-[K²¹(CH₂NH-mPEG40KD)]-Cmpd 2 |
| 179 | Acetyl-[K²⁶(Y-shaped-mPEG40KD)]-Cmpd 2 |
| 180 | Acetyl-[K²⁶(CH₂NH-mPEG40KD)]-Cmpd 2 |
| 181 | Acetyl-[K²⁶(Two-arm branched mPEG40KD)]-Cmpd 2 |
| 182 | Acetyl-[K²⁶(Two-arm branched mPEG80KD)]-Cmpd 2 |
| 183 | Acetyl-[K²⁶(NHCOO-mPEG40KD)]-Cmpd 2 |

Additional compounds described herein having PEG moieties with molecular weight in the range 1-20, 1-10, or even 1-5 KD are disclosed in Table 4 following.

TABLE 4
Pegylated compounds having PEGs in the range 1-20 KD.

| Cmpd | Description |
|---|---|
| 184 | [K¹⁸(mPEG2KD)]-Cmpd 1 |
| 185 | Acetyl-[K^{18,21}(mPEG5KD)]-Cmpd 18 |

In some embodiments, the linked duration enhancing moiety -L-R includes a long chain aliphatic group, and the resulting compound is a long chain peptide conjugate. Accordingly, the term “long chain peptide conjugate” as used herein refers to a peptide to which a long chain aliphatic group is attached, optionally through a linker. Thus, a further strategy for modulating the duration of activity and potency of peptide and protein therapeutic agents involves derivatizing with long chain aliphatic (e.g., fatty acid) chains of various lengths, for example but not limited to C₆-C₂₄, C₈-C₂₀, C₁₀-C₁₈, C₁₂-C₁₆, and the like. A “fatty acid” as used herein means a long chain aliphatic moiety terminated with a carboxyl functionality. It is understood that long chain aliphatic groups can be fully hydrogenated or partially dehydrogenated. The term “Cₛ” (e.g., C₆, C₈, and the like) refers to a carbon chain containing “x” carbon atoms. In some embodiments, the carboxyl functionality of a fatty acid is available for bonding with the peptide. Indeed, the acylation of amino groups is a common means employed for chemically modifying proteins, and general methods of acylation are known in the art and include the use of activated esters, acid halides, or acid anhydrides. See, e.g., Methods of Enzymology 25:494-499 (1972), U.S. Pat. No. 7,402,565 and RE37,971, each of which is incorporated herein by reference in its entirety and for all purposes. Such long chain conjugation may occur singularly at the N- or C-terminus or at the side chains of amino acid residues within the sequence of the peptide. Linkers may be employed between the long chain aliphatic groups or fatty acid groups and the peptide, as known in the art and described herein. There may be multiple sites available for bonding along the peptide. Substitution of one or more amino acids with lysine, aspartic acid, glutamic acid, or cysteine may provide additional sites for bonding. See, e.g., U.S. Pat. Nos. 5,824,784 and 5,824,778. Fatty acid chain(s) may be linked to an amino, carboxyl, or thiol group, and may be linked by N or C terminus, or at the side chains of lysine, aspartic acid, glutamic acid, or cysteine, as known in the art and/or as described herein. The fatty acid moieties may be linked with diamine and dicarboxylic groups, as known in the art. Additional strategies for incorporation of fatty acid chains are known in the art and/or described herein.

Methods for conjugation of long chain (e.g., C₆-C₂₄) aliphatic groups, preferably fatty acid chains, to peptides are available to the skilled artisan. Compounds having enhanced duration of action and which are beneficial in the treatment of psychiatric diseases or disorders include the compounds of Table 5 following. In some embodiments, the long chain aliphatic group is C₁₆, C₁₈, C₂₀, C₂₂ or even C₂₄. In some embodiments, the long chain aliphatic group is fully hydrogenated. In some embodiments, the long chain aliphatic group contains one or more double bonds.

TABLE 5
Long chain acylated compounds

| Cmpd | Description |
|---|---|
| 186 | [K¹⁸ε-(γ-Glu(N_{α}-C₁₆-Chain))]-Cmpd 17 |
| 187 | [K²¹ε-(γ-Glu(N_{α}-C₁₆-Chain))]-Cmpd 17 |
| 188 | [K²⁴ε-(γ-Glu(N_{α}-C₁₆-Chain))]-Cmpd 17 |
| 189 | [K²⁶ε-(γ-Glu(N_{α}-C₁₆-Chain))]-Cmpd 17 |
| 190 | [K¹⁸ε-(γ-D-Glu(N_{α}-C₁₆-Chain))]-Cmpd 17 |
| 191 | [K¹⁸ε-(γ-Glu(N_{α}-C₁₈-Chain))]-Cmpd 17 |
| 192 | [K¹ε-(γ-Glu(N_{α}-C₁₆-Chain))]-Cmpd 17 |
| 193 | [K¹¹ε(γ-Glu(N_{α}-C₁₆-Chain))]-Cmpd 17 |
| 194 | [K¹⁸ε-(g-Glu(N_{α}-C₂₂-Chain))]-Cmpd 17 |
| 195 | Kε-(γ-Glu(N_{α}-C₁₆-Chain))-Cmpd 17 |

In some embodiments, the duration enhancing moiety is attached to the side chain of a peptide with sequence according to any of Formulae (I)-(II) at residue 11, 18, 21, 22, 23, 24 or 26.

In some embodiments, the duration enhancing moiety -L-R conjugated with a peptide described herein includes an unstructured recombinant peptide. See e.g., Schellenberger et al., 2009, Nature Biotechnology 27:1186-1192, incorporated herein by reference and for all purposes. The terms “recombinant PEG,” “rPEG,” “rPEG duration enhancing moiety” and the like refer to substantially unstructured recombinant peptide sequences which act as surrogates for PEG as duration enhancing moieties in conjugation with peptides having a defined sequence identity relative to the amino acid sequence of Formulae (I)-(II). rPEGs and peptide conjugates thereof have the potentially significant advantage that synthesis can be achieved by recombinant methods, not requiring the solid-phase or solution-phase chemical synthetic steps of, for example but not limited to, conjugation of PEG with the peptide.

It has been found that stable, highly expressed, unstructured peptides can be conjugated with biologically active molecules, which results in modulation of a variety of biological parameters, including but not limited to, serum half-life. For example, by exclusively incorporating A, E, G, P, S and T, Schellenberger et al. (Id.) disclose that the apparent half-lives of conjugates with exenatide, green fluorescent protein (GFP) and human growth hormone (hGH) are significantly increased relative to the unconjugated peptides.

In some embodiments, the rPEG duration enhancing moiety does not include a hydrophobic residue (e.g., F, I, L, M, V, W or Y), a side chain amide-containing residue (e.g., N or Q) or a positively charged side chain residue (e.g., H, K or R). In some embodiments, the rPEG duration enhancing moiety includes A, E, G, P, S or T. In some embodiments, the rPEG includes glycine at 10-20%, 20-30%, 30-40%, 40-50%, 50-60%, 60-70%, 70-80%, 80-90%, 90-99%, or even glycine at 100%.

In embodiments where the rPEG duration enhancing moiety is conjugated at the N-terminal or C-terminal of the peptide which is at least 75% identical to the structure of Formula (I), the conjugated peptide and rPEG are synthesized by recombinant methods known in the art. In embodiments where the rPEG duration enhancing moiety is conjugated at a side chain of the peptide which is at least 75% identical to the structure of Formula (I), the rPEG moiety is synthesized by recombinant methods and subsequently conjugated to the peptide by methods known in the art and disclosed herein.

In some embodiments, each substituted group in a peptide conjugate described herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted or unsubstituted cycloalkylene, substituted or unsubstituted heterocycloalkylene, substituted or unsubstituted arylene, or substituted or unsubstituted heteroarylene described herein is substituted with at least one substituent group. In some embodiments, at least one or all of these groups are substituted with at least one size-limited substituent group. In some embodiments, at least one or all of these groups are substituted with at least one lower substituent group.

In some embodiments, each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, each substituted or unsubstituted heterocycloalkyl is a substituted or unsubstituted 4 to 8 membered heterocycloalkyl, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene substituted or unsubstituted C₄-C₈ cycloalkylene, and each substituted or unsubstituted heterocycloalkylene is a substituted or unsubstituted 4 to 8 membered heterocycloalkylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, each substituted or unsubstituted heterocycloalkyl is a substituted or unsubstituted 5 to 7 membered heterocycloalkyl, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene substituted or unsubstituted C₅-C₆ cycloalkylene, and each substituted or unsubstituted heterocycloalkylene is a substituted or unsubstituted 5 to 7 membered heterocycloalkylene.

The peptides of the peptide conjugates described herein may be prepared using biological, chemical, and/or recombinant DNA techniques that are known in the art. Exemplary methods are described herein and in U.S. Pat. No. 6,872,700; WO 2007/139941; WO 2007/140284; WO 2008/082274; WO 2009/011544; and US Publication No. 2007/0238669, the disclosures of which are incorporated herein by reference in their entireties and for all purposes. Other methods for preparing the compounds are set forth herein and/or known in the art.

For example, the peptides of the compounds described herein may be prepared using standard solid-phase peptide synthesis techniques, such as an automated or semiautomated peptide synthesizer. Typically, using such techniques, an alpha-N-carbamoyl protected amino acid and an amino acid attached to the growing peptide chain on a resin are coupled at room temperature in an inert solvent (e.g., dimethylformamide, N-methylpyrrolidinone, methylene chloride, and the like) in the presence of coupling agents (e.g., dicyclohexylcarbodiimide, 1-hydroxybenzo-triazole, and the like) in the presence of a base (e.g., diisopropylethylamine, and the like). The alpha-N-carbamoyl protecting group is removed from the resulting peptide-resin using a reagent (e.g., trifluoroacetic acid, piperidine, and the like) and the coupling reaction repeated with the next desired N-protected amino acid to be added to the peptide chain. Suitable N-protecting groups are well known in the art, such as t-butyloxycarbonyl (tBoc) fluorenylmethoxycarbonyl (Fmoc), and the like. The solvents, amino acid derivatives and 4-methylbenzhydryl-amine resin used in the peptide synthesizer may be purchased from a variety of commercial sources, including for example Applied Biosystems Inc. (Foster City, Calif.).

For chemical synthesis solid phase peptide synthesis can be used for the peptide conjugates, since in general solid phase synthesis is a straightforward approach with excellent scalability to commercial scale, and is generally compatible with relatively long peptide conjugates. Solid phase peptide synthesis may be carried out with an automatic peptide synthesizer (Model 430A, Applied Biosystems Inc., Foster City, Calif.) using the NMP/HOBt (Option 1) system and tBoc or Fmoc chemistry (See Applied Biosystems User's Manual for the ABI 430A Peptide Synthesizer, Version 1.3B Jul. 1, 1988, section 6, pp. 49-70, Applied Biosystems, Inc., Foster City, Calif.) with capping. Boc-peptide-resins may be cleaved with HF (−5° C. to 0° C., 1 hour). The peptide may be extracted from the resin with alternating water and acetic acid, and the filtrates lyophilized. The Fmoc-peptide resins may be cleaved according to standard methods (e.g., Introduction to Cleavage Techniques, Applied Biosystems, Inc., 1990, pp. 6-12). Peptides may be also be assembled using an Advanced Chem Tech Synthesizer (Model MPS 350, Louisville, Ky.).

Covalent attachment of PEG can be conveniently achieved by a variety of methods available to one skilled in the synthetic chemical arts. For pegylation at backbone or side chain amine, PEG reagents are typically reacted under mild conditions to afford the pegylated compound. Optionally, additional steps including but not limited to reduction are employed. In a typical peptide-mPEG conjugation scheme, N-hydroxylsuccinimide (NHS) functionalized mPEG can be mixed with peptide having a free amine in a suitable solvent (e.g., dry DMF) under nitrogen in the presence of DIPEA (e.g., 3 equivalents per TFA counterion) for a suitable time (e.g., 24 hrs). The conjugate can be precipitated by the addition of a precipitation reagent (e.g., cold diethyl ether). The precipitate can be isolated by centrifugation and dissolved in water followed by lyophilization. Purification can be afforded by a variety of chromatographic procedures (e.g., MacroCap SP cation exchange column using gradient 0.5 M NaCl). Purity can be checked by SDS-PAGE. Mass spectrometry (e.g., MALDI) can be used to characterize the conjugate after dialysis against water.

PEG-SS (succinimidyl succinate). PEG-SS reacts with amine groups under mild conditions to form the amide, as shown in Scheme 1. NHS functionalization provides amino reactive PEG derivatives that can react with primary amine groups at pH 7-9 to form stable amide bonds. Reaction can be finished in 1 hour or even less time. Exemplary reactions follow in Schemes 1 and 2.

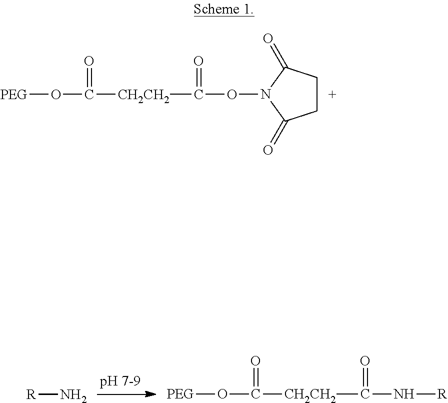

PEG-SG (succinimidyl glutarate). Similarly, PEG-SG reacts with amine groups to form the corresponding amide, as shown in Scheme 2.

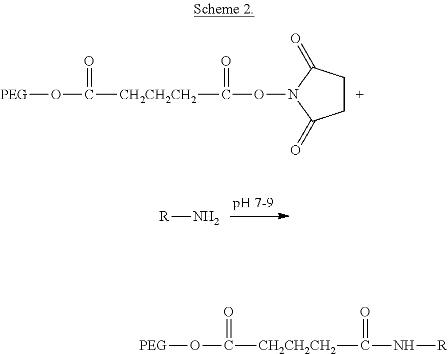

PEG-NPC (p-nitrophenyl carbonate). PEG-NPC reacts with amine functionalities to form the relatively stable urethane functionality, as shown in Scheme 3.

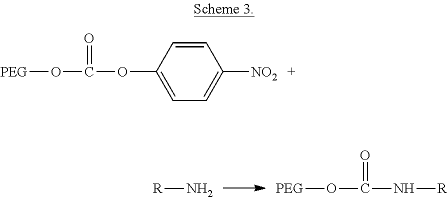

PEG-isocyanate. As shown in Scheme 4, PEG-isocyanate can react with amine to form the resultant relatively stable urethane linkage.

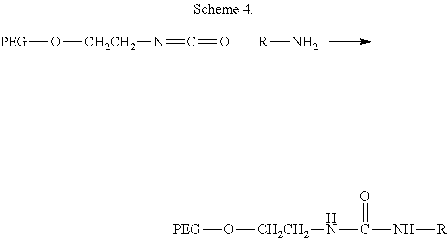

PEG-aldehyde. A variety of PEG-aldehyde reactions with amine can afford the imine, which can be further reduced to afford the pegylated amine. The reaction pH may be important for target selectivity. N-terminal amine pegylation may be at around pH 5. For example, reaction of mPEG-propionaldehyde with peptide amine, followed by reduction affords the compound depicted in Scheme 5 following.

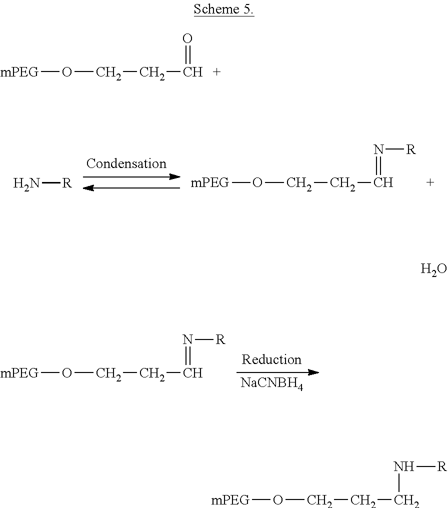

Similarly, condensation of mPEG-amide-propionaldehyde with amine and subsequent reduction can afford the compounds depicted in Scheme 6 following.

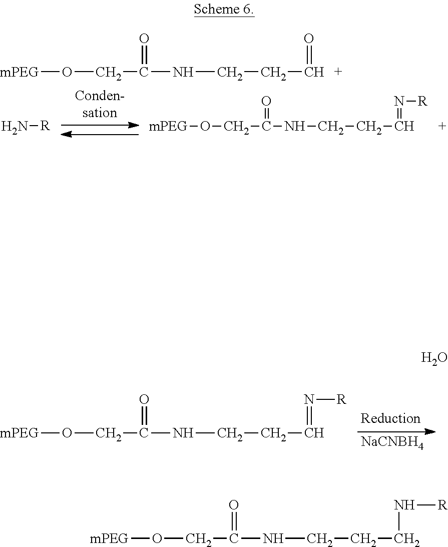

Reaction of mPEG-urethane-propionaldehyde with amine and subsequent reduction can afford the compounds depicted in Scheme 7 following.

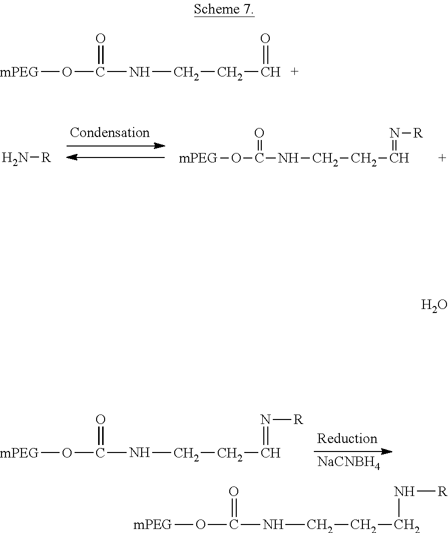

Furthermore, reaction of mPEG-butylaldehyde with amine and subsequent reduction can afford the compounds depicted in Scheme 8 following.

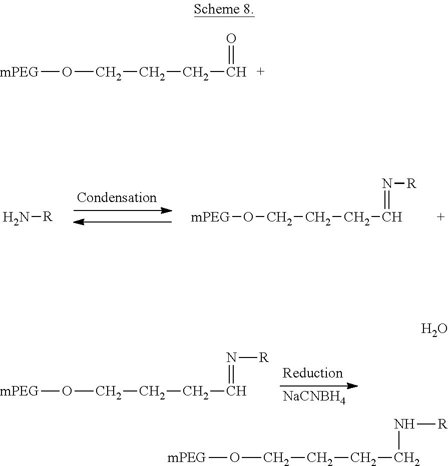

Thiol pegylation: PEG-maleimide. Pegylation is conveniently achieved at free thiol groups by a variety of methods known in the art. For example, as shown in Scheme 9 following, PEG-maleimide pegylates thiols of the target compound in which the double bond of the maleimic ring breaks to connect with the thiol. The rate of reaction is pH dependent and best conditions are found around pH 8.

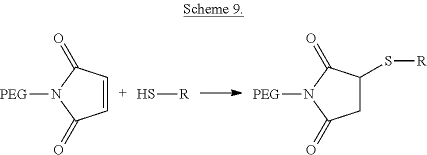

PEG-vinylsulfone. Additionally, as depicted in Scheme 10 following, PEG-vinylsulfone is useful for the pegylation of free thiol.

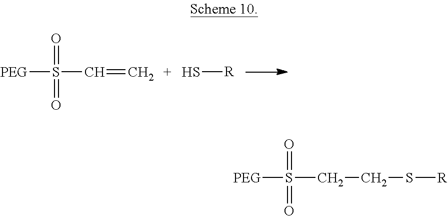

PEG-orthopyridyl-disulfide (OPSS). Formation of disulfide linked PEG to a peptide is achieved by a variety of methods known in the art, including the reaction depicted in Scheme 11 following. In this type of linkage, the resulting PEG conjugate can be decoupled from the peptide by reduction with, for example but not limited to, borohydride, small molecule dithiol (e.g., dithioerythritol) and the like.

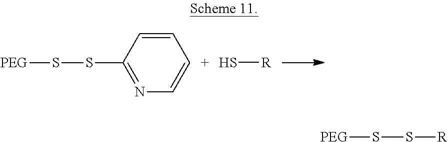

PEG-iodoacetamide. PEG-iodoacetamide pegylates thiols to form stable thioether bonds in mild basic media. This type of conjugation presents an interesting aspect in that by strong acid analysis the pegylated cysteine residue of the protein can give rise to carboxymethylcysteine which can be evaluated by a standard amino acid analysis (for example, amino acid sequencing), thus offering a method to verify the occurrence of the reaction. A typical reaction scheme is depicted in Scheme 12 following.

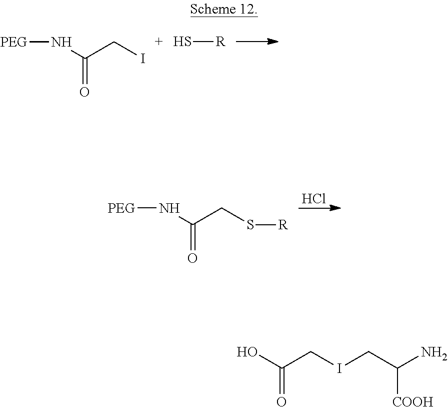

Fatty acid conjugation. Methods for the conjugation of long chain aliphatic (e.g., fatty acid) moieties are readily available to the skilled artisan.

Purification of compounds described herein generally follows methods available to the skilled artisan. In a typical purification procedure, a crude peptide-PEG conjugate is initially purified via ion exchange chromatography, e.g., Macro Cap SP cation exchanger column. A typical purification procedure employs Buffer A (20 mM sodium acetate buffer, pH 5.0) and Buffer B (20 mM sodium acetate buffer, pH 5.0, 0.5 M sodium chloride) in a gradient elution program, e.g., 0-0% Buffer B (20 min), followed by 0-50% Buffer B (50 min), then 100% Buffer B (20 min). The flow rate is typically 3 mL/min. SDS polyacrylamide gel visualization of the collected fractions is conducted, followed by dialysis against water of the suitable fraction pool and lyophilization of the resultant. Analytical characterization typically employs MALDI mass spectroscopy.

In one aspect, there is provided a method for the treatment of a psychiatric disease or disorder. The method includes administering to a patient in need of treatment an effective amount of a compound or pharmaceutical composition described herein.

As demonstrated herein, the compounds of the invention have been shown to have activity in treating psychiatric disorders. Psychiatric diseases and disorders are described in a variety of resources including the American Psychiatric Association's Diagnostic and Statistical Manual of Mental Disorders, or DSM-IV, incorporated by referenced herein and for all purposes. Broad categories of psychiatric (i.e., mental) disorders include, but are not limited to, mood disorders, anxiety disorders, schizophrenia and other psychotic disorders, substance-related disorders, sleep disorders, somatoform disorders, and eating disorders. In 2001, the National Institute of Mental Health published a summary of statistics describing the prevalence of mental disorders in America. In the report, it estimated that 22.1% of Americans ages 18 and older suffer from a diagnosable mental disorder in a given year. See Reiger et al., 1993, Archives of General Psychiatry 50:85-94). These are debilitating illnesses that affect millions of people and involve astronomical costs, in terms of treatment, lost productivity, and emotional toll.

Exemplary mood disorders include bipolar disorder and depression. Depressive disorders can encompass, among others illnesses, major depressive disorder, dysthymic disorder and bipolar disorder. About 9 to 9.5 percent of the U.S. population ages 18 and older have a depressive condition. It has been reported that the direct cost of depressive disorders is about $80 billion, with two-thirds of it being borne by businesses. The indirect costs associated with depressive disorders, such as lost productivity, are harder to calculate because of events such as “presenteeism,” described as people at work but limited in their ability to produce or participate (Durso, Employee Benefit News, December 2004). In some embodiments, a patient is treated for bipolar disorder. In some embodiments, a patient is treated for depression.

Further exemplary psychiatric conditions include anxiety disorders. These disorders can include panic disorder, obsessive-compulsive disorder, post-traumatic stress disorder, generalized anxiety disorder, and phobias. Approximately 19.1 million American adults ages 18 to 54 (about 13.3% of people in this age group in a given year) have an anxiety disorder. In some embodiments, a patient is treated for an anxiety disorder. The anxiety disorder is one or more of panic disorder, obsessive-compulsive disorder, post-traumatic stress disorder, generalized anxiety disorder, or a phobia.

Further psychiatric conditions include schizophrenia. In a given year, over 2 million people are clinically diagnosed with schizophrenia, and there is a lifetime prevalence of this disease in approximately 1% of the U.S. population. Schizophrenia is a chronic, debilitating disease that leaves an estimated 75% of treated patients without ever achieving complete recovery. An exemplary schizophrenia is paranoid schizophrenia. Persons suffering paranoid schizophrenia are very suspicious of others and often have grand schemes of persecution at the root of their behavior. Hallucinations, and more frequently delusions, are a prominent and common part of the illness. Persons with disorganized schizophrenia (hebephrenic schizophrenia) are verbally incoherent and may have moods and emotions that are not appropriate to the situation. Hallucinations are not usually present with disorganized schizophrenia. Catatonic schizophrenia is where a person is extremely withdrawn, negative and isolated, and has marked psychomotor disturbances. Residual schizophrenia is where a person is not currently suffering from delusions, hallucinations, or disorganized speech and behavior, but lacks motivation and interest in day-to-day living. Schizoaffective disorder is where a person has symptoms of schizophrenia as well as mood disorder such as major depression, bipolar mania, or mixed mania. Undifferentiated schizophrenia is where conditions meet the general diagnostic criteria for schizophrenia but do not conform to any of the above subtypes, or there are features of more than one of the subtypes without a clear predominance of a particular set of diagnostic characteristics. In some embodiments, the patient is treated for schizophrenia.

Substance-related psychiatric conditions and disorders include a wide spectrum of distinct disorders, as known in the art. Exemplary substance-related disorders relate to alcohol, amphetamine, caffeine, cannibis, cocaine, hallucinogen, nicotine, opioid, phencyclidine, sedative, hyponetic and anxiolytic use. In some embodiments, the patient is treated for a substance-related psychiatric condition.

Sleep disorders include primary sleep disorders (e.g., primary hypersomnia, primary insomnia, nacrolepsy, breathing-related sleep disorder, circadian rhythm sleep disorder and dyssomnia), Parasomnias (e.g., nightmare disorder, sleep terror disorder, sleepwalking disorder, and parasomnia), and “other” sleep disorders due to a medical condition, as known in the art. In some embodiments, the patient is treated for a sleep disorder.

Exemplary somatoform disorders include somatization disorder characterized by chronic and persistent complaint of varied physical symptoms that have no identifiable physical origin, undifferentiated somatoform disorder, conversion disorder characterized by neurological symptoms such as numbness, paralysis, or fits, but where no neurological explanation can be found, pain disorder associated with psychological factor and/or a general medical condition, hypochrondriasis characterized by an excessive preoccupation or worry about having a serious illness, body dysmorphic order characterized by excessive concern about and preoccupation with a perceived defect in physical features, all known in the art. In some embodiments, the patient is treated for a somatoform disorder.

Another common psychiatric condition is eating disorders. There are three main types, anorexia nervosa, bulimia nervosa, and binge-eating disorders. These are psychiatric conditions are often linked to perceived notions about body image and are usually independent of actual body weight or body mass index. The mortality of people with anorexia has been estimated at 0.56 percent per year, or approximately 5.6 percent per decade, which is about 12 times higher than the annual death rate due to all causes of death among females ages 15-24 in the general population. See Sullivan, 1995, American Journal of Psychiatry 152: 1073-1074). As understood in the art, psychiatric illnesses usually present with elements of other psychiatric disorders. In some embodiments, the patient is treated for an eating disorder.

In some embodiments, there is provided a method of treating a mood disorder, an anxiety disorder or schizophrenia. In some embodiments, the disorder or disorder is an anxiety disorder, for example but limited to obsessive-compulsive disorder, as known in the art. In some embodiments, the disease or disorder is schizophrenia.

More particular types of the above named disorders can be found in the DSM-IV. The following are only examples of disorders that may be treated by the methods disclosed herein. Examples include mood disorders that may include depressive disorders and bipolar disorders. In some embodiments, the disease or disorder is depression. Mood disorders can further be characterized as major depressive disorders, dysthymic disorder, bipolar I disorder, bipolar II disorder, cyclothymic disorder, bipolar disorder not otherwise specified, mood disorders due to a medical condition, substance-induced mood disorder, or mood disorder not otherwise specified. Anxiety disorders can include panic disorder, specific phobia, social phobia, obsessive-compulsive disorder, posttraumatic stress disorder, acute stress disorder, generalized anxiety disorder, anxiety disorder due to a medical condition, substance induced anxiety disorder and anxiety disorder not otherwise specified.

In some embodiments, the disease or disorder is a substance-related disorder. Substance-related disorders include substance dependence, substance addiction, substance-induced anxiety disorder, and substance-induced mood disorder. Substance dependence and addiction can occur with a variety of substances, including but not limited to, alcohol, nicotine, cocaine, opioids, narcotics, hallucinogens, amphetamines, phencyclidines, phencyclidine-like substances, inhalants, and sedatives. Substance-induced anxiety disorder can occur in response to substances which include, but not limited to, caffeine, cannabis, cocaine, hallucinogens, amphetamines, phencyclidines, phencyclidine-like substances, and inhalants. Substance-induced mood disorder can occur in response to substances which include, but not limited to cocaine, hallucinogens, opioids, amphetamines, phencyclidines, phencyclidine-like substances, and inhalants. Substance-related disorders can occur in response to one substance or to a combination of substances, such as in polysubstance-related disorder.

In some embodiments, methods provided include the treatment of medication-induced psychiatric disorders or psychiatric disorders that result from treatment of a disease. For example, hedonistic homeostatic dysregulation is a neuropsychological behavioral disorder recognized in patients with Parkinson's disease undergoing dopamine replacement therapy. Dopamine replacement therapy in these patients appears to stimulate central dopaminergic pathways and lead to a behavioral disorder with some similarities to that associated with stimulant addiction. See e.g., Giovannoni et al., 2000, J. Neurol. Neurosurg. Psychiatry 68:423-428.

Eating disorders can include anorexia nervosa, bulimia nervosa, and eating disorders not otherwise specified. These eating disorders may include binge eating. In certain embodiments, methods provided are drawn to the treatment of the psychiatric illness associated with the eating disorder. In certain embodiments, methods provided may be used for treating the psychiatric illness associated with anorexia or binge eating.

In some embodiments, methods provided can be used to treat patients experiencing intermittent excessive behaviors (IEB). IEB characterize a variety of disorders including, binge eating, substance abuse, alcoholism, aberrant sexual conduct, and compulsive gambling. IEB occur when occasional normal behavioral excess is transformed into repetitive, intermittent, maladaptive behavioral excess. See, e.g., Corwin, 2006, Appetite 46: 11-15.

In certain embodiments, methods provided may not include the treatment of somatoform disorders. In certain embodiments, methods provided may include somatoform disorders but do not include the treatment of physical pain. In still other embodiments, methods provided may include the treatment of the psychiatric illness associated with pain.

In one general aspect, it is contemplated that compounds that reduce or moderate stress, or regulate the stress pathway, may be useful as pharmacotherapeutic agents. In another general aspect, it is contemplated that compounds that can affect or regulate metabolic disturbances as well as psychiatric or behavioral processes would be useful as pharmacotherapeutic agents. In another general aspect, it is contemplated that compounds that can attenuate or reverse metabolic disturbances would be useful as pharmacotherapeutic treatments of psychiatric diseases or disorders.

In another aspect, there is provided a method for the treatment in a patient in need of treatment for an eating disorder, insulin resistance, obesity, overweight, abnormal postprandial hyperglycemia, diabetes of any type including Type I, Type II and gestational diabetes, metabolic syndrome, dumping syndrome, hypertension, dyslipidemia, cardiovascular disease, hyperlipidemia, sleep apnea, cancer, pulmonary hypertension, cholescystitis and osteoarthritis. The method includes administering to a patient in need of treatment a compound or pharmaceutical composition described herein in an effective amount to treat the disease or disorder.

Obesity and its associated disorders including overweight are common and serious public health problems in the United States and throughout the world. Upper body obesity is the strongest risk factor known for type 2 diabetes mellitus and is a strong risk factor for cardiovascular disease. Obesity is a recognized risk factor for hypertension, atherosclerosis, congestive heart failure, stroke, gallbladder disease, osteoarthritis, sleep apnea, reproductive disorders such as polycystic ovarian syndrome, cancers of the breast, prostate, and colon, and increased incidence of complications of general anesthesia. See, e.g., Kopelman, 2000, Nature 404:635-43.

Methods for production and assay of compounds described herein are generally available to the skilled artisan. Representative assays for the compounds and methods described herein follow.

Food intake is useful in the assessment of the utility of a compound as described herein for use in the treatment of psychiatric indications. For example, it is known that a number of metabolic pathologies relating to food intake (e.g., diabetes, obesity) are associated with behavioral dysfunction. Accordingly, an initial screening can be conducted to determine the extent to which food intake is modulated by administration of compounds described herein, and a positive initial screening can be useful in subsequent development of a compound.

A variety of food intake assays are available to one of skill in the art. For example, in the so-called “home cage model” of food intake, patients (e.g., rats) are maintained in their home cage, and food intake along with total weight of the patient is measured following injection of test compound. In the so-called “feeding patterns model” of food intake assay, patients (e.g., rats) are habituated to a feeding chamber and to injections prior to testing. After test compound administration, the patients are immediately placed into the feeding chamber, and food intake is automatically determined as a function of time (e.g., 1-min intervals). For both tests, the food is standard chow or any of a variety of chows (e.g., high fat) known in the art. In the so-called “mouse food intake” assay, a test compound may be tested for appetite suppression, or for an effect on body weight gain in diet-induced obesity (DIO) mice. In a typical mouse food intake assay, female NIH/Swiss mice (8-24 weeks old) are group housed with a 12:12 hour light:dark cycle with lights on at 0600. Water and a standard pelleted mouse chow diet are available ad libitum, except as noted. Animals are fasted starting at approximately 1500 hrs, 1 day prior to experiment. The morning of the experiment, animals are divided into experimental groups. In a typical study, n=4 cages with 3 mice/cage. At time=0 min, all animals are given an intraperitoneal injection of vehicle or compound, typically in an amount ranging from about 10 nmol/kg to 75 nmol/kg, and immediately given a pre-weighed amount (10-15 g) of the standard chow. Food is removed and weighed at various times, typically 30, 60, and 120 minutes, to determine the amount of food consumed. See, e.g., Morley et al., 1994, Am. J. Physiol. 267:R178-R184). Food intake is calculated by subtracting the weight of the food remaining at the e.g. 30, 60, 120, 180 and/or 240 minute time point, from the weight of the food provided initially at time=0. Significant treatment effects are identified by ANOVA (p<0.05). Where a significant difference exists, test means are compared to the control mean using Dunnett's test (Prism v. 2.01, GraphPad Software Inc., San Diego, Calif.). For any test described herein, administration of test compound can be by any means, including injection (e.g., subcutaneous, intraperitoneal, and the like), oral, or other methods of administration known in the art.

Correlations exist between the results of in vitro (e.g., receptor) assays, and the utility of psychiatric agents for the treatment of such diseases and disorders. Accordingly, in vitro assays (e.g., cell based assays) are useful as a screening strategy for potential psychiatric agents, such as described herein. A variety of in vitro assays are known in the art, including those described as follows.

Calcitonin adenylate cyclase assay (Functional Assay). The calcitonin receptor mediated adenylate cyclase activation can be measured using an HTRF (Homogeneous Time-Resolved Fluorescence) cell-based cAMP assay kit from CisBio. This kit is a competitive immunoassay that uses cAMP labeled with the d2 acceptor fluorophore and an anti-cAMP monoclonal antibody labeled with donor Europium Cryptate. Increase in cAMP levels is registered as decrease in time-resolved fluorescence energy transfer between the donor and acceptor. Peptides can be serially diluted with buffer and transferred to, for example, a 384-well compound plate. C1a-HEK cells stably expressing the rat C1a calcitonin receptor can be detached from cell culture flasks and resuspended at 2×10⁶ cell/ml in stimulation buffer containing 500 μM IBMX, and d2 fluorophore at 1:40. Cells can be added to the compound plate at a density of 12,500 per well and incubated in the dark for 30 minutes at room temperature for receptor activation. Cells can be subsequently lysed by the addition of anti-cAMP Cryptate solution diluted with the kit conjugate/lysis buffer (1:40). After 1 to 24 hours incubation in the dark, the plate can be counted on a Tecan Ultra capable of measuring time-resolved fluorescence energy transfer.

Amylin receptor binding assay. RNA membranes can be incubated with approximately 20 μM (final concentration) of ¹²⁵I-rat amylin (Bolton-Hunter labeled, PerkinElmer, Waltham, Mass.) and increasing concentrations of test compound for 1 hour at ambient temperature in, for example, 96-well polystyrene plates. Bound fractions of well contents can be collected onto a 96 well glass fiber plate (pre-blocked for at least 30 minutes in 0.5% PEI (polyethyleneimine)) and washed with 1×PBS using a Perkin Elmer plate harvester. Dried glass fiber plates can be combined with scintillant and counted on a multi-well Perkin Elmer scintillation counter.

CGRP receptor binding assay. SK-N-MC cell membranes can be incubated with approximately 50 μM (final concentration) of ¹²⁵I-human CGRP (PerkinElmer, Waltham, Mass.) and increasing concentrations of test compound for 1 hour at ambient temperature in 96-well polystyrene plates. Bound fractions of well contents can be collected onto a 96 well glass fiber plate (pre-blocked for at least 30 minutes in 0.5% PEI) and washed with 1×PBS using a Perkin Elmer plate harvester. Dried glass fiber plates can be combined with scintillant and counted on a multiwell Perkin Elmer scintillation counter.

Calcitonin receptor binding assay. C1a-HEK cell membranes can be incubated with approximately 50 μM (final concentration) of ¹²⁵I-human calcitonin (PerkinElmer, Waltham, Mass.) and increasing concentrations of test compound for 1 hour at ambient temperature in, for example, 96-well polystyrene plates. Bound fractions of well contents can be collected onto a 96 well glass fiber plate (pre-blocked for at least 30 minutes in 0.5% PEI) and washed with 1×PBS using a Perkin Elmer plate harvester. Dried glass fiber plates can be combined with scintillant and counted on a multiwell Perkin Elmer scintillation counter.

Animal models of psychiatric disorders. Animal models of psychiatric disorders typically attempt to mimic a corresponding human psychopathology. Methods for assay of compounds described herein are generally available to the skilled artisan and include the following.

Stress-induced hyperthermia (SIH). Body temperature and emotional state are closely related in humans. Without wishing to be bound by any theory, it is believed that stress-induced hyperthermia (SIH) in rodents has predictive validity for certain human anxiety/stress disorders. The SIH assay assesses the effect of test agents (e.g, anxiolytics) on core body temperature following restraint stress. See, for example, Zethof et al., 1994, Physiol. Behay. 55:109-115. Anxiolytics typically blunt the increase in body temperature, or hyperthermic response, following stress exposure. Prior to being placed in a restrainer to induce the hyperthermic response, test animals can be administered test agents or control agents (e.g., vehicle, chlordiazepoxide and the like) at different pretreatment times (e.g., 1, 18, 24 or 36 hours, or even longer). Test animals can then be patiented to two sequential rectal temperature measurements at a measured time interval (e.g, 30 min). The difference between the second temperature reading and the first temperature reading (ΔT) is the stress-induced hyperthermic response.

Marble burying is used as a model for both anxiety and obsessive-compulsive disorder. See, for example, Chaki et al., 2003, J. Pharmacol. Exp. Ther. 304:818-826. Anxiolytics suppress marble burying activity. Without wishing to be bound by any theory, it is believed that marble burying is a useful pharmacological assay for detecting anxiolytics and SSRIs (selective serotonin reuptake inhibitors). In typical applications, mice can be injected with the agent or vehicle 15-30 minutes prior to the test. Mice can then be placed individually in clean cages containing hardwood bedding (e.g., 5-cm) and marbles (e.g., 20 marbles) spaced evenly (e.g., in rows of five). The number of marbles buried in 30 minutes can be recorded.

The forced swim test (FST) is a commonly used paradigm to evaluate antidepressant activity of drugs. The FST is based on measurement of the animal's floating time in a tank filled with water. When rats or mice are forced to swim in a deep cylinder with tepid water they become nearly immobile and cease trying to escape. Without wishing to be bound by any theory, it is believe that this characteristic immobile posture reflects a depressive-like state which is readily influenced by a wide variety of antidepressants. See, e.g., Hedou et al., 2001, Pharmacol, Biochem. Behay. 70:65-76; Chaki et al., 2003, J. Pharmacol. Exp. Ther. 304:818-826; Porsolt et al., 1977, Nature 266:730-732. Antidepressants decrease the immobility time in the FST. Vehicle or test compound can be delivered continuously for two weeks to mice by subcutaneously implanted osmotic pumps prior to the FST. Indeed, any route of administration (e.g., intraperitoneal, subcutaneous, oral and the like) is available. Mice can be placed in the water tank for assessment of climbing, swimming, and immobility over a defined trial session, typically 6 minutes. Test session parameters for mice and rats are typically different, as known in the art.

The prepulse inhibition (PPI) test measures the reflex response to externally applied auditory stimulation (acoustic startle response) and is believed to be related to the deficiency in sensory-motor gating capacity seen in schizophrenia. The acoustic startle reflex is a very basic response to strong exteroceptive stimuli and is widely used to assess sensorimotor reactivity in animals and humans. A weak auditory stimulus (prepulse, 74-82 dB) given prior to the strong acoustic stimulus (120 dB) blunts the startle response. This blunting of the startle response is referred to as prepulse inhibition. See, e.g., Conti et al., 2005, Behavioral Neuroscience 119:1052-1060. Antipsychotics increase the ability of the prepulse stimulus to blunt the startle response to the strong stimulus. Some psychotomimetic agents, such as phencyclidine (PCP) and ketamine, can actually reduce the percent prepulse inhibition and stimulate a psychotic-like state in animals, which can be antagonized by antipsychotic agents. Use of PCP in the PPI provides the so-called “PCP-PPI” model. In a typical application of the PPI test, mice can be injected with the test agent or vehicle 15 min prior to the test, or with haloperidol at 1 mg/kg 30 minutes prior to the test. The mice can be placed into an animal holder with the holder placed onto a transducer platform in an acoustic chamber. A weak (prepulse) auditory stimulus (e.g., 74, 78 and 82 dB) can be given prior to the strong acoustic stimulus (e.g., 120 dB). The reaction of the test animal to the strong stimulus can then be recorded. As known in the art, halperidol is a dopamine receptor antagonist and a first generation antipsychotic agent.

Phencyclidine (PCP)-induced locomotion (open field). The PCP-induced locomotion test is used with the open field activity chambers and measures locomotion, rearing, and stereotypic activity under amphetamine/PCP-induced conditions. The test has predictive validity for some antipsychotic drugs that normalize the hyperactivity and stereotypic behavior seen with amphetamine and PCP. See, e.g., Williams et al., 2006, Prog. Neuropsychopharmacol. Biol. Psychiatry 30:239-243. Mice can be injected with the test agent or vehicle 15-30 minutes prior injection with 5 mg/kg PCP. The animals can then be placed in the center of an open field, and activity can be recorded for 60 minutes. Administration of test compound and control (e.g., the antipsychotic positive control CZP) can reduce the total distance traveled across all types assessed (total, central, and peripheral) in the PCP-induced locomotion test.

EPM (Elevated Plus Maze). The elevated plus maze (EPM) is a rodent model of anxiety that is used as a screening test for putative anxiolytic compounds and as a general research tool in neurobiological anxiety research. The test setting consists of a plus-shaped apparatus with two open and two enclosed arms, each with an open roof, elevated 40-70 cm from the floor. The model is based on rodents' aversion of open spaces. This aversion leads to the behavior termed thigmotaxis, which involves avoidance of open areas by confining movements to enclosed spaces or to the edges of a bounded space. In EPM this translates into a restriction of movement to the enclosed arms. Anxiety reduction in the plus-maze is indicated by an increase in the proportion of time spent in the open arms (time in open arms/total time in open or closed arms), and an increase in the proportion of entries into the open arms (entries into open arms/total entries into open or closed arms). Total number of arm entries and number of closed-arm entries are usually employed as measures of general activity.

DOI-Head Shake. DOI (1-(2,5-dimethoxy-4-iodophenyl)-2-aminopropane hydrochloride) is a hallucinogen having high affinity and selectivity as an agonist at 5-HT2A/2C receptors. See, e.g., Dowd et al., 2000, J. Med. Chem., 43:3074-84; Yan Q S, 2000, Brain Res. Bull. 51:75-81; Wettstein et al., 1999, Prog. Neuropsychopharmacol. Biol. Psychiatry 23:533-44. In the DOI-induced head shake animal model, DOI administration produces dose-related behavioral effects including head shakes. In a dose-dependent manner, antipsychotics such as risperidone, haloperidol, clozapine and olanzapine antagonize the behavioral effects of DOI. Previous data show that antipsychotic agents as a drug class effectively block the effects of DOI with selective activity, and that non-antipsychotic drugs were generally inactive. See e.g., Wettstein et al., 1999, Prog. Neuropsychopharmacol. Biol. Psychiatry 23:533-44.

In the Conditioned Avoidance Response (CAR) test in the rat, test animals are trained to consistently avoid (by e.g., climbing onto a pole suspended from the ceiling of the test chamber) an electric foot shock (0.75 mA) delivered to the grid floor of the testing chamber, as known in the art. It has been found that antipsychotic drugs effectively inhibit this conditioned avoidance response. See, e.g., Arnt, 1982. Accordingly, the ability of a compound to inhibit this response is used to determine the antipsychotic efficacy of potential drug candidates.

The novel object recognition task, as known in the art, is widely used as a test of recognition memory. The test utilizes the natural tendency of rodents to explore a novel object rather than a familiar object when both are presented simultaneously. This test assesses the animal's ability to recall a familiar vis-a-vis novel object when re-exposed to the objects after a delay. The difference in time spent exploring each object during the test trial is used as an index of recognition of the previously explored, familiar object.

Novelty Induced Hypophagia assesses stress-induced anxiety by measuring the latency of an animal to approach and eat food in a novel environment. Acute injection of anxiolytics decreases latency to eat and increase food consumption in a novel environment.

The Morris water maze test assesses inter alia hippocampal-dependent spatial learning and memory. The test consists of a water pool with a hidden escape platform and using visual cues, rodents learn over the course of days to find the hidden platform and escape from the water.

In one aspect, there is provided a pharmaceutical composition which includes a peptide conjugate as described herein in combination with a pharmaceutically acceptable excipient.

The peptide conjugates described herein can be prepared and administered in a wide variety of oral, parenteral, and topical dosage forms. Thus, the peptide conjugates described herein can be administered by injection (e.g. intravenously, intramuscularly, intracutaneously, subcutaneously, intraduodenally, or intraperitoneally). Also, the peptide conjugates described herein can be administered by inhalation, for example, intranasally. Additionally, the peptide conjugates described herein can be administered transdermally. It is also envisioned that multiple routes of administration (e.g., intramuscular, oral, transdermal) can be used to administer the peptide conjugates described herein. Accordingly, the present invention also provides pharmaceutical compositions comprising a pharmaceutically acceptable carrier or excipient and one or more peptide conjugates described herein.

For preparing pharmaceutical compositions from the peptide conjugates described herein, pharmaceutically acceptable carriers can be either solid or liquid. Solid form preparations include powders, tablets, pills, capsules, cachets, suppositories, and dispersible granules. A solid carrier can be one or more substance that may also act as diluents, flavoring agents, binders, preservatives, tablet disintegrating agents, or an encapsulating material.

In powders, the carrier is a finely divided solid in a mixture with the finely divided active peptide. In tablets, the active peptide is mixed with the carrier having the necessary binding properties in suitable proportions and compacted in the shape and size desired.

The powders and tablets preferably contain from 5% to 70% of the active peptide conjugates described herein. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active peptide with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the peptide conjugates described herein are injectible, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The peptide conjugates described herein can also be incorporated into liposomes or administered via transdermal pumps or patches. Pharmaceutical admixtures suitable for use in the present invention include those described, for example, in PHARMACEUTICAL SCIENCES (17th Ed., Mack Pub. Co., Easton, Pa.) and WO 96/05309.

Aqueous solutions suitable for oral use can be prepared by dissolving the active peptide in water and adding suitable colorants, flavors, stabilizers, and thickening agents as desired. Aqueous suspensions suitable for oral use can be made by dispersing the finely divided active peptide in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active peptide, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active peptide. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active peptide in a unit dose preparation may be varied or adjusted from 0.001 mg to 1000 mg, from 0.01 mg to 500 mg, or from 0.1 mg to 10 mg, according to the particular application and the potency of the active peptide. The composition can, if desired, also contain other compatible therapeutic agents.

Some peptide conjugates described herein may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The compositions described herein may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760.

Pharmaceutical compositions described herein include compositions wherein peptide conjugates described herein is contained in a therapeutically effective amount, i.e., in an amount effective to achieve its intended purpose. The actual amount effective for a particular application will depend, inter alia, on the condition being treated. The dosage and frequency (single or multiple doses) of peptide conjugates described herein administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds of the invention.

For any peptide conjugates described herein, the therapeutically effective amount can be initially determined from a variety of assays, including but not limited to cell culture assays and behavioral assays. Target concentrations will be those concentrations of active compound(s) that are capable of eliciting a biological response in cell culture assay, or eliciting a behavioral response. Therapeutically effective amounts for use in humans may be determined from animal models. For example, a dose for humans can be formulated to achieve a concentration that has been found to be effective in animals. The dosage in humans can be adjusted by monitoring the underlying disease and adjusting the dosage upwards or downwards, as known in the art and/or as described herein.

### EXAMPLES

### Example 1

### Receptor Binding and Functional Activity

A variety of receptor binding and functional activity assays were conducted using the peptide conjugates described herein. Receptor binding activity can be expressed, for example in Table 6, as an IC₅₀ value, calculated from the raw data using an iterative curve-fitting program using a 4-parameter logistic equation (PRISM®, GraphPAD Software, La Jolla, Calif.), as known in the art.

For the amylin receptor binding assay, RNA membranes were incubated with approximately 20 μM (final concentration) of ¹²⁵I-rat amylin (Bolton-Hunter labeled, PerkinElmer, Waltham, Mass.) and increasing concentrations of test compound for 1 hour at ambient temperature in 96-well polystyrene plates. Bound fractions of well contents were collected onto a 96 well glass fiber plate (pre-blocked for at least 30 minutes in 0.5% PEI (polyethyleneimine)) and washed with 1×PBS using a Perkin Elmer plate harvester. Dried glass fiber plates were combined with scintillant and counted on a multi-well Perkin Elmer scintillation counter, as well known in the art.

For the calcitonin receptor binding assay, C1a-HEK cell membranes were incubated with approximately 50 μM (final concentration) of ¹²⁵I-human calcitonin (PerkinElmer, Waltham, Mass.) and increasing concentrations of test compound for 1 hour at ambient temperature in 96-well polystyrene plates. Bound fractions of well contents were collected onto a 96 well glass fiber plate (pre-blocked for at least 30 minutes in 0.5% PEI) and washed with 1×PBS using a Perkin Elmer plate harvester. Dried glass fiber plates were combined with scintillant and counted on a multiwell Perkin Elmer scintillation counter.

For the CGRP receptor binding assay, SK-N-MC cell membranes were incubated with approximately 50 pM (final concentration) of ¹²⁵I-human CGRP (PerkinElmer, Waltham, Mass.) and increasing concentrations of test compound for 1 hour at ambient temperature in 96-well polystyrene plates. Bound fractions of well contents were collected onto a 96 well glass fiber plate (pre-blocked for at least 30 minutes in 0.5% PEI) and washed with 1×PBS using a Perkin Elmer plate harvester. Dried glass fiber plates were combined with scintillant and counted on a multiwell Perkin Elmer scintillation counter.

For the calcitonin functional assay, the calcitonin receptor mediated adenylate cyclase activation was measured using an HTRF (Homogeneous Time-Resolved Fluorescence) cell-based cAMP assay kit from CisBio (Bedford, Mass.). This kit is a competitive immunoassay that uses cAMP labeled with the d2 acceptor fluorophore and an anti-cAMP monoclonal antibody labeled with donor Europium Cryptate. Increase in cAMP levels is registered as decrease in time-resolved fluorescence energy transfer between the donor and acceptor. Peptides were serially diluted with buffer and transferred to a 384-well plate. C1a-HEK cells stably expressing the rat C1a calcitonin receptor were detached from cell culture flasks and resuspended at 2×10⁶ cell/ml in stimulation buffer containing 500 μM IBMX, and d2 fluorophore at 1:40. Cells were added to the plate at a density of 12,500 per well and incubated in the dark for 30 minutes at room temperature for receptor activation. Cells were subsequently lysed by the addition of anti-cAMP Cryptate solution diluted with the kit conjugate/lysis buffer (1:40). After 1 to 24 hours incubation in the dark, the plate were counted on a Tecan Ultra capable of measuring time-resolved fluorescence energy transfer. In some assays, the data are normalized against the control peptide Cmpd 18 (EC₅₀=60 μM).

As shown in Table 7 below, the pegylated peptide can be generally less potent than the corresponding non-pegylated peptide (Cmpd 1) in binding and functional assays, although surprising deviations are observed. Specifically, removal of the N-terminal lysine of parent Cmpd 1 to provide Cmpd 2 and pegylation of the resulting peptide appears to reduce all binding or functional activity, with the exceptions that binding for Cmpd 169 is reduced less than 10-fold in the amylin and CGRP assays, and Cmpd 15 is surprisingly more potent in the calcitonin functional assay compared to either Cmpd 1 or Cmpd 2. It further appears that derivatization at any of positions of 11, 18 and 24 is highly detrimental to receptor binding and function.

TABLE 7
Receptor Binding and Functional Activity Assays

| | | | | | | | Calcitonin | |
|---|---|---|---|---|---|---|---|---|
| | | | | | | | Function | |
| | Receptor Binding, | | | | | | (Adenylate | |
| | IC₅₀ (nM) | | | | | | Cyclase), | |
| Cmpd | Amylin | | Calcitonin | | CGRP | | ED₅₀ (nM)¹ | |
| 1 | 0.044 ± 0.025 | (n = 20) | 0.092 ± 0.054 | (n = 11) | 4.5 ± 1.9 | (n = 15) | 0.073 ± 0.014 | (n = 4) |
| 2 | 0.16 ± 0.10 | (n = 2) | 0.21 ± 0.06 | (n = 2) | 3.8 ± 0.5 | (n = 2) | 0.029 ± 0.007 | (n = 2) |
| 15 | 0.042 | (n = 1) | nd | | 23 | (n = 1) | 0.042 | (n = 1) |
| 151 | 87 ± 15 | (n = 2) | 15 ± 4 | (n = 2) | >1000 | (n = 2) | 7.3 ± 3.5 | (n = 4) |
| 156 | 8.4 | (n = 1) | 5.2 | (n = 1) | >1000 | (n = 1) | 2.2 | (n = 1) |

| | | | | | | | |
|---|---|---|---|---|---|---|---|
| 169 | 17 ± 6 | (n = 2) | nd | >1000 | (n = 1) | 2.5 ± 1.6 | (n = 2) |

| | | | | | | |
|---|---|---|---|---|---|---|
| 170 | 15 | (n = 1) | nd | nd | 1.2 | (n = 1) |
| 171 | 11 ± 5 | (n = 2) | nd | nd | 6.5 ± 7.8 | (n = 2) |
| 172 | >100 | (n = 1) | nd | nd | >1000 | (n = 1) |
| 173 | >100 | (n = 1) | nd | nd | 19 | (n = 1) |
| 174 | >100 | (n = 1) | nd | nd | 130 | (n = 1) |

| | | | | |
|---|---|---|---|---|
| 179 | 295.26 | ND | ND | 8.09 |
| 180 | 7.422 | ND | ND | 1.08 |
| 181 | 13.44 | ND | ND | 3.66 |
| 182 | 37.633 | ND | ND | 3.75 |
| 183 | 21.244 | ND | ND | 1.88 |

¹Calcitonin functional assay (adenylate cyclase) data are normalized against Cmpd 18 (EC₅₀= 60 pM);
nd: not determined.

### Example 2

### Effect of mPEG on Food Intake and Body Weight

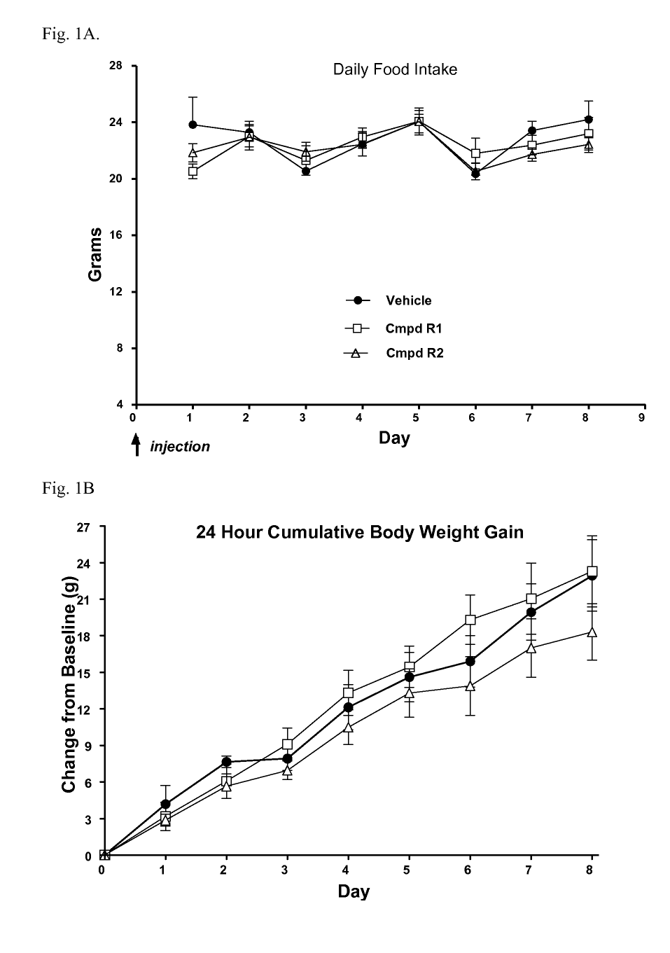

The effect of mPEG alone on 24-hr food intake and body weight gain was investigated for the chemically activated mPEG40 KD-NHS (Cmpd R1) and the corresponding chemically inert mPEG40 KD (Cmpd R2) compounds. Rats were administered a single subcutaneous (SC) injection of test compound or vehicle at the onset of the dark cycle. FIGS. 1A-B provides the result of a multi-day food intake assay which employed the home cage model, as described herein. The results of FIGS. 1A-B demonstrate that mPEG alone has no significant effect on food intake or body weight under the test conditions.

### Example 3

### Effect of Pegylation on Food Intake: Cmpds 151, 152, 153

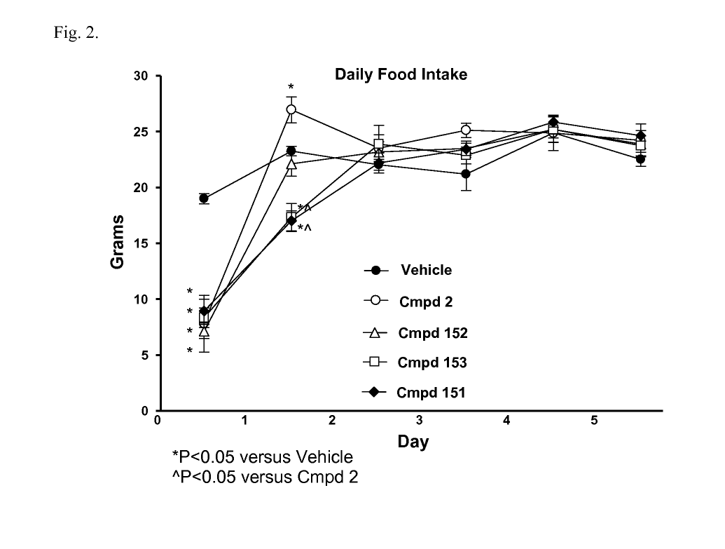

The effect on 24-hour food intake, as judged in the home cage model with intraperitoneal (IP) administration of test compound, was investigated for Cmpds 151, 152 and 153, using vehicle and Cmpd 2 as control. As depicted in FIG. 2, the presence of mPEG30 KD (Cmpd 153) and mPEG40 KD (Cmpd 151) at the N-terminal of peptide Cmpd 2 provides significantly enhanced duration of action with respect to 24-hours food intake compared with vehicle or peptide Cmpd 2 alone, with the effect lasting at least 36-hours post injection.

### Example 4

### Effect of Pegylation on Food Intake: Cmpds 160, 161, 162, 167

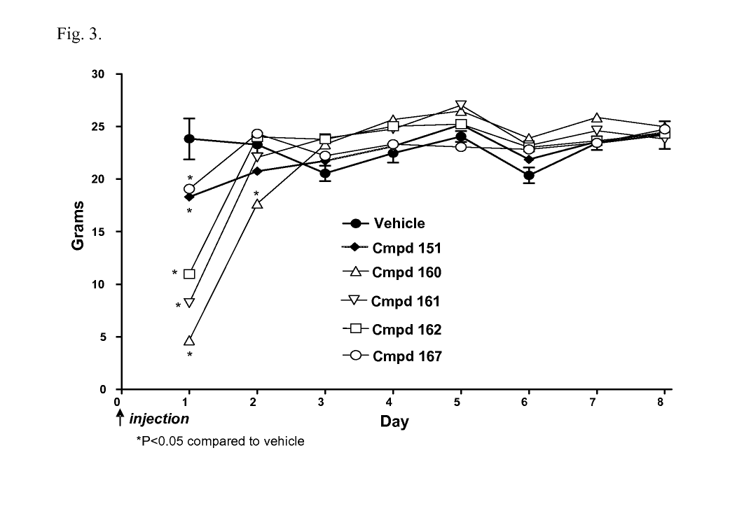

The effect on 24-hour food intake, as judged in the home cage food intake model with SC injection, of a single dose of a peptide having either a two-arm branched PEG (Cmpds 160, 161, 162) or the Warwick 40 KD PEG (Cmpd 167) at the N-terminal of the peptide was investigated. As shown in FIG. 3, a significant effect on food intake is observed under the experimental conditions for all peptides at the 24-hour mark, which reduction in food intake extends to at last 48-hours for the two-arm branched 40 KD pegylated Cmpd 160.

### Example 5

### Effect of Pegylation on Food Intake: Cmpds 151, 154, 155, 157

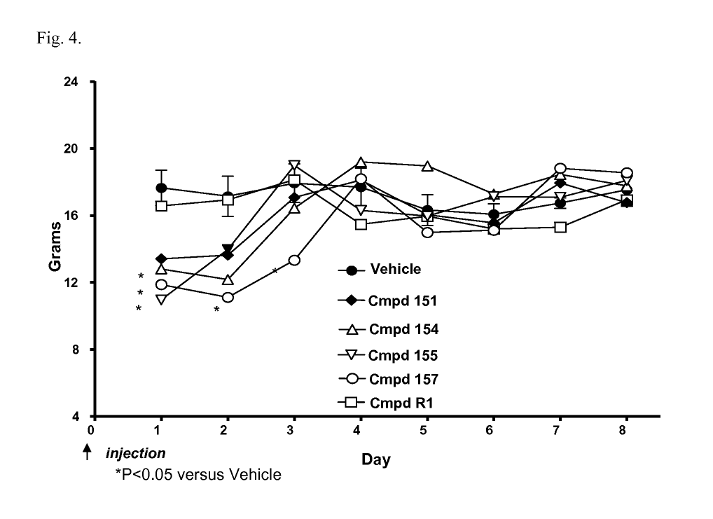

The effect on 24-hour food intake, as judged in the feeding pattern food intake model with SC injection, was investigated for Cmpds 151, 154, 155 and 157, and for the chemically activated PEG Cmpd R1. As shown in FIG. 4, each of the tested pegylated peptides demonstrate an effect on food intake for at least 48-hours post-injection in the feeding pattern assay.

### Example 6

### Effect of Pegylation on Food Intake: Cmpds 151, 156, 158, 159

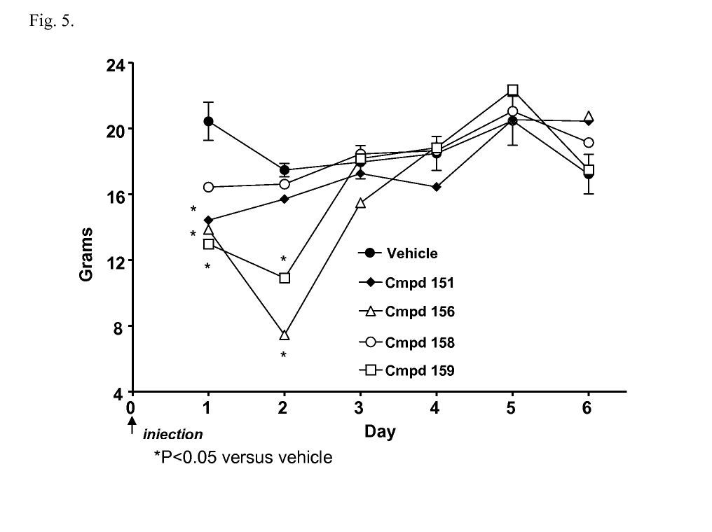

The effect on 24-hour food intake, as judged in the feeding pattern food intake model with SC injection, was investigated for Cmpds 151, 156, 158 and 159. As shown in FIG. 5, pegylation at residue 21 of the peptide provides significant effect on food intake under the experimental conditions.

### Example 7

### Effect of Pegylation on Food Intake: Cmpds 151, 156, 157, 169

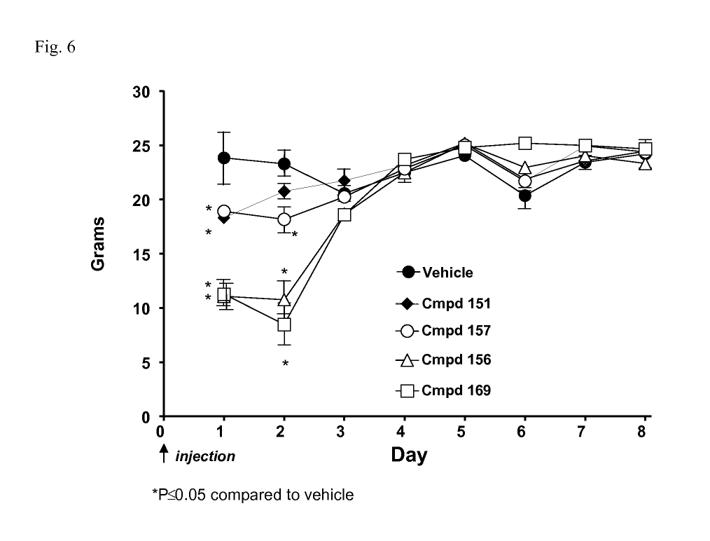

The effect on 24-hour food intake, as judged in the home cage food intake model with SC injection, was investigated for Cmpds 151, 156, 157 and 169. As shown in FIG. 6, pegylation at either of positions 21 or 26 of the peptide provides enhanced duration of action under the experimental conditions.

In summary, the food intake data set forth in Examples 3-7 provides valuable observations regarding the efficacy and effect on duration of action of pegylation of the peptide element of the tested compounds. Specifically, 30 KD and 40 KD PEG derivatives of peptide Cmpd 1 exhibit an extended time course of action compared to the non-pegylated peptide. The addition of a GGG linker increases the duration of action in the food intake assay, whereas attachment of the PEG at position 21 or 26 increased both duration of action and the magnitude of the food intake response. Two arm branched PEG peptides demonstrate greater efficacy on day 1 of the food intake assay compared to the linear PEG peptide. PEG alone, both chemically activated and chemically inert, has no effect on food intake or body weight.

### Example 8

### Initial Chronic Forced Swim Test of Pegylated and Non-Pegylated Peptides

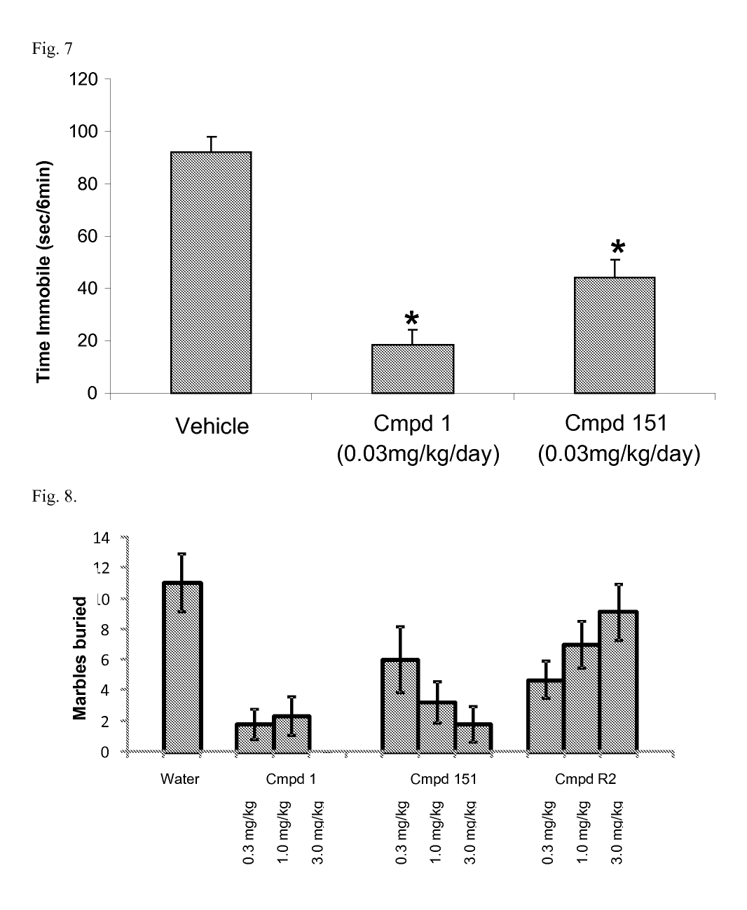

The forced swim test (FST) is a commonly used paradigm to evaluate antidepressant activity of drugs. An investigation of the effect on FST of pegylated Cmpd 151 with respect to the non-pegylated peptide Cmpd 1 was conducted. Specifically, vehicle or test compound was delivered continuously for two weeks to mice by subcutaneously implanted osmotic pumps prior to the FST assay, by methods well known in the art. On day one, the patients were introduced into the tank for a 15 minute pre-swim session. On day two, the patients were placed back into the water tank for assessment of climbing, swimming, and immobility over a 6 minute trial session. The rate of compound infusion was 0.03 mg/kg/day. As shown in FIG. 7, both the pegylated and non-pegylated peptides provide significant efficacy in reducing the immobilization time experienced by the patient animal.

### Example 9

### Marble Burying Assays

Marble burying is used as a model for both anxiety and obsessive-compulsive disorder. For the experiments described in FIG. 8, mice were injected with test compound (Cmpd 1, 151 or R1) or vehicle 30 minutes prior to the test. The administered amount of test compound was 0.3, 1.0 or 3.0 mg/kg, as indicated in FIG. 8. The mice were then placed individually in clean cages containing hardwood bedding and marbles (i.e., 20 marbles) spaced evenly in rows of five. The number of marbles buried in 30 minutes was recorded. As depicted in FIG. 8, both non-pegylated Cmpd 1 and pegylated Cmpd 151 were significantly effective in reducing marble burying.

### Example 10

### Stress Induced Hyperthermia: Description and Control Assays for PEG

As described above, it is believed that stress-induced hyperthermia (SIH) in rodents (e.g., rats) has predictive validity for certain human anxiety/stress disorders. Unless indicated differently, for the SIH experiments described herein, the following protocol was followed. Five days prior to the test, a programmable temperature device (IPTT-300) was implanted subcutaneously between the shoulder blades of male Harlan Sprague-Dawley rats (250 g) (n=5-7/group). Test animals were administered (IP injection) vehicle or test compound (10% saline) at t=−1080 (18 hr), −1440 minutes (24 hr) or −2160 minutes (36 hr) as indicated in the specific examples provided herein. At t=0 minutes, animals were placed in a physical restrainer (G3/G4 Braintree Scientific) for 30 minutes. Temperature readings were obtained remotely at the time of injection and at t=0 and t=30 minutes. The SIH response was defined as the change from t=30 back to t=0 minutes, while effects on basal temperature were calculated as the change from t=0 back to the time of injection.

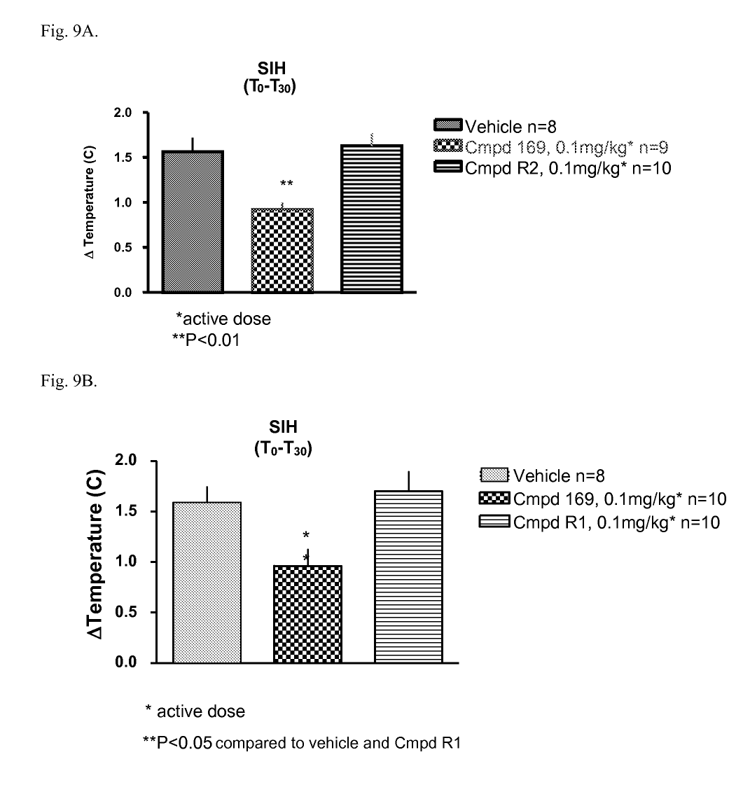

In order to assess the potential effect of mPEG on the SIH assay, Cmpd R2 (chemically inert mPEG40 KD) and Cmpd 169 (Acetyl-[desK¹, K²⁶(PEG40 KD)]-Cmpd 1) were administered with 18 hr pretreatment in the SIH assay described above using 0.1 mg/kg dosing. As shown in FIG. 9A, chemically inert mPEG alone has no effect on rat SIH with 18 hr pretreatment under these conditions. The corresponding experiment was conducted using chemically activated PEG Cmpd R1. As shown in FIG. 9B, chemically activated mPEG alone has no effect on rat SIH with 18 hr pretreatment under these conditions.

### Example 11

### Stress Induced Hyperthermia: Cmpd 169

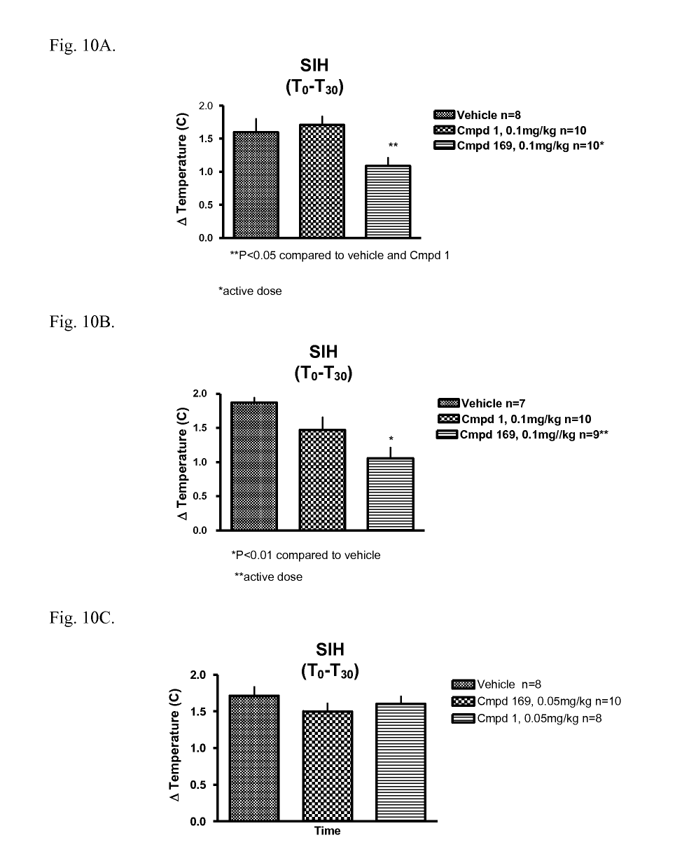

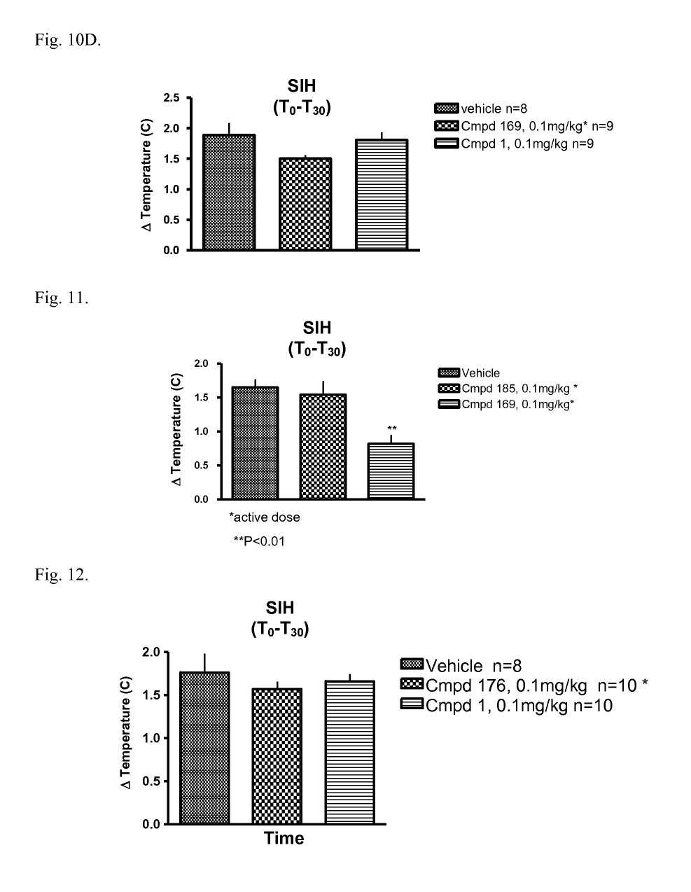

A comparison of pegylated and non-pegylated peptides in the SIH assay was conducted using Cmpd 1 or Cmpd 169 (Acetyl-[desK¹, K²⁶(PEG40 KD)]-Cmpd 1). As shown in FIG. 10A, at 0.1 mg/kg test compound and 18 hr pretreatment, differences in rat SIH are observed. As shown in FIG. 10B, at 0.1 mg/kg dosage and 24 hr pretreatment, an SIH effect is observed. As shown in FIG. 10C, lowering the dosage to 0.05 mg/kg with 18 hr pretreatment results in less change in hyperthermia; i.e., a dose dependence is observed. However, as shown in FIG. 10D, even administering 0.1 mg/kg Cmpd 169 or Cmpd 1 with 36 hr pretreatment demonstrates a statistically significant decrease in hyperthermia for the pegylated peptide.

### Example 12

### Stress Induced Hyperthermia: Cmpd 185

An SIH assay was conducted as described above to compare pegylated davalintide derivative Cmpd 185 with Cmpd 169. Peptides were administered at 0.1 mg/kg with an 18 hr pretreatment. As shown in FIG. 11, administration of Cmpd 169 resulted in less hyperthermia than observed with Cmpd 185.

### Example 13

### Stress Induced Hyperthermia: Cmpd 176

An SIH assay was conducted as described above to compare Cmpd 176 [K²¹(mPEG40 KD)]-Cmpd 2), pegylated at lysine 21 and missing the N-terminal lysine, with non-pegylated Cmpd 1. The dosing was 0.1 mg/kg, with an 18 hr pretreatment period, with results shown in FIG. 12.

### Example 14

### Stress Induced Hyperthermia: Cmpd 157

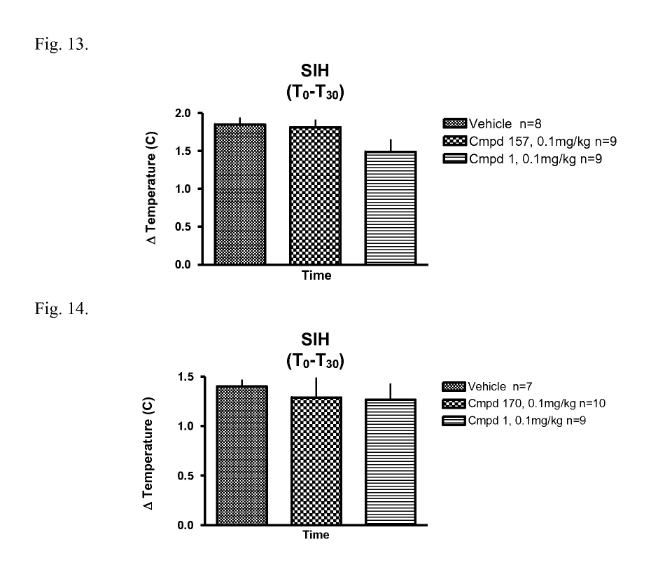

An SIH assay was conducted as described above to compare Cmpd 157, having a trisglycyl N-terminal linker to an mPEG40 KD moiety, and the non-pegylated Cmpd 1. The assay was conducted with 0.1 mg/kg dosing and 18 hr pretreatment, with results shown in FIG. 13.

### Example 15

### Stress Induced Hyperthermia: Cmpd 170

An SIH assay was conducted as described above to compare Cmpd 170, having a mPEG40 KD moiety at lysine 22, with the non-pegylated Cmpd 1. The assay was conducted with 0.1 mg/kg dosing and 18 hr pretreatment, with results shown in FIG. 14.

### Example 16

### Stress Induced Hyperthermia: Cmpd 156

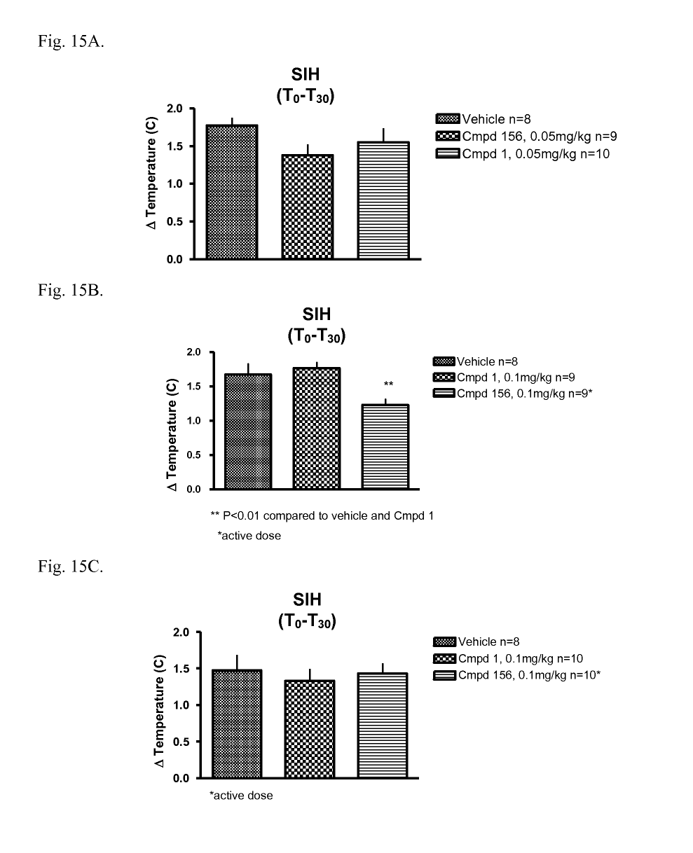

SIH assays were conducted as described above to compare Cmpd 156, having a mPEG40 KD moiety at lysine 21, with the non-pegylated Cmpd 1. As shown in FIG. 15A, an assay was conducted with 0.05 mg/kg dosing and 18 hr pretreatment and provided a reduction in hyperthermia for Cmpd 156. As shown in FIG. 15B, with 0.1 mg/kg dosing and 18 hr pretreatment, the hyperthermic effect of Cmpd 156 is more pronounced. As shown in FIG. 15C, increasing the pretreatment period to 24 hr with 0.1 mg/kg dosing results in a reduced hyperthermic effect for Cmpd 156.

### Example 17

### Stress Induced Hyperthermia: Cmpd 171

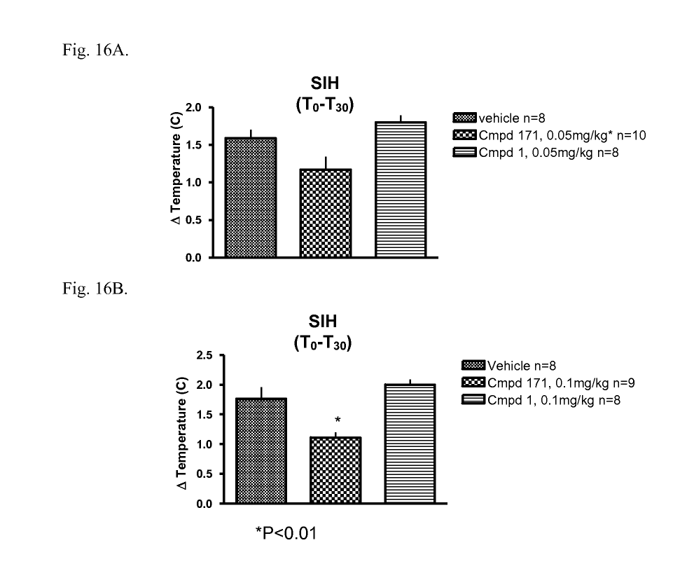

An SIH assay was conducted as described above to compare Cmpd 171, having a an mPEG40 KD moiety at lysine 23, with the non-pegylated Cmpd 1. As shown in FIG. 16A, one assay series was conducted with 0.05 mg/kg dosing and 18 hr pretreatment which resulted in a statistically significant reduction in hyperthermia for Cmpd 171. As shown in FIG. 16B, with 0.1 mg/kg dosing and 18 hr pretreatment, the hyperthermic effect of Cmpd 171 is also observed.

### Example 18

### Stress Induced Hyperthermia: Cmpd 151

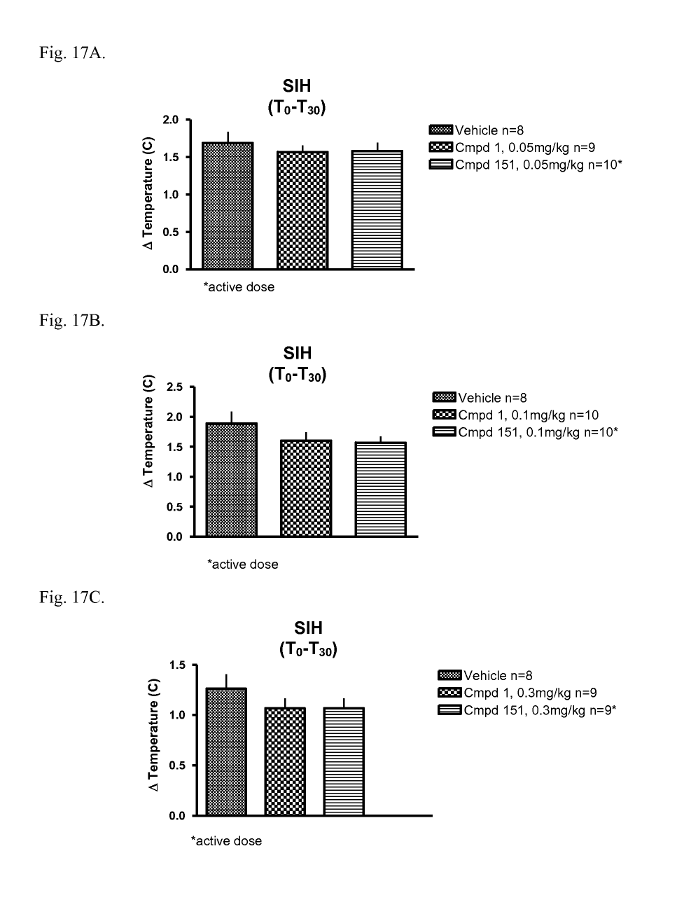

An SIH assay was conducted as described above to compare Cmpd 151, having a mPEG40 KD moiety at the N-terminal, with the non-pegylated Cmpd 1. As shown in FIG. 17A, one assay series was conducted with 0.05 mg/kg dosing and 18 hr pretreatment, which afforded a statistically insignificant SIH effect. As shown in FIG. 17B, with 0.1 mg/kg dosing and 18 hr pretreatment, the hyperthermic effect of Cmpd 151 is statistically significant with respect to vehicle. As shown in FIG. 17C, an increase in dosing to 0.3 mg/kg with 24 hr pretreatment results in a statistically significant decrease in hyperthermia for Cmpd 151.

### Example 19

### Stress Induced Hyperthermia: Cmpd 152

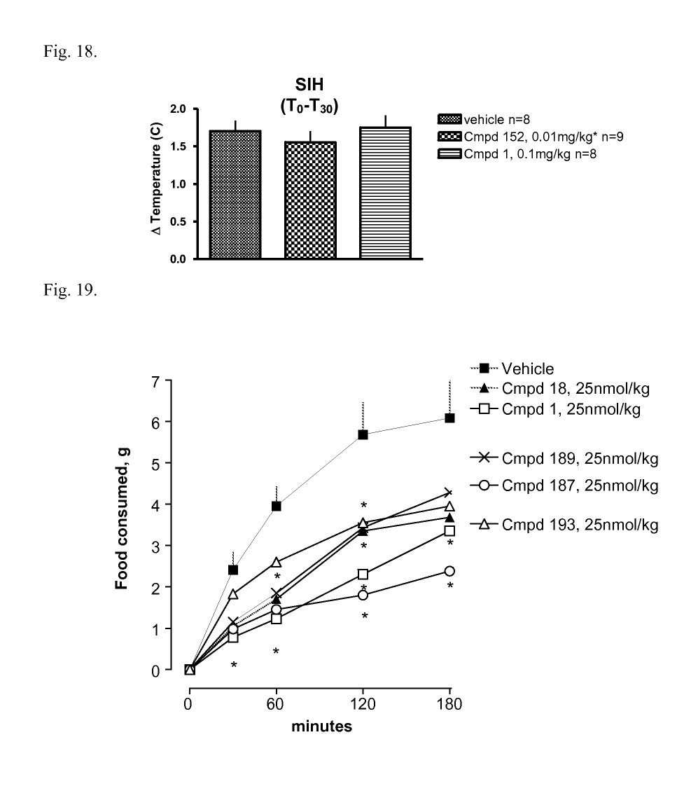

An SIH assay was conducted as described above to compare Cmpd 152, having a mPEG20 KD moiety at the N-terminal, with non-pegylated Cmpd 1. Dosing in this experiment was 0.01 mg/kg Cmpd 152 compared with 0.1 mg/kg Cmpd 1, with 18 hr pretreatment. As shown in FIG. 18, Cmpd 152 provides a statistically insignificant decrease in hyperthermia under the tested conditions.

### Example 20

### Receptor Binding: Fatty Acid Acylated Peptides

Receptor binding assays, as described in Example 1, were conducted on selected peptides having long chain fatty acid acylation, with results shown in Table 8 following. Also provided in Table 8 are the qualitative results from the FST assay conducted at 0.03 mg/kg dosing, as described in Example 8 above.

TABLE 8

| | Receptor Binding, | | | |
|---|---|---|---|---|
| | IC₅₀ (nM) | | | |
| Cmpd | Calcitonin | Amylin | CGRP | FST Assay, 0.03 mg/kg |
| 1 | 0.051 | 0.025 | 2.957 | |
| 17 | 0.095 | 0.028 | 1.262 | Inactive |
| 186 | 1.074 | 0.806 | 26.34 | Inactive |
| 192 | 0.07 | 0.518 | 4.45 | Active |
| 193 | 0.168 | 0.73 | 46.5 | Inactive |
| 187 | 0.151 | 0.446 | 0.77 | Inactive |
| 188 | 0.898 | 1.608 | 100 | Inactive |
| 189 | 1.487 | 4.014 | 75 | Active |
| 195 | 0.046 | 0.137 | 0.603 | Active |

### Example 21

### Mouse Food Intake Assay for Fatty Acid Acylated Peptides

A cumulative mouse food intake assay, as described herein, was conducted with fatty acid acylated Cmpds 189, 187 and 193, and with vehicle, Cmpd 1 and Cmpd 18 as control. With reference to FIG. 19, the points represent the mean+/−standard deviation (SD) for 3 mice/cage. Test compound was injected (IP) at t=0. Food was introduced immediately after injection, and the amount consumed was measured at T=30, 60, 120 and 180 min. The star (“*”) in the figure represents p<0.05 vs. vehicle control, calculated using ANOVA (Dunnett's test) as known in the art.

### Example 22

### Stress Induced Hyperthermia: Cmpd 189

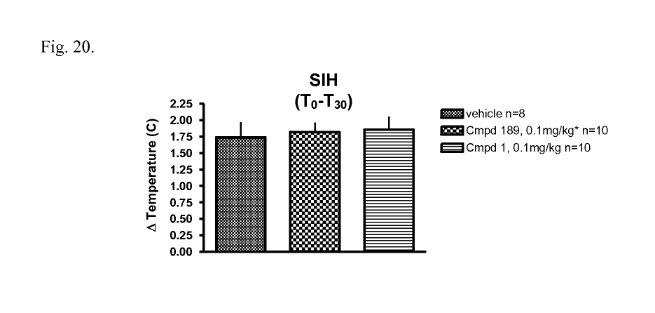

An SIH assay was conducted as described above to compare Cmpd 189, having a [K²⁶ε-(γ-Glu(N_{α}—C₁₆-Chain))] derivatization of Cmpd 17, with non-pegylated Cmpd 1. Experimental conditions were 0.1 mg/kg dosing, with an 18 hr pretreatment period. As shown in FIG. 20, Cmpd 189 provides statistically insignificant reduction in SIH under these experimental conditions.

### Example 23

### Plasma Concentration Assay

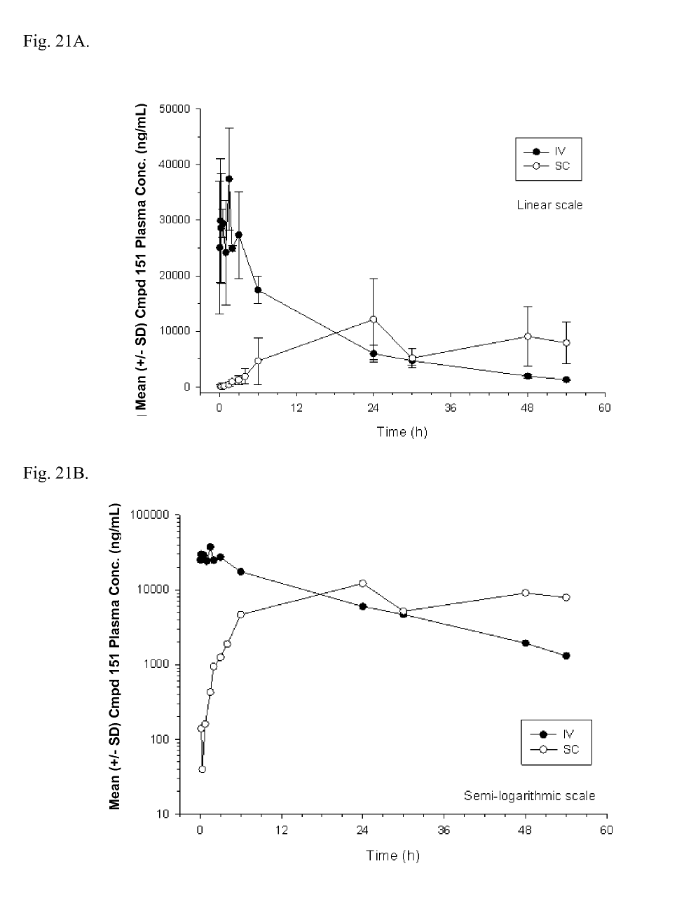

The pharmacokinetics (plasma concentration) of Cmpd 151 was determined over a 54 hr period for both IV and SC administration, using methods known in the art. As shown in FIG. 21A, IV administration provides an initial appearance of compound which decays with time in an approximate first-order reaction; see FIG. 21B, semi-logarithmic scale of the data provided in FIG. 21A. However, SC injection appears to provide an initial depot effect, such that after 24 hr, the plasma concentration via SC injection is greater than that observed for the corresponding IV injection.

### Example 24

### Stress Induced Hyperthermia: Cmpd 169, 171

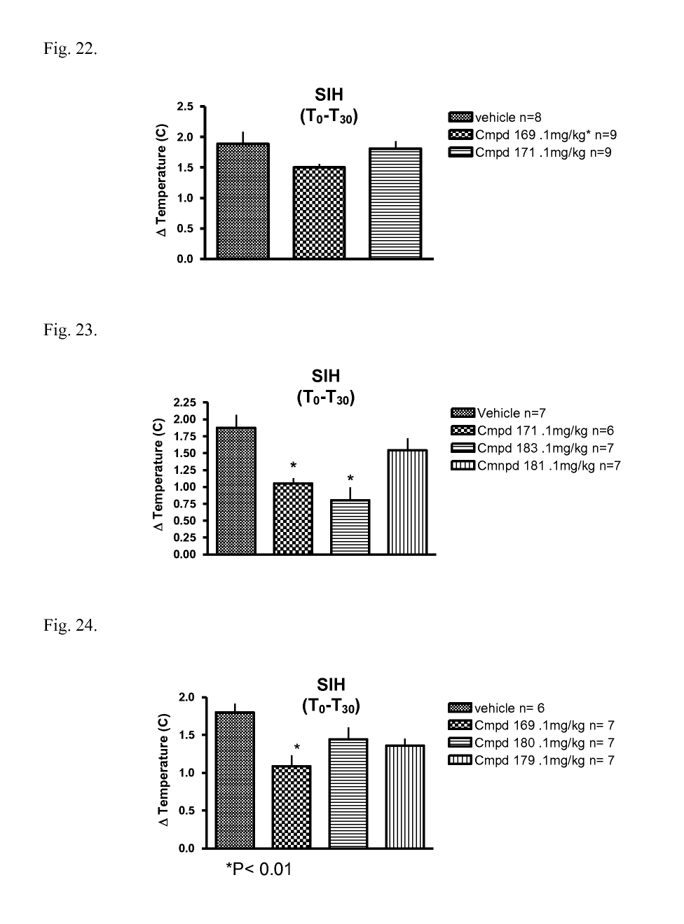

An SIH assay was conducted as described above to compare Cmpd 169, having a mPEG40 KD moiety at residue K²⁶, with Cmpd 171 having a mPEG40 KD moiety at residue K²³. Dosing in this experiment was 0.1 mg/kg for both Cmpd 169 and Cmpd 171, with 36 hr pretreatment. As shown in FIG. 22, both Cmpd 169 and Cmpd 171 provide statistically insignificant decreases in hyperthermia under the tested conditions.

### Example 25

### Stress Induced Hyperthermia: Cmpds 171, 183, 181

An SIH assay was conducted as described above to compare Cmpd 171, having a mPEG40 KD moiety at residue K²³, with Cmpd 183 having a NHCOO-mPEG40 KD moiety at residue K²⁶, and with Cmpd 181 having a two-arm branched mPEG40 KD at residue K²⁶. Dosing in this experiment was 0.1 mg/kg for all peptides, with 24 hr pretreatment. As shown in FIG. 23, Cmpds 171, 183 and 181 provide statistically significant decreases in hyperthermia under the tested conditions.

### Example 26

### Stress Induced Hyperthermia: Cmpds 169, 180, 179

An SIH assay was conducted as described above to compare Cmpd 169 with Cmpd 180 and Cmpd 179. Dosing in this experiment was 0.1 mg/kg for all peptides, with 24 hr pretreatment. As shown in FIG. 24, Cmpds 169, 180 and 179 each provide statistically significant decreases in hyperthermia under the tested conditions.

### Example 27

### Stress Induced Hyperthermia: Cmpds 169, 182

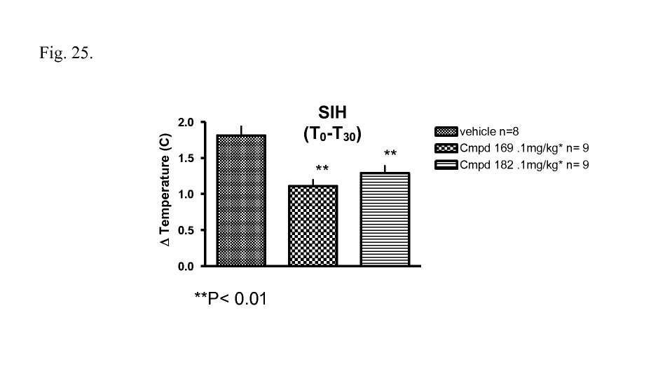

An SIH assay was conducted as described above to compare Cmpd 182 with Cmpd 169. Dosing in this experiment was 0.1 mg/kg for all peptides, with 24 hr pretreatment. As shown in FIG. 25, Cmpds 169 and 182 each provide statistically significant decreases in hyperthermia under the tested conditions.

### I. Embodiments

### Embodiment 1

A peptide conjugate comprising a peptide covalently linked to a duration enhancing moiety, wherein the peptide includes an amino acid sequence of residues 1-32 of Formula (I):

| | |
|---|---|
| X′-Xaa¹-Cys²-Asn³-Thr⁴-Ala⁵-Thr⁶-Cys⁷-Val⁸-Leu⁹- | (I) |
| Gly¹⁰-Arg¹¹-Leu¹²-Ser¹³-Gln¹⁴-Glu¹⁵-Leu¹⁶-His¹⁷-Arg¹⁸- | |
| Leu¹⁹-Gln²⁰-Thr²¹-Tyr²²-Pro²³-Arg²⁴-Thr²⁵-Asn²⁶-Xaa²⁷- | |
| Gly²⁸-Ser²⁹-Asn³⁰-Thr³¹-Xaa³²-X | |

wherein up to 25% of the amino acids set forth in Formula (I) may be deleted or substituted with a different amino acid; wherein X′ is hydrogen, an N-terminal capping group, a bond to a duration enhancing moiety, or a linker to a duration enhancing moiety; Xaa¹ is Lys or a bond; Xaa²⁷ is Thr or Val; Xaa³² is Tyr or a bond; and X is substituted or unsubstituted amino, substituted or unsubstituted alkylamino, substituted or unsubstituted dialkylamino, substituted or unsubstituted cycloalkylamino, substituted or unsubstituted arylamino, substituted or unsubstituted aralkylamino, substituted or unsubstituted alkyloxy, substituted or unsubstituted aryloxy, substituted or unsubstituted aralkyloxy, hydroxyl, a bond to a duration enhancing moiety, or a linker to a duration enhancing moiety; wherein the duration enhancing moiety is covalently linked, optionally through a linker, to a side chain of a linking amino acid residue, X′ or X.

### Embodiment 2

The peptide conjugate according to embodiment 1, wherein the duration enhancing moiety is a polyethylene glycol, a long chain acyl fatty acid or a derivative thereof.

### Embodiment 3

The peptide conjugate according to any of embodiments 1-2, wherein the linking amino acid residue is cysteine or lysine.

### Embodiment 4

The peptide conjugate according to any of embodiments 1-3, wherein the duration enhancing moiety is polyethylene glycol or derivative thereof.

### Embodiment 5

The peptide conjugate according to embodiment 4, wherein the polyethylene glycol is linear, branched or comb type.

### Embodiment 6

The peptide conjugate according to any of embodiments 1-5, wherein the peptide conjugate comprises only one the duration enhancing moiety.

### Embodiment 7

The peptide conjugate according to any of embodiments 1-6, wherein the duration enhancing moiety is attached to the N-terminal amino acid residue of the peptide.

### Embodiment 8

The peptide conjugate according to any of embodiments 1-6, wherein the duration enhancing moiety is attached to the C-terminal amino acid residue of the peptide.

### Embodiment 9

The peptide conjugate according to any of embodiments 1-6, wherein the duration enhancing moiety is attached to the side chain of the amino acid at position 11, 18, 21, 22, 23, 24 or 26.

### Embodiment 10

The peptide conjugate according to any of embodiments 1-3, wherein the duration enhancing moiety is a long chain fatty acid.

### Embodiment 11

The peptide conjugate according to embodiment 10, wherein the long chain fatty acid is C₆-C₂₄, C₈-C₂₀, C₁₀-C₁₈, or C₁₂-C₁₆.

### Embodiment 12

A pharmaceutical composition comprising a peptide conjugate according to any one of embodiments 1-11, and a pharmaceutically acceptable excipient.

### Embodiment 13

A method for treating a psychiatric disease or disorder in a patient comprising administering according to any of embodiments 1-12 to a patient in need of treatment in an amount effective to treat the disease or disorder.

### Embodiment 14

The method according to embodiment 13, wherein the disease or disorder is a mood disorder, an anxiety disorder or schizophrenia.

### Embodiment 15

The method according to embodiment 14, wherein the mood disorder is depression.

### Embodiment 16

The method according to embodiment 13, wherein the disease or disorder is an eating disorder, insulin resistance, obesity, overweight, abnormal postprandial hyperglycemia, Type I diabetes, Type II diabetes, gestational diabetes, metabolic syndrome, dumping syndrome, hypertension, dyslipidemia, cardiovascular disease, hyperlipidemia, sleep apnea, cancer, pulmonary hypertension, cholescystitis or osteoarthritis.

1. A peptide conjugate comprising a peptide covalently linked to a duration enhancing moiety, wherein said peptide comprises an amino acid sequence of residues 1-32 of Formula (I):

| | |
|---|---|
| X′-Xaa¹-Cys²-Asn³-Thr⁴-Ala⁵-Thr⁶-Cys⁷-Val⁸-Leu⁹- | (I) |
| Gly¹⁰-Arg¹¹-Leu¹²-Ser¹³-Gln¹⁴-Glu¹⁵-Leu¹⁶-His¹⁷-Arg¹⁸- | |
| Leu¹⁹-Gln²⁰-Thr²¹-Tyr²²-Pro²³-Arg²⁴-Thr²⁵-Asn²⁶-Xaa²⁷- | |
| Gly²⁸-Ser²⁹-Asn³⁰-Thr³¹-Xaa³²-X | |

wherein up to 25% of the amino acids set forth in Formula (I) may be deleted or substituted with a different amino acid;
wherein
X′ is hydrogen, an N-terminal capping group, a bond to a duration enhancing moiety, or a linker to a duration enhancing moiety;
Xaa¹ is Lys or a bond;
Xaa²⁷ is Thr or Val;
Xaa³² is Tyr or a bond; and
X is substituted or unsubstituted amino, substituted or unsubstituted alkylamino, substituted or unsubstituted dialkylamino, substituted or unsubstituted cycloalkylamino, substituted or unsubstituted arylamino, substituted or unsubstituted aralkylamino, substituted or unsubstituted alkyloxy, substituted or unsubstituted aryloxy, substituted or unsubstituted aralkyloxy, hydroxyl, a bond to a duration enhancing moiety, or a linker to a duration enhancing moiety;
wherein said duration enhancing moiety is covalently linked, optionally through a linker, to a side chain of a linking amino acid residue, X′ or X.

2. The peptide conjugate according to claim 1, wherein said duration enhancing moiety is a polyethylene glycol, a long chain acyl fatty acid or a derivative thereof.

3. The peptide conjugate according to claim 1, wherein said linking amino acid residue is cysteine or lysine.

4. The peptide conjugate according to claim 1, wherein said duration enhancing moiety is polyethylene glycol or derivative thereof.

5. The peptide conjugate according to claim 4, wherein said polyethylene glycol is linear, branched or comb type.

6. The peptide conjugate according to claim 1, wherein said peptide conjugate comprises only one said duration enhancing moiety.

7. The peptide conjugate according to claim 1, wherein said duration enhancing moiety is attached to the N-terminal amino acid residue of said peptide.

8. The peptide conjugate according to claim 1, wherein said duration enhancing moiety is attached to the C-terminal amino acid residue of said peptide.

9. The peptide conjugate according to claim 1, wherein said duration enhancing moiety is attached to the side chain of the amino acid at position 11, 18, 21, 22, 23, 24 or 26.

10. The peptide conjugate according to claim 1, wherein said duration enhancing moiety is a long chain fatty acid.

11. The peptide conjugate according to claim 10, wherein said long chain fatty acid is C₆-C₂₄, C₈-C₂₀, C₁₀-C₁₈, or C₁₂-C₁₆.

12. A pharmaceutical composition comprising a peptide conjugate according to claim 1, and a pharmaceutically acceptable excipient.

13. A method for treating a psychiatric disease or disorder in a patient comprising administering according to claim 1 to a patient in need of treatment in an amount effective to treat the disease or disorder.

14. The method according to claim 13, wherein said disease or disorder is a mood disorder, an anxiety disorder or schizophrenia.

15. The method according to claim 14, wherein said mood disorder is depression.

16. The method according to claim 13, wherein said disease or disorder is an eating disorder, insulin resistance, obesity, overweight, abnormal postprandial hyperglycemia, Type I diabetes, Type II diabetes, gestational diabetes, metabolic syndrome, dumping syndrome, hypertension, dyslipidemia, cardiovascular disease, hyperlipidemia, sleep apnea, cancer, pulmonary hypertension, cholescystitis or osteoarthritis.


SEQUENCE LISTING (214 sequences)

SEQ ID NO: 1 - protein, 37 aa, Rattus sp.
KCNTATCATQRLANFLVRSSNNLGPVLPPTNVGSNTY

SEQ ID NO: 2 - protein, 37 aa, Homo sapiens [sequence omitted]

SEQ ID NO: 3 - protein, 37 aa, Artificial Sequence
  Description of Artificial Sequence Synthetic polypeptide
KCNTATCATQRLANFLVHSSNNFGPILPPTNVGSNTY

SEQ ID NO: 4 - protein, 32 aa, Homo sapiens
CGNLSTCMLGTYTQDFNKFHTFPQTAIGVGAP

SEQ ID NO: 5 - protein, 32 aa, Unknown
  Description of Unknown Salmon calcitonin peptide
CSNLSTCVLGKLSQELHKLQTYPRTNTGSGTP

SEQ ID NO: 6 - protein, 32 aa, Artificial Sequence
  Description of Artificial Sequence Synthetic polypeptide
KCNTATCVLGRLSQELHRLQTYPRTNTGSNTY

SEQ ID NO: 7 - protein, 32 aa, Artificial Sequence
  Description of Artificial Sequence Synthetic polypeptide
KCNTATCVLGRLSQELHRLQTYPRTNXGSNTY

SEQ ID NO: 8 - protein, 32 aa, Artificial Sequence
  Description of Artificial Sequence Synthetic polypeptide
KXNTATXVLGRLSQELHRLQTYPRTNXGSNTY

SEQ ID NO: 9 - protein, 4 aa, Artificial Sequence
  Description of Artificial Sequence Synthetic peptide
GGGG

SEQ ID NO: 10 - protein, 5 aa, Artificial Sequence
  Description of Artificial Sequence Synthetic peptide
GGGGG

SEQ ID NO: 11 - protein, 8 aa, Artificial Sequence
  Description of Artificial Sequence Synthetic peptide
GGGKGGGG

SEQ ID NO: 12 - protein, 8 aa, Artificial Sequence
  Description of Artificial Sequence Synthetic peptide
GGGNGSGG

SEQ ID NO: 13 - protein, 8 aa, Artificial Sequence
  Description of Artificial Sequence Synthetic peptide
GGGCGGGG

SEQ ID NO: 14 - protein, 5 aa, Artificial Sequence
  Description of Artificial Sequence Synthetic peptide
GPNGG

SEQ ID NO: 15 - protein, 12 aa, Artificial Sequence
  Description of Artificial Sequence Synthetic peptide
GSGSGSGSGSGS

SEQ ID NO: 16 - protein, 18 aa, Artificial Sequence
  Description of Artificial Sequence Synthetic peptide
GGSGGSGGSGGSGGSGGS

SEQ ID NO: 17 - protein, 24 aa, Artificial Sequence
  Description of Artificial Sequence Synthetic peptide
GGGSGGGSGGGSGGGSGGGSGGGS

SEQ ID NO: 18 - protein, 30 aa, Artificial Sequence
  Description of Artificial Sequence Synthetic polypeptide
GGGGSGGGGSGGGGSGGGGSGGGGSGGGGS

SEQ ID NO: 19 - protein, 15 aa, Artificial Sequence
  Description of Artificial Sequence Synthetic peptide
GGGGSGGGGSGGGGS

SEQ ID NO: 20 - protein, 32 aa, Artificial Sequence
  Description of Artificial Sequence Synthetic polypeptide
KCNTATCVLGRLSQELHRLQTYPRTNVGSNTY

SEQ ID NO: 21 - protein, 31 aa, Artificial Sequence
  Description of Artificial Sequence Synthetic polypeptide
CNTATCVLGRLSQELHRLQTYPRTNVGSNTY

SEQ ID NO: 22 - protein, 32 aa, Artificial Sequence
  Description of Artificial Sequence Synthetic polypeptide
KCNTATCVLGRLSQELHRLQKYPRTNVGSNTY

SEQ ID NO: 23 - protein, 31 aa, Artificial Sequence
  Description of Artificial Sequence Synthetic polypeptide
CNTATCVLGKLSQELHRLQTYPRTNVGSNTY

SEQ ID NO: 24 - protein, 31 aa, Artificial Sequence
  Description of Artificial Sequence Synthetic polypeptide
CNTATCVLGRLSQELHKLQTYPRTNVGSNTY

SEQ ID NO: 25 - protein, 31 aa, Artificial Sequence
  Description of Artificial Sequence Synthetic polypeptide
CNTATCVLGRLSQELHRLQTKPRTNVGSNTY

SEQ ID NO: 26 - protein, 31 aa, Artificial Sequence
  Description of Artificial Sequence Synthetic polypeptide
CNTATCVLGRLSQELHRLQTYKRTNVGSNTY

SEQ ID NO: 27 - protein, 31 aa, Artificial Sequence
  Description of Artificial Sequence Synthetic polypeptide
CNTATCVLGRLSQELHRLQTYPKTNVGSNTY

SEQ ID NO: 28 - protein, 32 aa, Artificial Sequence
  Description of Artificial Sequence Synthetic polypeptide
CNTATCVLGRLSQELHRLQTYPRTNVGSNTYK

SEQ ID NO: 29 - protein, 34 aa, Artificial Sequence
  Description of Artificial Sequence Synthetic polypeptide
GGGCNTATCVLGRLSQELHRLQTYPRTNVGSNTY

SEQ ID NO: 30 - protein, 34 aa, Artificial Sequence
  Description of Artificial Sequence Synthetic polypeptide
CNTATCVLGRLSQELHRLQKGGGYPRTNVGSNTY

SEQ ID NO: 31 - protein, 31 aa, Artificial Sequence
  Description of Artificial Sequence Synthetic polypeptide
CNTATCVLGRLSQELHRLQKYPRTNVGSNTY

SEQ ID NO: 32 - protein, 32 aa, Artificial Sequence
  Description of Artificial Sequence Synthetic polypeptide
KCNTATCVLGRLADFLHRFHTFPRTNTGSNTY

SEQ ID NO: 33 - protein, 31 aa, Artificial Sequence
  Description of Artificial Sequence Synthetic polypeptide
CNTATCVLGRLADFLHRFHTFPRTNTGSNTY

SEQ ID NO: 34 - protein, 31 aa, Artificial Sequence
  Description of Artificial Sequence Synthetic polypeptide
CNTATCVLGRLSQELHRLQTYPRTKVGSNTY

SEQ ID NO: 35 - protein, 32 aa, Artificial Sequence
  Description of Artificial Sequence Synthetic polypeptide
SCNTATCVLGRLADFLHRLQTYPRTNTGSNTY

SEQ ID NO: 36 - protein, 32 aa, Artificial Sequence
  Description of Artificial Sequence Synthetic polypeptide
KCNTATCALQRLAQELHRLQTYPRTNVGSNTY

SEQ ID NO: 37 - protein, 32 aa, Artificial Sequence; identical to SEQ ID NO: 6
  Description of Artificial Sequence Synthetic polypeptide

SEQ ID NO: 38 - protein, 31 aa, Artificial Sequence; identical to SEQ ID NO: 31
  Description of Artificial Sequence Synthetic polypeptide

SEQ ID NO: 39 - protein, 32 aa, Artificial Sequence; identical to SEQ ID NO: 22
  Description of Artificial Sequence Synthetic polypeptide

SEQ ID NO: 40 - protein, 32 aa, Artificial Sequence
  Description of Artificial Sequence Synthetic polypeptide
KCNTATCVLGRLSQELHRLQTYPRTKVGSNTY

SEQ ID NO: 41 - protein, 32 aa, Artificial Sequence; identical to SEQ ID NO: 5
  Description of Artificial Sequence Synthetic polypeptide

SEQ ID NO: 42 - protein, 32 aa, Artificial Sequence; identical to SEQ ID NO: 4
  Description of Artificial Sequence Synthetic polypeptide

SEQ ID NO: 43 - protein, 31 aa, Artificial Sequence
  Description of Artificial Sequence Synthetic polypeptide
KCNTATCVLGRLSQELHRLQTYPRTNVSEAF

SEQ ID NO: 44 - protein, 32 aa, Artificial Sequence
  Description of Artificial Sequence Synthetic polypeptide
KCNTATCVLGRLTEFLHRLQTYPRTNTGSNTY

SEQ ID NO: 45 - protein, 32 aa, Artificial Sequence
  Description of Artificial Sequence Synthetic polypeptide
KCNTATCVLGRLAAALHRLQTYPRTNTGSNTY

SEQ ID NO: 46 - protein, 32 aa, Artificial Sequence
  Description of Artificial Sequence Synthetic polypeptide
KCNTATCVLGRLNDLLHRLQTYPRTNTGSNTY

SEQ ID NO: 47 - protein, 32 aa, Artificial Sequence
  Description of Artificial Sequence Synthetic polypeptide
KCNTATCVLGRLAAFLHRLQTYPRTNTGSNTY

SEQ ID NO: 48 - protein, 32 aa, Artificial Sequence
  Description of Artificial Sequence Synthetic polypeptide
KCNTATCATQRLANELVRLQTYPRTNVGSNTY

SEQ ID NO: 49 - protein, 32 aa, Artificial Sequence
  Description of Artificial Sequence Synthetic polypeptide
KCNTATCVLGRLYDYLHRLQTYPRTNTGSNTY

SEQ ID NO: 50 - protein, 32 aa, Artificial Sequence
  Description of Artificial Sequence Synthetic polypeptide
KCNTATCVLGRLFDFLHRLQTYPRTNTGSNTY

SEQ ID NO: 51 - protein, 32 aa, Artificial Sequence
  Description of Artificial Sequence Synthetic polypeptide
KCNTATCVLGRLSQELHXLQTYPRTNTGSNTY

SEQ ID NO: 52 - protein, 32 aa, Artificial Sequence
  Description of Artificial Sequence Synthetic polypeptide
KDNTATKVLGRLSQELHRLQTYPRTNTGSNTY

SEQ ID NO: 53 - protein, 32 aa, Artificial Sequence
  Description of Artificial Sequence Synthetic polypeptide
KCDTATCVTHRLSQELHRLQTYPRTNTGSNTY

SEQ ID NO: 54 - protein, 32 aa, Artificial Sequence
  Description of Artificial Sequence Synthetic polypeptide
KCNTATCVLGRLADFLHRFQTFPRTNTGSGTP

SEQ ID NO: 55 - protein, 32 aa, Artificial Sequence
  Description of Artificial Sequence Synthetic polypeptide
SCNTATCVLGRLSQELHRLQTYPRTNTGSNTY

SEQ ID NO: 56 - protein, 32 aa, Artificial Sequence
  Description of Artificial Sequence Synthetic polypeptide
KCNTATCVLGRLSQELHRLQTYPRTNTGSKAF

SEQ ID NO: 57 - protein, 34 aa, Artificial Sequence
  Description of Artificial Sequence Synthetic polypeptide
GCNTATCQVQNLSHRLWQLRQDSAPVDPSSPHSY

SEQ ID NO: 58 - protein, 32 aa, Artificial Sequence
  Description of Artificial Sequence Synthetic polypeptide
CSNLSTCVLGRLSQELHRLQTYPRTNTGSNTY

SEQ ID NO: 59 - protein, 32 aa, Artificial Sequence
  Description of Artificial Sequence Synthetic polypeptide
KCNTATCVLGKLSQELHRLQTYPRTNTGSNTY

SEQ ID NO: 60 - protein, 32 aa, Artificial Sequence
  Description of Artificial Sequence Synthetic polypeptide
KCNTAACVLGRLSQELHRLQTYPRTNTGSNTY

SEQ ID NO: 61 - protein, 32 aa, Artificial Sequence
  Description of Artificial Sequence Synthetic polypeptide
KCNTATCVLGRLSQELHKLQTYPRTNTGSNTY

SEQ ID NO: 62 - protein, 32 aa, Artificial Sequence
  Description of Artificial Sequence Synthetic polypeptide
KCNTATCVLGRLSQELHRLQTYPRTNTGSGTP

SEQ ID NO: 63 - protein, 32 aa, Artificial Sequence
  Description of Artificial Sequence Synthetic polypeptide
CSALSTCVLGRLSQELHRLQTYPRTNTGSNTY

SEQ ID NO: 64 - protein, 32 aa, Artificial Sequence; identical to SEQ ID NO: 6
  Description of Artificial Sequence Synthetic polypeptide

SEQ ID NO: 65 - protein, 32 aa, Artificial Sequence; identical to SEQ ID NO: 6
  Description of Artificial Sequence Synthetic polypeptide

SEQ ID NO: 66 - protein, 32 aa, Artificial Sequence
  Description of Artificial Sequence Synthetic polypeptide
KCNTATCVLGXLSQELHRLQTYPRTNTGSNTY

SEQ ID NO: 67 - protein, 33 aa, Artificial Sequence
  Description of Artificial Sequence Synthetic polypeptide
KCNTATCVLGRLSQELHRLQTYPRTNTGSNTYX

SEQ ID NO: 68 - protein, 32 aa, Artificial Sequence
  Description of Artificial Sequence Synthetic polypeptide
ACDTATCVLGRLSQELHRLQTYPRTNTGSNTY

SEQ ID NO: 69 - protein, 32 aa, Artificial Sequence
  Description of Artificial Sequence Synthetic polypeptide
KCNTATCVLGRLADALHRLQTYPRTNTGSNTY

SEQ ID NO: 70 - protein, 32 aa, Artificial Sequence
  Description of Artificial Sequence Synthetic polypeptide
KCNTATCVLGRLAQFLHRLQTYPRTNTGSNTY

SEQ ID NO: 71 - protein, 31 aa, Artificial Sequence
  Description of Artificial Sequence Synthetic polypeptide
CNTATCVLGRLADFLHRLQTYPRTNTGSNTY

SEQ ID NO: 72 - protein, 32 aa, Artificial Sequence
  Description of Artificial Sequence Synthetic polypeptide
SCNTATCVLGRLADALHRLQTMPRTNTGSNTY

SEQ ID NO: 73 - protein, 32 aa, Artificial Sequence
  Description of Artificial Sequence Synthetic polypeptide
KCNTATCVLGRLTDTLHRLQTYPRTNTGSNTY

SEQ ID NO: 74 - protein, 32 aa, Artificial Sequence
  Description of Artificial Sequence Synthetic polypeptide
KCNTATCVLGRLEEELHRLQTYPRTNTGSNTY

SEQ ID NO: 75 - protein, 32 aa, Artificial Sequence
  Description of Artificial Sequence Synthetic polypeptide
HCNTATCVLGRLEEELHRLQTYPRTNTGSNTY

SEQ ID NO: 76 - protein, 32 aa, Artificial Sequence
  Description of Artificial Sequence Synthetic polypeptide
FCNTATCVLGRLADFLHRLQTYPRTNTGSNTY

SEQ ID NO: 77 - protein, 33 aa, Artificial Sequence
  Description of Artificial Sequence Synthetic polypeptide
HGECNTATCVLGRLSQELHRLQTYPRTNTGSNT

SEQ ID NO: 78 - protein, 32 aa, Artificial Sequence
  Description of Artificial Sequence Synthetic polypeptide
KCNTATCLLQRLQKELQRLKQYPRTNTGSNTY

SEQ ID NO: 79 - protein, 33 aa, Artificial Sequence
  Description of Artificial Sequence Synthetic polypeptide
HEGCNTATCVLGRLSQELHRLQTYPRTNTGSNT

SEQ ID NO: 80 - protein, 32 aa, Artificial Sequence
  Description of Artificial Sequence Synthetic polypeptide
CSNLSTCATQRLANELVRLQTYPRTNVGSNTY

SEQ ID NO: 81 - protein, 32 aa, Artificial Sequence
  Description of Artificial Sequence Synthetic polypeptide
KCNTASCVLGRLSQELHRLQTYPRTNTGSNTY

SEQ ID NO: 82 - protein, 32 aa, Artificial Sequence
  Description of Artificial Sequence Synthetic polypeptide
KCNTAVCVLGRLSQELHRLQTYPRTNTGSNTY

SEQ ID NO: 83 - protein, 29 aa, Artificial Sequence
  Description of Artificial Sequence Synthetic peptide
KCNTATCVLGRLSQELHRYPRTNTGSNTY

SEQ ID NO: 84 - protein, 27 aa, Artificial Sequence
  Description of Artificial Sequence Synthetic peptide
KCNTATCVLGRLSQELYPRTNTGSNTY

SEQ ID NO: 85 - protein, 35 aa, Artificial Sequence
  Description of Artificial Sequence Synthetic polypeptide
KCNTATCVLGRLSQELHRLQTLQTYPRTNTGSNTY

SEQ ID NO: 86 - protein, 32 aa, Artificial Sequence
  Description of Artificial Sequence Synthetic polypeptide
KCNTATCVLGKLSQELHKLQTYPRTNTGSNTY

SEQ ID NO: 87 - protein, 32 aa, Artificial Sequence
  Description of Artificial Sequence Synthetic polypeptide
KCNTAXCVLGRLSQELHRLQTYPRTNTGSNTY

SEQ ID NO: 88 - protein, 32 aa, Artificial Sequence; identical to SEQ ID NO: 87
  Description of Artificial Sequence Synthetic polypeptide

SEQ ID NO: 89 - protein, 28 aa, Artificial Sequence
  Description of Artificial Sequence Synthetic peptide
STAVLGRLSQELHRLQTYPRTNTGSNTY

SEQ ID NO: 90 - protein, 32 aa, Artificial Sequence
  Description of Artificial Sequence Synthetic polypeptide
KCNTATCVLGXLSQELHXLQTYPRTNTGSNTY

SEQ ID NO: 91 - protein, 32 aa, Artificial Sequence; identical to SEQ ID NO: 90
  Description of Artificial Sequence Synthetic polypeptide

SEQ ID NO: 92 - protein, 34 aa, Artificial Sequence
  Description of Artificial Sequence Synthetic polypeptide
GCNTATCQVQNLSHRLWQLRQDSAPVEPSSPHSY

SEQ ID NO: 93 - protein, 31 aa, Artificial Sequence
  Description of Artificial Sequence Synthetic polypeptide
CNTATCVLGRLSQELHRLQTYPRTNTGSNTY

SEQ ID NO: 94 - protein, 32 aa, Artificial Sequence; identical to SEQ ID NO: 6
  Description of Artificial Sequence Synthetic polypeptide

SEQ ID NO: 95 - protein, 35 aa, Artificial Sequence
  Description of Artificial Sequence Synthetic polypeptide
FDAACNTATCVLGRLSQELHRLQTYPRTNTGSNTY

SEQ ID NO: 96 - protein, 32 aa, Artificial Sequence; identical to SEQ ID NO: 5
  Description of Artificial Sequence Synthetic polypeptide

SEQ ID NO: 97 - protein, 32 aa, Artificial Sequence
  Description of Artificial Sequence Synthetic polypeptide
SSNLSTSATQRLANELVRLQTYPRTNVGSNTY

SEQ ID NO: 98 - protein, 29 aa, Artificial Sequence
  Description of Artificial Sequence Synthetic peptide
LSTCVLGRLSQELHRLQTYPRTNTGSNTY

SEQ ID NO: 99 - protein, 29 aa, Artificial Sequence
  Description of Artificial Sequence Synthetic peptide
LSTSVLGRLSQELHRLQTYPRTNTGSNTY

SEQ ID NO: 100 - protein, 25 aa, Artificial Sequence
  Description of Artificial Sequence Synthetic peptide
VLGKLSQELNKFHTFPQTAIGVGAP

SEQ ID NO: 101 - protein, 30 aa, Artificial Sequence
  Description of Artificial Sequence Synthetic polypeptide
ATQRLANFLVRSSNNLGPVLPPTNVGSNTY

SEQ ID NO: 102 - protein, 29 aa, Artificial Sequence
  Description of Artificial Sequence Synthetic peptide
LSTSVLGRLSQELHRLQTYPRTNTGSNTY

SEQ ID NO: 103 - protein, 29 aa, Artificial Sequence
  Description of Artificial Sequence Synthetic peptide
LSTAVLGRLSQELHRLQTYPRTNTGSNTY

SEQ ID NO: 104 - protein, 24 aa, Artificial Sequence
  Description of Artificial Sequence Synthetic peptide
KCNTATCATQRLANFLVHSSNNGY

SEQ ID NO: 105 - protein, 32 aa, Artificial Sequence
  Description of Artificial Sequence Synthetic polypeptide
KCNTATCALQRLAQELHRLQALPRTNVGSNTY

SEQ ID NO: 106 - protein, 32 aa, Artificial Sequence
  Description of Artificial Sequence Synthetic polypeptide
KCNTATCALQRLSQELHRLQALPRTNVGSNTY

SEQ ID NO: 107 - protein, 32 aa, Artificial Sequence
  Description of Artificial Sequence Synthetic polypeptide
KCNTATCVLGRLAQELHRLQALPRTNVGSNTY

SEQ ID NO: 108 - protein, 33 aa, Artificial Sequence
  Description of Artificial Sequence Synthetic polypeptide
GKCNTATCVLGRLSQELHRLQTYPRTNTGSNTY

SEQ ID NO: 109 - protein, 32 aa, Artificial Sequence
  Description of Artificial Sequence Synthetic polypeptide
KCNTATCVLGRLSQELHRLQALPRTNVGSNTY

SEQ ID NO: 110 - protein, 32 aa, Artificial Sequence
  Description of Artificial Sequence Synthetic polypeptide
KCNTATCVLGRLAQELHRLQTYPRTNVGSNTY

SEQ ID NO: 111 - protein, 32 aa, Artificial Sequence
  Description of Artificial Sequence Synthetic polypeptide
KCNTATCALQRLSQELHRLQTYPRTNVGSNTY

SEQ ID NO: 112 - protein, 33 aa, Artificial Sequence
  Description of Artificial Sequence Synthetic polypeptide
KXCNTATCVLGRLSQELHRLQTYPRTNVGSNTY

SEQ ID NO: 113 - protein, 33 aa, Artificial Sequence; identical to SEQ ID NO: 112
  Description of Artificial Sequence Synthetic polypeptide

SEQ ID NO: 114 - protein, 33 aa, Artificial Sequence
  Description of Artificial Sequence Synthetic polypeptide
KCNTATCVLGRLSQELHKXLQTYPRTNVGSNTY

SEQ ID NO: 115 - protein, 33 aa, Artificial Sequence; identical to SEQ ID NO: 114
  Description of Artificial Sequence Synthetic polypeptide

SEQ ID NO: 116 - protein, 32 aa, Artificial Sequence
  Description of Artificial Sequence Synthetic polypeptide
KCNTATCXLQRLSQELHRLQTYPRTNVGSNTY

SEQ ID NO: 117 - protein, 32 aa, Artificial Sequence
  Description of Artificial Sequence Synthetic polypeptide
KCNTATCVLGRLXQELHRLQTYPRTNVGSNTY

SEQ ID NO: 118 - protein, 32 aa, Artificial Sequence
  Description of Artificial Sequence Synthetic polypeptide
KCNTATCXLQRLXQELHRLQTYPRTNVGSNTY

SEQ ID NO: 119 - protein, 32 aa, Artificial Sequence
  Description of Artificial Sequence Synthetic polypeptide
KCNTATCVLERLKQELHRLQTYPRTNVGSNTY

SEQ ID NO: 120 - protein, 32 aa, Artificial Sequence
  Description of Artificial Sequence Synthetic polypeptide
KCNTATCVLGRLSQELHKLQTYPRTNVGSNTY

SEQ ID NO: 121 - protein, 32 aa, Artificial Sequence
  Description of Artificial Sequence Synthetic polypeptide
KCNTATCVLGKLSQELHRLQTYPRTNVGSNTY

SEQ ID NO: 122 - protein, 32 aa, Artificial Sequence
  Description of Artificial Sequence Synthetic polypeptide
KCNTATCVLGRLSQELERLQKYPRTNVGSNTY

SEQ ID NO: 123 - protein, 32 aa, Artificial Sequence; identical to SEQ ID NO: 119
  Description of Artificial Sequence Synthetic polypeptide

SEQ ID NO: 124 - protein, 32 aa, Artificial Sequence
  Description of Artificial Sequence Synthetic polypeptide
KCNTATCVLGRLSQELERLKTYPRTNVGSNTY

SEQ ID NO: 125 - protein, 32 aa, Artificial Sequence
  Description of Artificial Sequence Synthetic polypeptide
KCNTATCVLERLSKELHRLQTYPRTNVGSNTY

SEQ ID NO: 126 - protein, 32 aa, Artificial Sequence; identical to SEQ ID NO: 121
  Description of Artificial Sequence Synthetic polypeptide

SEQ ID NO: 127 - protein, 32 aa, Artificial Sequence; identical to SEQ ID NO: 122
  Description of Artificial Sequence Synthetic polypeptide

SEQ ID NO: 128 - protein, 32 aa, Artificial Sequence; identical to SEQ ID NO: 125
  Description of Artificial Sequence Synthetic polypeptide

SEQ ID NO: 129 - protein, 38 aa, Artificial Sequence
  Description of Artificial Sequence Synthetic polypeptide
GAPPPSKCNTATCVLGRLSQELHRLQTYPRTNVGSNTY

SEQ ID NO: 130 - protein, 32 aa, Artificial Sequence; identical to SEQ ID NO: 124
  Description of Artificial Sequence Synthetic polypeptide

SEQ ID NO: 131 - protein, 32 aa, Artificial Sequence; identical to SEQ ID NO: 120
  Description of Artificial Sequence Synthetic polypeptide

SEQ ID NO: 132 - protein, 32 aa, Artificial Sequence
  Description of Artificial Sequence Synthetic polypeptide
KCNTATCALQRLADFLHRLQTYPRTNVGSNTY

SEQ ID NO: 133 - protein, 33 aa, Artificial Sequence
  Description of Artificial Sequence Synthetic polypeptide
GKCNTATCALQRLAQELHRLQTYPRTNVGSNTY

SEQ ID NO: 134 - protein, 33 aa, Artificial Sequence
  Description of Artificial Sequence Synthetic polypeptide
KCNTATCALQRLAQELHKXLQTYPRTNVGSNTY

SEQ ID NO: 135 - protein, 32 aa, Artificial Sequence
  Description of Artificial Sequence Synthetic polypeptide
KCNTATCVLGRLSQELHRLQHYPRTNVGSNTY

SEQ ID NO: 136 - protein, 32 aa, Artificial Sequence
  Description of Artificial Sequence Synthetic polypeptide
KCNTATCVLGRLHQELHRLQTYPRTNVGSNTY

SEQ ID NO: 137 - protein, 32 aa, Artificial Sequence
  Description of Artificial Sequence Synthetic polypeptide
KCNTATCVLGRLSQELHRLQTYARTNVGSNTY

SEQ ID NO: 138 - protein, 32 aa, Artificial Sequence
  Description of Artificial Sequence Synthetic polypeptide
KCNTATCVLGRLSQELHRLQTYPATNVGSNTY

SEQ ID NO: 139 - protein, 32 aa, Artificial Sequence
  Description of Artificial Sequence Synthetic polypeptide
KCNTATCVLGRLSQELHRLQTYPRANVGSNTY

SEQ ID NO: 140 - protein, 32 aa, Artificial Sequence
  Description of Artificial Sequence Synthetic polypeptide
KCNTATCVLGRLSQELHRLQTYPRTAVGSNTY

SEQ ID NO: 141 - protein, 32 aa, Artificial Sequence
  Description of Artificial Sequence Synthetic polypeptide
KCNTATCVLGRLSQELHRLQTYPRTNAGSNTY

SEQ ID NO: 142 - protein, 32 aa, Artificial Sequence
  Description of Artificial Sequence Synthetic polypeptide
KCNTATCVLGRLSQELHRLQTYPRTNVASNTY

SEQ ID NO: 143 - protein, 32 aa, Artificial Sequence
  Description of Artificial Sequence Synthetic polypeptide
KCNTATCVLGRLSQELHRLQTYPRTNVGANTY

SEQ ID NO: 144 - protein, 32 aa, Artificial Sequence
  Description of Artificial Sequence Synthetic polypeptide
KCNTATCVLGRLSQELHRLQTYPRTNVGSATY

SEQ ID NO: 145 - protein, 32 aa, Artificial Sequence
  Description of Artificial Sequence Synthetic polypeptide
KCNTATCVLGRLSQELHRLQTYPRTNVGSNAY

SEQ ID NO: 146 - protein, 32 aa, Artificial Sequence
  Description of Artificial Sequence Synthetic polypeptide
KCNTATCVLGRLSQELHRLQTYPRTNVGSNTA

SEQ ID NO: 147 - protein, 32 aa, Artificial Sequence
  Description of Artificial Sequence Synthetic polypeptide
KCNTATCVLGRLSQELHALQTYPRTNVGSNTY

SEQ ID NO: 148 - protein, 32 aa, Artificial Sequence
  Description of Artificial Sequence Synthetic polypeptide
KCNTATCVLGRLSQELHRAQTYPRTNVGSNTY

SEQ ID NO: 149 - protein, 32 aa, Artificial Sequence
  Description of Artificial Sequence Synthetic polypeptide
KCNTATCVLGRLSQELHRLATYPRTNVGSNTY

SEQ ID NO: 150 - protein, 32 aa, Artificial Sequence
  Description of Artificial Sequence Synthetic polypeptide
KCNTATCVLGRLSQELHRLQAYPRTNVGSNTY

SEQ ID NO: 151 - protein, 32 aa, Artificial Sequence
  Description of Artificial Sequence Synthetic polypeptide
KCNTATCVLGRLSQELHRLQTAPRTNVGSNTY

SEQ ID NO: 152 - protein, 32 aa, Artificial Sequence
  Description of Artificial Sequence Synthetic polypeptide
KCNTATCVLGRLSQALHRLQTYPRTNVGSNTY

SEQ ID NO: 153 - protein, 32 aa, Artificial Sequence
  Description of Artificial Sequence Synthetic polypeptide
KCNTATCVLGRLSQEAHRLQTYPRTNVGSNTY

SEQ ID NO: 154 - protein, 32 aa, Artificial Sequence
  Description of Artificial Sequence Synthetic polypeptide
KCNTATCVLGRLSQELARLQTYPRTNVGSNTY

SEQ ID NO: 155 - protein, 32 aa, Artificial Sequence
  Description of Artificial Sequence Synthetic polypeptide
SCNTATCVLGRLADALHRLQTLPRTNTGSNTY

SEQ ID NO: 156 - protein, 32 aa, Artificial Sequence
  Description of Artificial Sequence Synthetic polypeptide
SCNTATCVLGRLAEALHRLQTLPRTNTGSNTY

SEQ ID NO: 157 - protein, 32 aa, Artificial Sequence
  Description of Artificial Sequence Synthetic polypeptide
SCNTATCVLGRLEEALHRLQTLPRTNTGSNTY

SEQ ID NO: 158 - protein, 32 aa, Artificial Sequence
  Description of Artificial Sequence Synthetic polypeptide
SCNTATCALQRLADALHRLQTLPRTNTGSNTY

SEQ ID NO: 159 - protein, 32 aa, Artificial Sequence
  Description of Artificial Sequence Synthetic polypeptide
SCNTATCALQRLAEALHRLQTLPRTNTGSNTY

SEQ ID NO: 160 - protein, 35 aa, Artificial Sequence
  Description of Artificial Sequence Synthetic polypeptide
KCNTATCVLGRLSQELHRAQTLQTYPRTNTGSNTY

SEQ ID NO: 161 - protein, 35 aa, Artificial Sequence
  Description of Artificial Sequence Synthetic polypeptide
KCNTATCVLGRLSQELHRLQTLQTYPRTNVGSNTY

SEQ ID NO: 162 - protein, 35 aa, Artificial Sequence
  Description of Artificial Sequence Synthetic polypeptide
KCNTATCVLGRLSQELHRAQTLQTYPRTNVGSNTY

SEQ ID NO: 163 - protein, 35 aa, Artificial Sequence
  Description of Artificial Sequence Synthetic polypeptide
KCNTATCVLGRLADALHRLQTLQTYPRTNTGSNTY

SEQ ID NO: 164 - protein, 32 aa, Artificial Sequence
  Description of Artificial Sequence Synthetic polypeptide
KCNTATCIDLTFHLLRTLLELAPRTNTGSNTY

SEQ ID NO: 165 - protein, 37 aa, Artificial Sequence
  Description of Artificial Sequence Synthetic polypeptide
KCNTATCIDLTFHLLRTLLELARTQSQPRTNTGSNTY

SEQ ID NO: 166 - protein, 32 aa, Artificial Sequence
  Description of Artificial Sequence Synthetic polypeptide
DNPSLSVLGRLSQELHRLQTYAEQNRIIFDSV

SEQ ID NO: 167 - protein, 41 aa, Artificial Sequence
  Description of Artificial Sequence Synthetic polypeptide
KCNTATCVLGRLSQELHRLQTYRTQSQRERAEQNRIIFDSV

SEQ ID NO: 168 - protein, 31 aa, Artificial Sequence
  Description of Artificial Sequence Synthetic polypeptide
DNPSLSIDLTFHLLRTLLELAPRTNTGSNTY

SEQ ID NO: 169 - protein, 32 aa, Artificial Sequence
  Description of Artificial Sequence Synthetic polypeptide
SCNTATCVLGRLAEALHRLQKLPRTNTGSNTY

SEQ ID NO: 170 - protein, 31 aa, Artificial Sequence; identical to SEQ ID NO: 21
  Description of Artificial Sequence Synthetic polypeptide

SEQ ID NO: 171 - protein, 31 aa, Artificial Sequence; identical to SEQ ID NO: 21
  Description of Artificial Sequence Synthetic polypeptide

SEQ ID NO: 172 - protein, 31 aa, Artificial Sequence; identical to SEQ ID NO: 21
  Description of Artificial Sequence Synthetic polypeptide

SEQ ID NO: 173 - protein, 31 aa, Artificial Sequence; identical to SEQ ID NO: 21
  Description of Artificial Sequence Synthetic polypeptide

SEQ ID NO: 174 - protein, 32 aa, Artificial Sequence; identical to SEQ ID NO: 20
  Description of Artificial Sequence Synthetic polypeptide

SEQ ID NO: 175 - protein, 31 aa, Artificial Sequence; identical to SEQ ID NO: 31
  Description of Artificial Sequence Synthetic polypeptide

SEQ ID NO: 176 - protein, 34 aa, Artificial Sequence; identical to SEQ ID NO: 29
  Description of Artificial Sequence Synthetic polypeptide

SEQ ID NO: 177 - protein, 32 aa, Artificial Sequence; identical to SEQ ID NO: 35
  Description of Artificial Sequence Synthetic polypeptide

SEQ ID NO: 178 - protein, 31 aa, Artificial Sequence; identical to SEQ ID NO: 33
  Description of Artificial Sequence Synthetic polypeptide

SEQ ID NO: 179 - protein, 31 aa, Artificial Sequence; identical to SEQ ID NO: 21
  Description of Artificial Sequence Synthetic polypeptide

SEQ ID NO: 180 - protein, 31 aa, Artificial Sequence; identical to SEQ ID NO: 21
  Description of Artificial Sequence Synthetic polypeptide

SEQ ID NO: 181 - protein, 31 aa, Artificial Sequence; identical to SEQ ID NO: 21
  Description of Artificial Sequence Synthetic polypeptide

SEQ ID NO: 182 - protein, 31 aa, Artificial Sequence; identical to SEQ ID NO: 21
  Description of Artificial Sequence Synthetic polypeptide

SEQ ID NO: 183 - protein, 31 aa, Artificial Sequence; identical to SEQ ID NO: 21
  Description of Artificial Sequence Synthetic polypeptide

SEQ ID NO: 184 - protein, 31 aa, Artificial Sequence; identical to SEQ ID NO: 21
  Description of Artificial Sequence Synthetic polypeptide

SEQ ID NO: 185 - protein, 31 aa, Artificial Sequence; identical to SEQ ID NO: 21
  Description of Artificial Sequence Synthetic polypeptide

SEQ ID NO: 186 - protein, 31 aa, Artificial Sequence; identical to SEQ ID NO: 21
  Description of Artificial Sequence Synthetic polypeptide

SEQ ID NO: 187 - protein, 31 aa, Artificial Sequence; identical to SEQ ID NO: 21
  Description of Artificial Sequence Synthetic polypeptide

SEQ ID NO: 188 - protein, 31 aa, Artificial Sequence; identical to SEQ ID NO: 34
  Description of Artificial Sequence Synthetic polypeptide

SEQ ID NO: 189 - protein, 31 aa, Artificial Sequence; identical to SEQ ID NO: 25
  Description of Artificial Sequence Synthetic polypeptide

SEQ ID NO: 190 - protein, 31 aa, Artificial Sequence; identical to SEQ ID NO: 26
  Description of Artificial Sequence Synthetic polypeptide

SEQ ID NO: 191 - protein, 31 aa, Artificial Sequence; identical to SEQ ID NO: 23
  Description of Artificial Sequence Synthetic polypeptide

SEQ ID NO: 192 - protein, 31 aa, Artificial Sequence; identical to SEQ ID NO: 24
  Description of Artificial Sequence Synthetic polypeptide

SEQ ID NO: 193 - protein, 31 aa, Artificial Sequence; identical to SEQ ID NO: 27
  Description of Artificial Sequence Synthetic polypeptide

SEQ ID NO: 194 - protein, 31 aa, Artificial Sequence; identical to SEQ ID NO: 31
  Description of Artificial Sequence Synthetic polypeptide

SEQ ID NO: 195 - protein, 31 aa, Artificial Sequence; identical to SEQ ID NO: 31
  Description of Artificial Sequence Synthetic polypeptide

SEQ ID NO: 196 - protein, 35 aa, Artificial Sequence
  Description of Artificial Sequence Synthetic polypeptide
CNTATCVLGRLSQELHRLQKGGGTYPRTNVGSNTY

SEQ ID NO: 197 - protein, 31 aa, Artificial Sequence; identical to SEQ ID NO: 31
  Description of Artificial Sequence Synthetic polypeptide

SEQ ID NO: 198 - protein, 31 aa, Artificial Sequence; identical to SEQ ID NO: 34
  Description of Artificial Sequence Synthetic polypeptide

SEQ ID NO: 199 - protein, 31 aa, Artificial Sequence; identical to SEQ ID NO: 34
  Description of Artificial Sequence Synthetic polypeptide

SEQ ID NO: 200 - protein, 31 aa, Artificial Sequence; identical to SEQ ID NO: 34
  Description of Artificial Sequence Synthetic polypeptide

SEQ ID NO: 201 - protein, 31 aa, Artificial Sequence; identical to SEQ ID NO: 34
  Description of Artificial Sequence Synthetic polypeptide

SEQ ID NO: 202 - protein, 31 aa, Artificial Sequence; identical to SEQ ID NO: 34
  Description of Artificial Sequence Synthetic polypeptide

SEQ ID NO: 203 - protein, 32 aa, Artificial Sequence; identical to SEQ ID NO: 120
  Description of Artificial Sequence Synthetic polypeptide

SEQ ID NO: 204 - protein, 32 aa, Artificial Sequence
  Description of Artificial Sequence Synthetic polypeptide
KCNTATCVLGRLSQELHKLQKYPRTNTGSNTY

SEQ ID NO: 205 - protein, 32 aa, Artificial Sequence
  Description of Artificial Sequence Synthetic polypeptide
KCNTATCALQRLAQELHKLQTYPRTNVGSNTY

SEQ ID NO: 206 - protein, 32 aa, Artificial Sequence
  Description of Artificial Sequence Synthetic polypeptide
KCNTATCALQRLAQELHRLQKYPRTNVGSNTY

SEQ ID NO: 207 - protein, 32 aa, Artificial Sequence
  Description of Artificial Sequence Synthetic polypeptide
KCNTATCALQRLAQELHRLQTYPKTNVGSNTY

SEQ ID NO: 208 - protein, 32 aa, Artificial Sequence
  Description of Artificial Sequence Synthetic polypeptide
KCNTATCALQRLAQELHRLQTYPRTKVGSNTY

SEQ ID NO: 209 - protein, 32 aa, Artificial Sequence; identical to SEQ ID NO: 205
  Description of Artificial Sequence Synthetic polypeptide

SEQ ID NO: 210 - protein, 32 aa, Artificial Sequence; identical to SEQ ID NO: 205
  Description of Artificial Sequence Synthetic polypeptide

SEQ ID NO: 211 - protein, 32 aa, Artificial Sequence; identical to SEQ ID NO: 36
  Description of Artificial Sequence Synthetic polypeptide

SEQ ID NO: 212 - protein, 32 aa, Artificial Sequence
  Description of Artificial Sequence Synthetic polypeptide
KCNTATCALQKLAQELHRLQTYPRTNVGSNTY

SEQ ID NO: 213 - protein, 32 aa, Artificial Sequence; identical to SEQ ID NO: 205
  Description of Artificial Sequence Synthetic polypeptide

SEQ ID NO: 214 - protein, 33 aa, Artificial Sequence
  Description of Artificial Sequence Synthetic polypeptide
KECNTATCALQRLAQELHRLQTYPRTNVGSNTY